SEQUENCE LISTING (45 sequences)

SEQ ID NO: 1 - DNA, 20 bp, Artificial Sequence
  Description of Artificial Sequence Synthetic primer
gtcccggattttactggttc

SEQ ID NO: 2 - DNA, 20 bp, Artificial Sequence
  Description of Artificial Sequence Synthetic primer
gtacctccaggaagccatca

SEQ ID NO: 3 - DNA, 20 bp, Artificial Sequence
  Description of Artificial Sequence Synthetic primer
ggtgggaaacgtcctagtca

SEQ ID NO: 4 - DNA, 20 bp, Artificial Sequence
  Description of Artificial Sequence Synthetic primer
gaaaacggctgcatattggt

SEQ ID NO: 5 - DNA, 20 bp, Artificial Sequence
  Description of Artificial Sequence Synthetic primer
atcaggtggtgaaaggcaag

SEQ ID NO: 6 - DNA, 20 bp, Artificial Sequence
  Description of Artificial Sequence Synthetic primer
gagactgcgaaacgatcctc

SEQ ID NO: 7 - DNA, 21 bp, Artificial Sequence
  Description of Artificial Sequence Synthetic primer
aaccacagagcaagatcagga

SEQ ID NO: 8 - DNA, 20 bp, Artificial Sequence
  Description of Artificial Sequence Synthetic primer
gcatgttccagaccatcaaa

SEQ ID NO: 9 - DNA, 20 bp, Artificial Sequence
  Description of Artificial Sequence Synthetic primer
aaaggaggagctggctaagg

SEQ ID NO: 10 - DNA, 20 bp, Artificial Sequence
  Description of Artificial Sequence Synthetic primer
agtccacggtctggtcttca

SEQ ID NO: 11 - DNA, 20 bp, Artificial Sequence
  Description of Artificial Sequence Synthetic primer
tgacccttgccttggaaata

SEQ ID NO: 12 - DNA, 20 bp, Artificial Sequence
  Description of Artificial Sequence Synthetic primer
ccagctttggaagtgtcctc

SEQ ID NO: 13 - DNA, 20 bp, Artificial Sequence
  Description of Artificial Sequence Synthetic primer
ccactgtctgtgtccgtgtc

SEQ ID NO: 14 - DNA, 21 bp, Artificial Sequence
  Description of Artificial Sequence Synthetic primer
tgcctcaagattttctggttt

SEQ ID NO: 15 - DNA, 20 bp, Artificial Sequence
  Description of Artificial Sequence Synthetic primer
gcaagttccgtccagtcatt

SEQ ID NO: 16 - DNA, 20 bp, Artificial Sequence
  Description of Artificial Sequence Synthetic primer
gtctgtctgcgtgtgctgtt

SEQ ID NO: 17 - DNA, 20 bp, Artificial Sequence
  Description of Artificial Sequence Synthetic primer
cattccaagcagagcaaaca

SEQ ID NO: 18 - DNA, 20 bp, Artificial Sequence
  Description of Artificial Sequence Synthetic primer
gcaagctgcatagccttctc

SEQ ID NO: 19 - DNA, 19 bp, Artificial Sequence
  Description of Artificial Sequence Synthetic primer
gctgcaggaacatctggac

SEQ ID NO: 20 - DNA, 20 bp, Artificial Sequence
  Description of Artificial Sequence Synthetic primer
accagaacctgaccacagga

SEQ ID NO: 21 - DNA, 20 bp, Artificial Sequence
  Description of Artificial Sequence Synthetic primer
aggctccatcaccaacaatc

SEQ ID NO: 22 - DNA, 20 bp, Artificial Sequence
  Description of Artificial Sequence Synthetic primer
catcacagaggacacctcca

SEQ ID NO: 23 - DNA, 20 bp, Artificial Sequence
  Description of Artificial Sequence Synthetic primer
tgggaactcagaccgtacct

SEQ ID NO: 24 - DNA, 20 bp, Artificial Sequence
  Description of Artificial Sequence Synthetic primer
atcaccgacagcacagacag

SEQ ID NO: 25 - DNA, 20 bp, Artificial Sequence
  Description of Artificial Sequence Synthetic primer
ctcaaggaacctccaccagg

SEQ ID NO: 26 - DNA, 20 bp, Artificial Sequence
  Description of Artificial Sequence Synthetic primer
gtatcctggctggtgtcctc

SEQ ID NO: 27 - DNA, 20 bp, Artificial Sequence
  Description of Artificial Sequence Synthetic primer
gatttcggagtggtgaagga

SEQ ID NO: 28 - DNA, 20 bp, Artificial Sequence
  Description of Artificial Sequence Synthetic primer
aagagggcattttggttgtg

SEQ ID NO: 29 - DNA, 20 bp, Artificial Sequence
  Description of Artificial Sequence Synthetic primer
ccatcgacagccctgatagt

SEQ ID NO: 30 - DNA, 20 bp, Artificial Sequence
  Description of Artificial Sequence Synthetic primer
cccatcctcacttcctcaac

SEQ ID NO: 31 - DNA, 20 bp, Artificial Sequence
  Description of Artificial Sequence Synthetic primer
gggtaacagatccccgtttt

SEQ ID NO: 32 - DNA, 20 bp, Artificial Sequence
  Description of Artificial Sequence Synthetic primer
ttgacggtgtgatggaagtg

SEQ ID NO: 33 - DNA, 19 bp, Artificial Sequence
  Description of Artificial Sequence Synthetic primer
gcccgacaaatgggctggg

SEQ ID NO: 34 - DNA, 20 bp, Artificial Sequence
  Description of Artificial Sequence Synthetic primer
tagcctagcagcgtgtcctc

SEQ ID NO: 35 - DNA, 20 bp, Artificial Sequence
  Description of Artificial Sequence Synthetic primer
ggttgtgtttgctccacctt

SEQ ID NO: 36 - DNA, 20 bp, Artificial Sequence
  Description of Artificial Sequence Synthetic primer
gccaccaactatccagacca

SEQ ID NO: 37 - DNA, 20 bp, Artificial Sequence
  Description of Artificial Sequence Synthetic primer
tggcaaaggaggtaaaggtg

SEQ ID NO: 38 - DNA, 20 bp, Artificial Sequence
  Description of Artificial Sequence Synthetic primer
cagctccgatgggttatcag

SEQ ID NO: 39 - DNA, 20 bp, Artificial Sequence
  Description of Artificial Sequence Synthetic primer
ctcggtcagaaattgggaaa

SEQ ID NO: 40 - DNA, 20 bp, Artificial Sequence
  Description of Artificial Sequence Synthetic primer
gccttgtgtgctcatcattc

SEQ ID NO: 41 - DNA, 20 bp, Artificial Sequence
  Description of Artificial Sequence Synthetic primer
gctcagctacagtttcacag

SEQ ID NO: 42 - DNA, 20 bp, Artificial Sequence
  Description of Artificial Sequence Synthetic primer
caaataagcctccccttggt

SEQ ID NO: 43 - DNA, 20 bp, Artificial Sequence
  Description of Artificial Sequence Synthetic primer
agtaggtcatgggcctgttg

SEQ ID NO: 44 - DNA, 19 bp, Artificial Sequence
  Description of Artificial Sequence Synthetic primer
ccacgccagccatggttgt

SEQ ID NO: 45 - protein, 6 aa, Artificial Sequence
  Description of Artificial Sequence Synthetic 6xHis tag
HHHHHH

(19) United States
(12) Patent Application Publication
(10) Pub. No.: US 2015/0005191 A1
(43) Pub. Date: Jan. 1, 2015

(54) Compositions and Methods for Identifying Autism Spectrum Disorders

(71) Applicant: George Washington University, Washington, DC (US)

(72) Inventor: Valerie Wailin HU, Rockville, MD (US)

(21) Appl. No.: 14/477,442

(22) Filed: Sep. 4, 2014

Related U.S. Application Data
(63) Continuation of application No. 14/074,283, filed on Nov. 7, 2013.
Application No. 14/074,283 is a division of application No. 13/129,687, filed on Aug. 8, 2011, now abandoned.
(60) Provisional application No. 61/171,510, filed on Apr. 22, 2009.
(60) Provisional application No. 61/115,184, filed on Nov. 17, 2008.

(51) Int. Cl. C12Q 1/68; G06F 17/30

(52) U.S. Cl. CPC C12Q 1/6881; G06F 17/30289; C12Q 2600/112; C12Q 2600/158
USPC 506/9; 506/16; 707/803

(57) ABSTRACT

The compositions and methods described are directed to gene chips having a plurality of different oligonucleotides with specificity for genes associated with autism spectrum disorders. The invention further provides methods of identifying gene profiles for neurological and psychiatric conditions including autism spectrum disorders, methods of treating such conditions, and methods of identifying therapeutics for the treatment of such neurological and psychiatric conditions.

### CROSS REFERENCE TO RELATED APPLICATIONS

This application is a divisional of U.S. application Ser. No. 13/129,687, filed Aug. 8, 2011, which is a national stage of PCT Application No. PCT/US2009/064370, filed Nov. 13, 2009, which claims priority under 35 U.S.C. 119 to U.S. Provisional Application No. 61/115,184 filed Nov. 17, 2008, and U.S. Provisional Application No. 61/171,510 filed Apr. 22, 2009, the entire contents of which are incorporated herein by reference in their entirety.

### SEQUENCE LISTING

The instant application contains a Sequence Listing which has been submitted m ASCII format via EFS-Web and is hereby incorporated by reference in its entirety. Said ASCII copy, created on Jun. 13, 2011, is named 31778634.txt and is 10,514 bytes in size.

### FIELD OF THE INVENTION

This invention relates to DNA microarray technology, and more specifically to methods and kits for identifying autism and autism spectrum disorders in humans.

### BACKGROUND OF THE INVENTION

Autism spectrum disorders (ASD) are developmental disabilities resulting from dysfunction in the central nervous system and are characterized by impairments in three behavioral areas: communication (notably spoken language), social interactions, and repetitive behaviors or restricted interests (Volkmar F R, et al (1994)). ASD usually manifest before three years of age and the severity can vary greatly. Idiopathic ASD include autism, which is considered to be the most severe form, pervasive developmental disorders not otherwise specified (PDD-NOS), and Asperger's syndrome, a milder form of autism in which persons can have relatively normal intelligence and communication skills but difficulty with social interactions. ASD with defined genetic etiologies or chromosomal aberration include Rett's syndrome, tuberous sclerosis, Fragile X syndrome, and chromosome 15 duplication (reviewed in (Muhle R, Trentacoste S V & Rapin I (2004))). Familial studies provide evidence that individuals closely related to an autistic individual (i.e. mother, father, and siblings) may have “autistic tendencies” but do not meet criterion for ASD, suggesting that a broad autism phenotype (BAP) may also exist (Piven J, Palmer P, Jacobi D, Childress D & Arndt S (1997)).

Previous studies establish a strong genetic component for the etiology of autism, and many loci have been proposed as autism susceptibility regions, including loci on chromosomes 1, 2, 7, 11, 13, 15, 16, 17 (reviewed in (Polleux F & Lauder J M (2004), Yonan A L, et al (2003), Santangelo S L & Tsatsanis K (2005), and Gupta A R & State M W (2007)). However, the specific genes involved within each locus have not been determined to date. Available data further suggests that multiple gene interactions, epigenetic factors, and environmental risk factors may also be at the core of autism etiology (Lathe R (2006)).

Heterogeneity in phenotypic presentation of ASD has been used as one explanation for the difficulty in pinpointing chromosomal loci and genes involved in autism. Thus, recent studies have attempted to reduce the “noise” in genetic data by reducing the phenotypic heterogeneity of the sample population using a variety of approaches. Some of the earlier studies stratified samples for genetic analyses primarily on language deficits of the proband (eg., age at first word, phrase speech delay), while other studies focused on other attributes of autistic disorder, such as compulsions, or Restricted and Repetitive Stereotyped Behaviors (RRSB) to restrict phenotypic heterogeneity (Alarcon M, Cantor R M, Liu J, Gilliam T C & Geschwind D H (2002), Bradford Y, et al (2001), Silverman J M, et al (2001), Hollander E, et al (2000)). Another strategy for increasing the probability of observing genetic linkage was based upon the use of “endophenotypes” for specific autism-associated behaviors which were present in nonaffected family members (Spence S J, et al (2006)). Using this approach, Alarcon et al. and Chen et al. reported quantitative trait loci (QTL) for language and nonverbal communication deficits, respectively (Alarcon M, Yonan A L, Gilliam T C, Cantor R M & Geschwind D H (2005), Chen G K, Kono N, Geschwind D H & Cantor R M (2006)).

The Autism Diagnostic Interview-Revised (ADI-R) is a diagnostic screen for ASD which is a parent questionnaire that probes for language, social, behavioral, and functional abnormalities that are inconsistent with a specific child's stage of development (Lord C, Rutter M & Couteur A L (1994)). Principal components analysis (PCA) of 98 items from the Autism Diagnostic Interview-Revised (ADI-R) has also been used as a means to isolate genetically relevant phenotypes (Tadevosyan-Leyfer O, et al (2003)). This study identified 6 “factors” which accounted for 41% of the variation in the autistic population studied. Reexamination of genetic data from individuals defined by presence or absence “savant skills” (one of the factors) showed an increase in LOD score (0.4→2.6) in the chromosome 15q11-q13 region relative to the combined unsegregated sample population (Nurmi E L, et al (2003)). However, this finding could not be replicated by another group (Ma D Q, et al (2005)). A recent analysis of the use of the ADIR to increase phenotypic homogeneity summarizes the major studies which have attempted to stratify autism samples and further cautions that such stratification based upon a few defined attributes can also lead to unintended associations with other variables, such as age, gender, race, etc. (Lecavalier L, et al (2006)).

Thus, there is a need for systems and methods that will provide an increased understanding of the pathophysiology of Autism spectrum disorders, such as autism, pervasive developmental disorders not otherwise specified (PDD-NOS), and Asperger's syndrome, and their treatment.

The present invention demonstrates herein the use of multiple clustering methods applied to a broad range of ADIR items from a large population (1954 individuals) to identify subgroups of autistic individuals with clinically relevant behavioral phenotypes. Data from large-scale gene expression analyses on lymphoblastoid cell lines derived from individuals who fall within 3 of these subgroups show distinct differences in gene expression profiles that in part relate to the severity of the phenotype. Functional and pathway analyses of gene expression profiles associated with the phenotypic subgroups also suggest distinct differences in the biological phenotypes that associate with these subgroups. Based on these analyses, the present invention suggests that multivariate analysis of the ADIR data using a broad spectrum of the ADIR items and a combination of clustering methods that are typically employed in DNA microarray analyses may be an effective means of reducing the phenotypic heterogeneity of the sample population without restricting the phenotype to only one or a few items. Such an approach towards stratification of individuals which utilizes the full spectrum of autism-associated behaviors is expected to aid in the association of genetic and other biological phenotypes with specific forms of ASD.

Using these combined methods to identify both severe and mild subgroups of ASD individuals as well as those with notable savant skills, the present invention provides discrimination of autistic from nonautistic individuals based upon gene expression profiles. The present invention utilizes multivariate analysis to ultimately identify five transcripts that were significantly uniquely expressed in individuals with ASD. Finally, the present invention provides for comparison of gene expression profiles in cultured cells from autistic individuals and their respective non-autistic siblings to identify genes that may explain the biology underlying autism spectrum disorders

### SUMMARY OF THE INVENTION

One aspect of the invention provides a gene chip array having a plurality of different oligonucleotides with specificity for genes associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof.

In one embodiment of the present invention, a gene chip array is provided wherein the oligonucleotides are specific for the genes set out in Table 3, Table 7, Table 8, Table 9, Table 10, Table 18, Table 19, Table 21, Table 22, Table 23, Table 25, Table 26, Table 27, or Table 28, or a combination thereof.

In another aspect of the invention, a method is provided for screening a subject for a neurological disease or disorder comprising the steps of: (a) isolating a nucleic acid, protein or cellular extract from at least one cell from the subject; (b) measuring the gene expression level of at least five different genes in Table 3, Table 7, Table 8, Table 9, Table 10, Table 18, Table 19, Table 21, Table 22, Table 23, Table 25, Table 26, Table 27, or Table 28, or a combination thereof in the sample, wherein the at least five different genes have been determined to have differential expression in subjects with a neurological disease or disorder, wherein the subject is diagnosed to be at risk for or affected by a neurological disease or disorder if there is a statistically significant difference in the gene expression level in the at least five different genes in the sample compared to the gene expression level of the same genes from a healthy individual.

In one embodiment of the screening method of the present invention, the neurological disease comprises at least one autism spectrum disorder, autistic disorder, pervasive developmental disorder-not otherwise specified (PDD-NOS) including atypical autism, Asperger's Disorder, or a combination thereof.

In another embodiment of the screening method of the present invention, the at least 5 different genes in Table 3, Table 7, Table 8, Table 9, Table 10, Table 18, Table 19, Table 21, Table 22, Table 23, Table 25, Table 26, Table 27, or Table 28, or a combination thereof comprise genes involved in nervous system development, axon guidance, synaptic transmission or plasticity, myelination, long-term potentiation, neuron toxicity, embryonic development, regulation of actin networks, KEGG pathway, digestion, liver toxicity (hepatic stellate cell activation, fibrosis, and cholestasis), inflammation, oxidative stress, epilepsy, apoptosis, cell survival, differentiation, the unfolded protein response, Type II diabetes and insulin signaling, endocrine function, circadian rhythm, cholesterol metabolism and the steroidogenesis pathway, or a combination thereof.

In yet another embodiment of the screening method of the present invention, the healthy individual is a non-phenotypic discordant twin or sibling of the subject.

In yet another embodiment of the screening method of the present invention, the method distinguishes between different variants of autism spectrum disorder comprising a lower severity scores across all ADIR items, an intermediate severity across all ADIR items, a higher severity scores on spoken language items on the ADIR, a higher frequency of savant skills, and a severe language impairment, or a combination thereof.

In yet another embodiment of the screening method of the present invention, the gene expression is quantified with an assay comprising large scale microarray analysis, RT qPCR analysis, quantitative nuclease protection assay (qNPA) analysis, Western analysis, and focused gene chip analysis, in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift assay (EMSA), radioimmunoassay (RIA), fluorescent or histochemical staining, microscopy and digital image analysis, and fluorescence activated cell analysis or sorting (FACS), nucleic acid hybridization, antibody binding, or a combination thereof.

In yet another aspect of the invention, a method is provided for determining a gene profile for at least one autism spectrum disorder, comprising (a) preparing samples of control and experimental cDNA, wherein the experimental cDNA is generated from a nucleic acid sample isolated from a subject suspected of being afflicted with the at least one autism spectrum disorder and the control CDNA is generated from a nucleic acid sample isolated from a healthy individual; (b) preparing one or more microarrays comprising a plurality of different oligonucleotides having specificity for genes associated with the at least one autism spectrum disorder; (c) applying the prepared samples to the one or more microarrays to allow hybridization between the oligonucleotides and the control CDNA and the oligonucleotide and the experimental cDNAs; (d) identifying the oligonucleotides on the microarray which display differential hybridization to the experimental cDNA relative to the control cDNA thereby determining a gene profile for the at least one autism spectrum disorder.

In one embodiment of the gene profiling method of the present invention, the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

In another embodiment of the gene profiling method of the present invention, the at least one autism spectrum disorder comprises autistic disorder, pervasive developmental disorder-not otherwise specified (PDD-NOS), including atypical autism, Asperger's Disorder, or a combination thereof.

In yet another aspect of the invention, a method is provided for distinguishing between different phenotypes of an autism spectrum disorder comprising severely language impaired (L), mildly affected (M), or “savants” (S) comprising (a) preparing samples of control and experimental cDNA, wherein the experimental cDNA is generated from a nucleic acid sample isolated from a subject suspected of being afflicted with at least one phenotype comprising the severely language impaired (L), mildly affected (M), or “savants” (S); (b) preparing one or more microarrays comprising a plurality of different oligonucleotides having specificity for genes associated with the at least one phenotype; (c) applying the prepared samples to the one or more microarrays to allow hybridization between the oligonucleotides and the control and experimental cDNAs; (d) identifying the oligonucleotides on the microarray which display differential hybridization to the experimental cDNA relative to the control cDNA thereby determining a gene profile for distinguishing among the different phenotypes of autism spectrum disorder.

In another embodiment of the phenotype distinguishing method of the present invention, the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

In yet another embodiment of the phenotype distinguishing method of the present invention, the at least one autism spectrum disorder comprises autistic disorder, pervasive developmental disorder-not otherwise specified (PDD-NOS), including atypical autism, Asperger's Disorder, or a combination thereof.

In yet another aspect of the invention, a method is provided for predicting efficacy of a test compound for altering a behavioral response in a subject with at least one autism spectrum disorder comprising: (a) preparing a microarray comprising a plurality of different oligonucleotides, wherein the oligonucleotides are specific to genes associated with an autism spectrum disorder; (b) obtaining a gene profile representative of the gene expression profile of at least one sample of a selected tissue type from a subject subjected to each of at least one of a plurality of selected behavioral therapies which promote the behavioral response; (c) administering the test compound to the subject; and (d) comparing gene expression profile data in at least one sample of the selected tissue type from the subject treated with the test compound to determine a degree of similarity with one or more gene profiles associated with an autism spectrum disorder; wherein the predicted efficacy of the test compound for altering the behavioral response is correlated to said degree of similarity.

In another embodiment of the compound efficacy testing method of the present invention, the plurality of oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

In yet another embodiment of the compound efficacy testing method of the present invention, the autism spectrum disorder neurological condition comprises autistic disorder, pervasive developmental disorder-not otherwise specified (PDD-NOS), including atypical autism, Asperger's Disorder, or a combination thereof.

In yet another embodiment of the compound efficacy testing method of the present invention, step (a) comprises obtaining a gene profile representative of the gene expression profile of at least two samples of a selected tissue type.

In yet another embodiment of the compound efficacy testing method of the present invention, the selected tissue type comprises a neuronal tissue type.

In yet another embodiment of the compound efficacy testing method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

In yet another embodiment of the compound efficacy testing method of the present invention, the selected tissue type is selected from the group consisting of lymphocytes, blood, or mucosal epithelial cells, brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue.

In yet another embodiment of the compound efficacy testing method of the present invention, the test compound is an antibody, a nucleic acid molecule, a small molecule drug, or a nutritional or herbal supplement.

In yet another embodiment of the compound efficacy testing method of the present invention, the behavioral therapy comprises applied behavior analysis (ABA) intervention methods, dietary changes, exercise, massage therapy, group therapy, talk therapy, play therapy, conditioning, or alternative therapies such as sensory integration and auditory integration therapies.

In yet another aspect of the invention a method is provided for assessing the efficacy of a treatment in an individual having at least one autism spectrum disorder comprising (a) determining differential gene expression profile data specific for at least five difference genes set out in Table 3, Table 7, Table 8, Table 9, Table 10, Table 18, Table 19, Table 21, Table 22, Table 23, Table 25, Table 26, Table 27, or Table 28, or a combination thereof, in a plurality of patient samples of a selected tissue type; (b) determining a degree of similarity between (a) the differential gene expression profile data in the patient samples; and (b) a differential gene profile specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, produced by a therapy which has been shown to be efficacious in treatment of the at least one autism spectrum disorder; wherein a high degree of similarity of the differential gene expression profile data is indicative that the treatment is effective.

In yet another aspect of the invention, a method is provided for determining a gene profile indicative of administration of a therapeutic treatment to a subject with at least one autism spectrum disorder comprising (a) preparing samples of control and experimental cDNA, wherein the experimental cDNA is generated from a nucleic acid sample isolated from a subject who has received the therapeutic treatment; (b) preparing one or more microarrays comprising a plurality of different oligonucleotides, wherein the oligonucleotides are specific to genes associated with an autism spectrum disorder; (c) applying the prepared samples to the one or more microarrays to allow hybridization between the oligonucleotides and the control and experimental cDNAs; (d) identifying the oligonucleotides on the microarray which display differential hybridization to the experimental cDNA relative to the control cDNA thereby determining a gene profile indicative for the administration of the therapeutic treatment to the subject with at least one autism spectrum disorder.

In another embodiment of the method of the present invention, the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

In yet another embodiment of the method of the present invention, the at least one autism spectrum disorder neurological condition comprises autistic disorder, pervasive developmental disorder-not otherwise specified (PDD-NOS), including atypical autism, Asperger's Disorder, or a combination thereof.

In yet another aspect of the invention, a method is provided for conducting drug discovery comprising (a) generating a database of gene profile data representative of the genetic expression response of at least one selected neuronal tissue type from a subject that was subjected to at least one of a plurality of behavioral therapies and that has undergone a selected physiological change since commencement of the behavioral therapy; (b) administering small molecule test agents to untreated subjects to obtain gene expression profile data associated with administration of the agents and comparing the obtained data with the one or more selected gene profiles; (c) selecting test agents that induce gene profiles similar to gene profiles obtainable by administration of behavioral therapy; (d) conducting therapeutic profiling of the selected test compound(s), or analogs thereof, for efficacy and toxicity in subjects; and (e) identifying a pharmaceutical preparation including one or more agents identified in step (d) as having an acceptable therapeutic and/or toxicity profile.

In another embodiment of the method of the present invention, the behavioral therapy comprises applied behavior analysis (ABA) intervention methods, dietary changes, exercise, massage therapy, group therapy, talk therapy, play therapy, conditioning, or alternative therapies such as sensory integration and auditory integration therapies.

In yet another embodiment of the method of the present invention, the selected physiological change includes one or more improvements in social interaction, language abilities, restricted interests, repetitive behaviors, sleep disorders, seizures, gastrointestinal, hepatic, and mitochondrial function, neural inflammation, or a combination thereof.

In yet another embodiment of the method of the present invention, prior to administration of behavioral therapy, the subject shows at least one symptom of a psychological or physiological abnormality.

In yet another embodiment of the method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

In yet another aspect of the invention, a kit is provided for identifying a compound for treating at least one autism spectrum disorder comprising (a) a database having information stored therein one or more differential gene expression profiles specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, of subjects that have been subjected to at least one of a plurality of selected autism spectrum disorder neurological therapies and wherein the subject has undergone a desired physiological change; and (b) a computer program for comparing gene expression profile data obtained from assays wherein a test compound is administered to a subject with the database and providing information representative of a measure of similarity between the gene expression profile data and one or more stored gene profiles.

In yet another aspect of the invention, a computer-implemented method is provided for determining a gene profile for at least one autism spectrum disorder wherein the method comprises the steps of: (a) generating a database of gene profile data representative of the differential gene expression profiles specific for genes that have been determined to have increased or decreased expression in subjects with an autism spectrum disorder into a form suitable for computer-based analysis; and (b) analyzing the compiled data, wherein the analyzing comprises identifying gene networks from a number of upregulated pathway genes and/or downregulated pathway genes, wherein the pathway genes include those genes that have been identified as associating with severity of autism or an autism spectrum disorder, wherein said genes comprise at least five different genes set out in listed in Table 3, Table 7, Table 8, Table 9, Table 10, Table 18, Table 19, Table 21, Table 22, Table 23, Table 25, Table 26, Table 27, or Table 28, or a combination thereof.

In yet another aspect of the invention, a computer-readable medium is provided on which is encoded programming code for analyzing autism spectrum disorder differential gene expression from a plurality of data points comprising a gene expression profile of differentially expressed genes, wherein said differential gene expression profile is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

In yet another aspect of the invention, each of the gene chip compositions and methods of use thereof, kits and computer readable mediums specifically provided for supra (and infra) may also be, without any limitation, made and/or practiced with at least one, two, three, four, or five or more of any of the genes described in any one or more of Tables 1-28 as shown infra.

In yet another embodiment of the invention, in each of the screening methods, gene profiling methods, phenotype distinguishing methods, drug discovery methods, compound efficacy testing methods, computer-implemented methods for determining a gene profile, and kits described supra, the differential gene expression profile is specific for at least twenty different genes set out in Table 3, Table 7, Table 8, Table 9, Table 10, Table 18, Table 19, Table 21, Table 22, Table 23, Table 25, Table 26, Table 27, or Table 28, or a combination thereof.

### BRIEF DESCRIPTION OF THE DRAWINGS

The foregoing and other aspects and advantages of the invention will be appreciated more fully from the following further description thereof, with reference to the accompanying drawings wherein:

FIG. 1 Average ADIR scores for specific items within functional categories for the 4 different subgroups of individuals whose LCL were analyzed for gene expression profiles. A) Average item scores for language skills, social development, interests and behaviors and savant skills for the severely language impaired (red), mild ASD (blue), savant (yellow), and language impaired+savant (orange) groups. B) Average item scores for nonverbal communication, play skills, physical sensitivities and mannerisms, and aggression for the 4 phenotypic groups.

FIG. 2 A) Overlap of neurologically relevant differentially expressed genes in both severe language impaired (L) and mild (M) ASD subgroups. Pathway Studio 5 network prediction software was used to create a network of overlapping differentially expressed which are functionally related. It is of interest that the network entities involve not only neurological functions, but also functions and disorders, such as hypercholesterolemia, adrenal gland dysfunction, and diabetes mellitus, which may be responsible for the additional physiological symptoms manifested to varying extents by individuals with ASD. B) Confirmation of 5 of the overlapping genes by qRT-PCR analyses on 5 representative samples from the L and M subgroups.

FIG. 3 Differential gene expression (relative to the average of the control group) of the 13 genes involved in circadian rhythm across 31 individuals in the language subgroup of ASD.

FIG. 4 Gene network showing relationships between significantly differentially expressed genes (FDR<13.5%) between autistic and non-autistic siblings. The expression cutoff was set at a mean log 2(ratio) of ≧±0.29 prior to analysis with IPA.

FIG. 5 Gene network constructed by Pathway Studio 5 analysis of 11 RT-qPCR-confirmed differentially expressed genes. The color coding of the entities within this relational gene/molecular network are as follows: Red—genes that show increased expression in autistic individuals on average relative to controls; Green—genes that show decreased expression in autistic individuals on average relative to controls; Blue—small molecules including steroid and stress hormones, neurotransmitters, and other metabolites; Pink—other genes that link the differentially expressed genes together in this network; Yellow—cell processes; Lavender—disorders; Orange—functional class; Turquoise.

FIG. 6 A bionetwork that shows the relationships and interactions among SCARB1, BZRP, and SRD5A1 at the gene, protein, and metabolite levels. Briefly, SCARB1 is responsible for the uptake of cholesterol into cells while BZRP (aka. TSPO) transports cholesterol from the cytoplasm to the mitochondrial matrix where steroidogenesis takes place. SRD5A1, in turn, converts testosterone to 5-α-dihydrotestosterone (DHT), a more potent form of the male hormone. We propose that increases in the gene expression of at least some of these genes may lead to an overall increase in the production of androgens. It is also of interest that bile acid synthesis is linked to this same pathway, thereby suggesting that altered expression of these genes in ASD may lead to disturbances of bile acid synthesis in some tissues as well.

### DETAILED DESCRIPTION OF THE INVENTION

The invention disclosed herein provides methods and compositions for diagnosis and treatment of neurological conditions. In particular, the invention provides microarray technology to diagnose and treat autism spectrum disorders. The invention relates, in part, to sets of genetic markers whose expression patterns correlate with therapeutic treatments of neurological, and in particular, autism spectrum disorders.

The invention provides not only methods of identifying gene profiles for neurological conditions, but also methods of using such gene profiles in order to select particular therapeutic compounds useful in the prevention and treatment of such neurological conditions. The invention further relates to the application of gene profiles for the identification of therapeutic targets, and related pharmaceutical methods and kits.

The systems and methods described herein include microarray systems including gene chips and arrays of nucleotide sequences for detecting gene profiles of neurological conditions, and in particular, autism spectrum disorder conditions. The systems and methods described herein provide microarrays that have a plurality of oligonucleotide primers immobilized thereon and have specificity for genes associated with neurological conditions, and in particular, autism spectrum disorder conditions.

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof.

### DEFINITIONS

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The terms “microarray,” “GeneChip,” “genome chip,” and “biochip,” as used herein refer to an ordered arrangement of hybridizeable array elements. The array elements are arranged so that there are preferably at least one or more different array elements on a substrate surface, such as paper, nylon or other type of membrane, filter, chip, glass slide, or any other suitable solid support. The hybridization signal from each of the array elements is individually distinguishable.

The terms “complementary” or “complementarity” as used herein refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “compound” and “test compound” refer to any chemical entity, pharmaceutical, drug, and the like that can be used to treat or prevent a disease, illness, conditions, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of cancer. Examples of test compounds include, but are not limited to peptides, polypeptides, synthetic organic molecules, naturally occurring organic molecules, nucleic acid molecules, and combinations thereof.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

As used herein, the term “subject” refers to a cell, tissue, or organism, human or non-human, whether in vivo, ex vivo or in vitro, under observation.

As used herein, the term “increased expression” refers to the level of a gene expression product that is made higher and/or the activity of the gene expression product that is enhanced. Preferably, the increase is by at least 1.22-fold, 1.5-fold, more preferably the increase is at least 2-fold, 5-fold, or 10-fold, and most preferably, the increase is at least 20-fold, relative to a control.

As used herein, the term “decreased expression” refers to the level of a gene expression product that is made lower and/or the activity of the gene expression product that is lowered. Preferably, the decrease is at least 25%, more preferably, the decrease is at least 50%, 60%, 70%, 80%, or 90% and most preferably, the decrease is at least one-fold, relative to a control.

As used herein, the term “gene profile” refers to an experimentally verified subset of values associated with the expression level of a set of gene products from informative genes which allows the identification of a biological condition, an agent and/or its biological mechanism of action, or a physiological process.

As used herein, the term “gene expression profile” refers to the level or amount of gene expression of particular genes, for example, informative genes, as assessed by methods described herein. The gene expression profile can comprise data for one or more informative genes and can be measured at a single time point or over a period of time. For example, the gene expression profile can be determined using a single informative gene, or it can be determined using two or more informative genes, three or more informative genes, five or more informative genes, ten or more informative genes, twenty-five or more informative genes, or fifty or more informative genes. A gene expression profile may include expression levels of genes that are not informative, as well as informative genes. Phenotype classification (e.g., the presence or absence of a neurological disorder) can be made by comparing the gene expression profile of the sample with respect to one or more informative genes with one or more gene expression profiles (e.g., in a database). Using the methods described herein, expression of numerous genes can be measured simultaneously. The assessment of numerous genes provides for a more accurate evaluation of the sample because there are more genes that can assist in classifying the sample. A gene expression profile may involve only those genes that are increased in expression in a sample, only those genes that are decreased in expression in a sample, or a combination of genes that are increased and decreased in expression in a sample.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “neurological condition” or “neurological disorder” is used herein to mean mental, emotional, or behavioral abnormalities. These include but are not limited to autism spectrum disorder conditions including autism, asperger's disorder, bipolar disorder I or II, schizophrenia, schizoaffective disorder, psychosis, depression, stimulant abuse, alcoholism, panic disorder, generalized anxiety disorder, attention deficit disorder, post-traumatic stress disorder, Parkinson's disease, or a combination thereof.

Gene Chips

One aspect of the invention provides gene chips. Gene chips, also called “biochips” or “arrays” or “microarrays” are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided that such techniques are amenable to and compatible with the deposition and screening of polynucleotide sequences. Microarrays are particularly desirable for their virtues of high sample throughput and low cost for generating profiles and other data.

One specific aspect of the invention provides a gene chip having a plurality of different oligonucleotides having specificity for genes associated with neurological conditions, and in particular, autism spectrum disorder conditions including pervasive developmental disorder-not otherwise specified (PDD-NOS), including atypical autism, Asperger's Disorder, or a combination thereof. In a related embodiment, the invention provides a gene chip having a plurality of different oligonucleotides having specificity for genes whose expression level changes in a subject who is afflicted with neurological conditions, and in particular, autism spectrum disorder conditions including pervasive developmental disorder-not otherwise specified (PDD-NOS), including atypical autism, Asperger's Disorder, or a combination thereof when the subject responds favorably to a therapeutic treatment that is intended to treat the neurological condition.

In one embodiment of the gene chips provided herein, the oligonucleotides on the gene chip comprise oligonucleotides that are specific for the genes set out in Tables 1-3, or combinations thereof. In another embodiment, the gene chip has oligonucleotides specific for the genes associated with autism spectrum disorder conditions including pervasive developmental disorder-not otherwise specified (PDD-NOS), including atypical autism, Asperger's Disorder, or a combination thereof.

In another specific embodiment, the gene chip has at least one oligonucleotide specific for genes associated with the cellular response to androgens. In another specific embodiment, the gene chip has at least one oligonucleotide specific for genes associated with the cellular response to androgens including Gen Bank Accession Numbers AA907052, AI076295 (MEMO1 locus), H25019 (ZZZ3 locus), H97875, R11217, or any combination thereof.

In another specific embodiment, the gene chip has at least one oligonucleotide specific for genes associated with circadian rhythm. In another specific embodiment, the gene chip has at least one oligonucleotide specific for the circadian rhythm associated genes AANAT, BHLHB2, BHLHB3, CLOCK, CREM, CRY1, DPYD, MAPK1, NFIL3, NPAS2, NR1D1, PER1, PER3, PTGDS, RORA, or any combination thereof.

In another specific embodiment, the gene chip has at least one oligonucleotide specific for genes associated with WNT signaling, axon guidance, regulation of the cytoskeleton; Type II Diabetes Mellitus, insulin signaling pathways, cholesterol metabolism, and steroid hormone biosynthesis pathways, nervous system development, synaptic transmission or plasticity, myelination, long-term potentiation, neuron toxicity, embryonic development, regulation of actin networks, digestion, liver toxicity (hepatic stellate cell activation, fibrosis, and cholestasis), inflammation, oxidative stress, epilepsy, apoptosis, cell survival, differentiation, the unfolded protein response, endocrine function, circadian rhythm, cholesterol metabolism or a combination thereof.

In another embodiment, the gene chip comprises oligonucleotide probes specific for genes associated with apoptosis and inflammation, as well as many neurological and metabolic processes commonly associated with ASD, such as myelination, neuron plasticity, synaptic transmission, and hypercholesterolemia. In one embodiment, the gene chip comprises oligonucleotides specific for ITGAM, NFKB1, RHOA, SLIT2, MBD2, MECP2, or a combination thereof.

In another specific embodiment of the gene chips provided herein, the gene chip comprises at least 3, 5, 10, 15, 20 or 25 of the probes are derived from oligonucleotides that are specific for the genes set out in any one of Tables 1-3, or 28, or combinations thereof. In a related embodiment, at least 50% of the probes on the gene chip are derived from oligonucleotides that are specific for the genes present in any one of Tables 1-3, or 28. In a related embodiment, at least 70%, 80%, 90%, 95% or 98% of the probes on the gene chip are derived from oligonucleotides that are specific for the genes present in any one of Tables 1-3, or 28, or combinations thereof.

The invention further provides a gene chip for distinguishing cell samples from individuals having a positive prognosis and cell samples from individuals having a negative prognosis, wherein prognosis refers to the progression of disease or prognosis for successful treatment by a given treatment regimen or agent, comprising a positionally-addressable array of polynucleotide probes bound to a support, said polynucleotide probes comprising a plurality of polynucleotide probes of different nucleotide sequences, each of said different nucleotide sequences comprising a sequence complementary and hybridizable to a different, said plurality consisting of at least 5 of the genes corresponding to the genes listed in Tables 1-3, or 28.

In some embodiments of the gene chips, processes, methods and kits provided by the invention, the neurological condition is selected from the group consisting of autism spectrum disorders, autism, atypical autism, pervasive developmental disorder-not otherwise specified (PDD-NOS), asperger's disorder, Rett's syndrome, allodynia, catalepsy, hypernocieption, Parkinson's disease, parkinsonism, cognitive impairments, age-associated memory impairments, cognitive impairments, dementia associated with neurologic and/or neurological conditions, allodynia, catalepsy, hypernocieption, and epilepsy, brain tumors, brain lesions, multiple sclerosis, Down's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, Asperger's Disorder, bipolar manic-depressive disorder, ischemia, alcohol addiction, drug addiction, obsessive compulsive disorders, Pick's disease and Binswanger's disease.

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods and gene chips of the invention may be immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or about 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to one aspect of the invention, the microarray is an array (i.e., a matrix) in which each position represents one of the markers or gene biomarkers as described herein. For example, each position can contain a DNA or DNA analogue based on genomic DNA to which a particular RNA or cDNA transcribed from that genetic marker or biomarker can specifically hybridize. The DNA or DNA analogue can be, for example, a synthetic oligomer or a gene fragment. In one embodiment, probes representing each of the genes or biomarkers on Tables 1-3, or 28 are present on the array.

As noted above, the “probe” to which a particular polynucleotide molecule specifically hybridizes according to the invention contains a complementary polynucleotide sequence. In one embodiment, the probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR: Protocols: A Guide to Methods and Applications, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083). Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the cDNA molecules, and negative control probes, e.g., probes known to not be complementary and hybridizable to sequences in the cDNA molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes may be attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodoret al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller. In one embodiment, the arrays of the present invention are prepared by synthesizing polynucleotide probes on a support. In such an embodiment, polynucleotide probes are attached to the support covalently at either the 3′ or the 5′ end of the polynucleotide.

In a one embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA ARRAYS IN GENETIC ENGINEERING, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

Methods of Determining Gene Profiles

One aspect of the invention provides methods for determining a gene profile for a specific neurological disorder or neurological condition, such as autism spectrum disorder conditions including autistic disorder, pervasive developmental disorder-not otherwise specified (PDD-NOS), including atypical autism, Asperger's Disorder. Furthermore, the systems and methods described herein may be employed to generate gene profiles for diseases or disorders of interest. This expression data may be analyzed independently to determine a gene profile of interest, or combined with the existing biological data stored in a plurality of different types of databases. Statistical analyses may be applied as well as machine learning techniques that are used to discover trends and patterns in the underlying data. These techniques include clustering methods, which can be used for example to organize microarray expression data.

One specific aspect of the invention provides a method for determining a gene profile for a neurological condition, comprising (i) preparing samples of control and experimental cDNA, wherein the experimental cDNA is generated from a nucleic acid sample isolated from a subject suspected of being afflicted with the neurological condition; (ii) preparing one or more microarrays comprising a plurality of different oligonucleotides having specificity for genes associated with the neurological condition; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; and (vi) identifying a set of genes from the oligonucleotides identified in step (v) thereby determining a gene profile for the neurological condition.

In a preferred embodiment, the neurological condition is an autism spectrum disorder condition including autistic disorder, pervasive developmental disorder-not otherwise specified (PDD-NOS), including atypical autism, Asperger's Disorder, or a combination thereof. In another embodiment, the neurological condition is selected from the group consisting of autism spectrum disorder conditions including autistic disorder, pervasive developmental disorder-not otherwise specified (PDD-NOS), including atypical autism, Asperger's Disorder, Rett's syndrome, Parkinson's disease, parkinsonism, cognitive impairments, age-associated memory impairments, cognitive impairments, dementia associated with neurologic and/or neurological conditions, allodynia, catalepsy, hypernocieption, and epilepsy, brain tumors, brain lesions, multiple sclerosis, Down's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, bipolar manic-depressive disorder, ischemia, alcohol addiction, drug addiction, obsessive compulsive disorders, Pick's disease and Binswanger's disease.

In another embodiment, the samples of experimental cDNA may be isolated from a subject or group of subjects suspected of being afflicted or afflicted with one or more neurological conditions. Control cDNA may be derived from a nucleic acid sample of a subject or group of subjects which are not afflicted with the neurological conditions that the subjects from which the experimental cDNA was derived. In another embodiment, the subjects from which the experimental and control samples are derived may both be suspected of being afflicted or afflicted with the condition, but the severity of the condition or a treatment plan in the two subject groups may differ.

A related aspect of the invention provides a method of determining a gene profile for the administration of a therapeutic treatment to a subject. Such methods are useful to detect the gene expression changes that accompany the underlying therapeutic treatments. A gene profile for such genetic changes may be used to determine if a second therapeutic treatment is expected to have the same effect, by comparing the gene expression profile of the second treatment to the gene profile of the first.

Accordingly, one specific aspect of the invention provides a method of determining a gene profile indicative for the administration of a therapeutic treatment to a subject, the method comprising (i) preparing samples of control and experimental cDNA, wherein the experimental cDNA is generated from a nucleic acid sample isolated from a subject who has received or is receiving the therapeutic treatment; (ii) preparing one or more microarrays comprising a plurality of different oligonucleotides wherein the oligonucleotides are specific to genes associated with an autism spectrum disorder; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; (vi) identifying a set of genes associated with an autism spectrum disorder from the oligonucleotides identified in step (v) thereby determining a gene profile for the administration of the therapeutic treatment to the subject.

In yet another aspect of the invention, a method is provided for determining a gene profile for at least one autism spectrum disorder, comprising (a) preparing samples of control and experimental cDNA, wherein the experimental cDNA is generated from a nucleic acid sample isolated from a subject suspected of being afflicted with the at least one autism spectrum disorder and the control cDNA is generated from a nucleic acid sample isolated from a healthy individual; (b) preparing one or more microarrays comprising a plurality of different oligonucleotides having specificity for genes associated with the at least one autism spectrum disorder; (c) applying the prepared samples to the one or more microarrays to allow hybridization between the oligonucleotides and the control cDNA and the oligonucleotide and the experimental cDNAs; (d) identifying the oligonucleotides on the microarray which display differential hybridization to the experimental cDNA relative to the control cDNA thereby determining a gene profile for the at least one autism spectrum disorder.

In yet another aspect of the invention, a method is provided for distinguishing between different phenotypes of an autism spectrum disorder comprising severely language impaired (L), mildly affected (M), or “savants” (S) comprising (a) preparing samples of control and experimental cDNA, wherein the experimental cDNA is generated from a nucleic acid sample isolated from a subject suspected of being afflicted with at least one phenotype comprising the severely language impaired (L), mildly affected (M), or “savants” (S); (b) preparing one or more microarrays comprising a plurality of different oligonucleotides having specificity for genes associated with the at least one phenotype; (c) applying the prepared samples to the one or more microarrays to allow hybridization between the oligonucleotides and the control and experimental cDNAs; (d) identifying the oligonucleotides on the microarray which display differential hybridization to the experimental cDNA relative to the control cDNA thereby determining a gene profile for distinguishing among the different phenotypes of autism spectrum disorder.

In yet another embodiment of the screening method of the present invention, the method distinguishes between different variants of autism spectrum disorder comprising a lower severity scores across all ADIR items, an intermediate severity across all ADIR items, a higher severity scores on spoken language items on the ADIR, a higher frequency of savant skills, and a severe language impairment, or a combination thereof.

In one embodiment of the methods for determining a gene profile for the administration of a therapeutic treatment, administration of therapeutic treatment results in a physiological change in the subject, such as a beneficial change. In a specific embodiment, the physiological change comprises one or more improvements in social interaction, language abilities, restricted interests, repetitive behaviors, sleep disorders, seizures, gastrointestinal, hepatic, and mitochondrial function, neural inflammation, or a combination thereof. In another embodiment, the control cDNA may be derived from the subject(s) prior to administration of the therapeutic treatment, or from a subject or group of subjects who do not receive the therapeutic treatment.

In another embodiment of the methods for determining a gene profile for the administration of a therapeutic treatment to a subject suspected of being afflicted with or afflicted with autism spectrum disorder conditions including autistic disorder, pervasive developmental disorder-not otherwise specified (PDD-NOS), including atypical autism, Asperger's Disorder, the therapeutic treatment may comprise a single procedure or it may comprise an aggregate of treatment procedures. In one embodiment, therapeutic treatment comprises a behavioral therapy, such as applied behavior analysis (ABA) intervention methods, dietary changes, exercise, massage therapy, group therapy, talk therapy, play therapy, conditioning, or alternative therapies such as sensory integration and auditory integration therapies. In another embodiment, the therapeutic treatment comprises administering to the subject a drug, such as an antidepressant or antipsychotic drug. In another embodiment, the subject is afflicted with a neurological condition other than autism spectrum disorder conditions including autistic disorder, pervasive developmental disorder-not otherwise specified (PDD-NOS), including atypical autism, Asperger's Disorder. Such condition may be one which the therapeutic treatment is intended to treat. In another embodiment, the subject is a healthy subject who is not afflicted with a neurological condition. In another embodiment, the therapeutic treatment is a treatment for the autism spectrum disorder neurological conditions including autistic disorder, pervasive developmental disorder-not otherwise specified (PDD-NOS), including atypical autism, Asperger's Disorder.

In another embodiment, the drug being administered in the single procedure or the aggregate of treatment procedures is a serotonergic antidepressant medication, such as one selected from the group consisting of citalopram, fluoxetine, fluvoxamine, paroxetine, or sertraline, or the drug is a catecholaminergic antidepressant medication, such as bupropion.

In another preferred embodiment of the ongoing methods, both the control cDNA and the experimental cDNA are derived from a nucleic acid sample isolated from the subject. Samples may be isolated from a mammal, such as a human. In a specific embodiment, the sample is isolated post-mortem from a human. Nucleic acid samples may be isolated from any tissue or bodily fluid, including blood, saliva, tears, cerebrospinal fluid, pericardial fluid, synovial fluid, amniotic fluid, semen, bile, ear wax, gastric acid, sweat, urine, or fluid drained from an edema. In a further specific embodiment, the nucleic acid sample is isolated from lymphoblastoid cells or lyphoblastoid cell lines (LCL) derived from blood cells of subjects. In some embodiments of the ongoing methods, the sample is isolated from a neuronal tissue or a combination of tissue types, such as olfactory bulb cells, cerebrospinal fluid, hypothalamus, amygdala, pituitary, spinal cord, brainstem, cerebellum, cortex, frontal cortex, hippocampus, choroid plexus, striatum, and thalamus.

In one embodiment of the ongoing methods, the microarray is any one of the microarrays, or gene chips, described herein. In a preferred embodiment, the oligonucleotides on the microarray comprise those specific to genes selected from Table 3, Table 7, Table 8, Table 9, Table 10, Table 18, Table 19, Table 21, Table 22, Table 23, Table 25, Table 26, Table 27, or Table 28, or a combination thereof. In a specific embodiment, the oligonucleotides of the microarray are specific to genes associated with circadian rhythm, WNT signaling, axon guidance, regulation of the cytoskeleton, and dendrite branching, Type II Diabetes Mellitus, insulin signaling pathways, cholesterol metabolism and steroid hormone biosynthesis pathways as described supra. In a preferred embodiment, at least 10%, 20%, 30%, 40%, 50%, 60%, 70%, 80%, 90%, 95%, or 99% of the genes on the microarray are specific to genes selected from Table 3, Table 7, Table 8, Table 9, Table 10, Table 18, Table 19, Table 21, Table 22, Table 23, Table 25, Table 26, Table 27, or Table 28, or a combination thereof.

In another embodiment of the ongoing methods, the control cDNA and the experimental cDNAs are hydridized to the same microarray, while in another embodiment they are hybridized to separate but substantially identical microarrays. If the same microarray is used, the cDNA samples may be labeled using fluorescent compounds having different emission wavelengths such that the signals generated by each cDNA type may be distinguished from a single microarray.

In yet another embodiment of the ongoing methods, the control and experimental cDNA is isolated from one or more subjects. In one embodiment, the control cDNA and experimental cDNA are isolated each from at least 3, 5, 10, 15 or 20 subjects. The cDNAs from each subject may be hybridized to the microarrays separately, or the control cDNAs, or the experimental cDNAs, may be pooled together, such that, for example, an experimental cDNA sample is derived from multiple subjects. In preferred embodiments, the subjects are mammals, such as rodents, primates or humans.

In one embodiment of the ongoing methods, the set of genes in the gene profile comprise genes which have a differential expression in the experimental cDNA relative to the control cDNA. Differential expression may refer to a lower expression level or to a higher expression. In preferred embodiments, the difference in expression level is statistically significant for each gene, or marker, on the set. In preferred embodiments, the difference in expression is at least 10%, 20%, 30%, 40%, 50%, 60%, 70%, 80%, 90%, 100%, 150%, 200%, 300%, 400%, or 500% greater in the experimental cDNA than in the control cDNA, or vice versa. In another preferred embodiment, the difference in expression is at least about 1.22-fold, 1.5-fold, 2-fold, 2.5-fold, 3-fold, 3.5-fold, 4-fold, 4.5-fold, 5-fold, 6-fold, 7-fold, 8-fold, 9-fold, 10-fold, 12-fold, 14-fold, 16-fold, 18-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold greater (or intermediate ranges thereof as another example) in the experimental cDNA than in the control cDNA, or vice versa A gene profile may comprise all the genes which are differentially expressed between the control and experimental cDNAs or it may comprise a subset of those genes. In some embodiments, the gene profile comprises at least 1%, 2%, 3%, 4%, 5%, 10%, 20%, 30%, 40%, 50%, 60%, 70%, 80%, 90%, 95%, 98%, 99% or 100% (or intermediate ranges thereof as another example) of the genes having differential expression. Genes showing large, reproducible changes in expression between the two samples are preferred in some embodiments. In preferred embodiments, the gene profile further comprises a subset of values associated with the expression level of each of the genes in the profile, such that gene profile allows the identification of a biological and/or pathological condition, an agent and/or its biological mechanism of action, or a physiological process.

The preparation of samples of control and experimental cDNA may be carried out using techniques known in the art. The cDNA molecules analyzed by the present invention may be from any clinically relevant source. In one embodiment, the cDNA is derived from RNA, including, but by no means limited to, total cellular RNA, poly(A).sup.+ messenger RNA (mRNA) or fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A).sup.+ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from a sample of cells of the various tissue types of interest, such as the lymphoblastoid cell or lymphoblastoid cell line derived therefrom or from the aforementioned neuronal tissue types, using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). Poly(A).sup.+ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl.sub.2, to generate fragments of RNA. In another embodiment, the polynucleotide molecules analyzed by the invention comprise cDNA, or PCR products of amplified RNA or cDNA. CDNA molecules that are poorly expressed in particular cells may be enriched using normalization techniques (Bonaldo et al., 1996, Genome Res. 6:791-806).

The cDNAs may be detectably labeled at one or more nucleotides. Any method known in the art may be used to detectably label the cDNAs. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the cDNAs. Alternatively, random primers may be used in conjunction with PCR methods or T7 promoter-based in vitro transcription methods in order to amplify the cDNAs.

In one embodiment, the detectable label is a luminescent label. For example, fluorescent labels, bioluminescent labels, chemiluminescent labels, and colorimetric labels may be used in the present invention. In one preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, the experimental cDNA are labeled differentially from the control cDNA, especially if both the cDNA types are hybridized to the same microarray. The control cDNA can comprise target polynucleotide molecules from normal individuals (i.e., those not afflicted with the neurological disorder or subjects who have not undergone to therapeutic treatment). In one preferred embodiment, the control cDNA comprises target polynucleotide molecules pooled from samples from normal individuals. In one embodiment of the methods for generating a gene profile of a therapeutic treatment, the control cDNA is derived from the same subject, but taken at a different time point, such as before, during or after the therapeutic treatment.

Nucleic acid hybridization and wash conditions are chosen so that the cDNA molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the cDNA molecules. Arrays containing single-stranded probe DNA (e.g., synthetic oligodeoxyribonucleic acids) may need to be denatured prior to contacting with the cDNA molecules, e.g., to remove hairpins or dimers which form due to self complementary sequences. Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5.times.SSC plus 0.2% SDS at 65° C. for four hours, followed by washes at 25° C. in low stringency wash buffer (1.times.SSC plus 0.2% SDS), followed by 10 minutes at 25° C. in higher stringency wash buffer (0.1.times.SSC plus 0.2% SDS) (Schena et al., Proc. Natl. Acad. Sci. U.S.A. 93:10614, (1993)). Useful hybridization conditions are also provided in, e.g., Tijessen, 1993, HYBRIDIZATION WITH NUCLEIC ACID PROBES, Elsevier Science Publishers B. V.; and Kricka, 1992, NONISOTOPIC DNA PROBE TECHNIQUES, Academic Press, San Diego, Calif. Hybridization conditions may include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

When fluorescently labeled cDNAs are used in the aforementioned methods, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In one preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Signals may be recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different neurological conditions.

In another embodiment of the present invention, changes in gene expression may be assayed in at least one cell of a subject by measuring transcriptional initiation, transcript stability, translation of transcript into protein product, protein stability, or a combination thereof. The gene, transcript, or polypeptide can be assayed by techniques such as in vitro transcription, in vitro translation, quantitative nuclease protection assay (qNPA) analysis, Western analysis, focused gene chip analysis, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift assay (EMSA), radioimmunoassay (RIA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, .beta.-galactosidase (LacZ), chloramphenicol acetyltransferase (CAT), .beta.-glucoronidase (GUS), bacterial/insect/marine invertebrate luciferases (LUC), green and red fluorescent proteins (GFP and RFP, respectively), horseradish peroxidase (HRP), .beta.-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine (SEQ ID NO: 45), maltose binding protein) for which cognate antibodies or affinity resins are available.

In another embodiment, the gene, transcript, or polypeptide can be assayed by use systems employing expression vectors. An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian gene expressed in the microvasculature, especially endothelial cells (e.g., ICAM-2, tie), or a virus (e.g., adenovirus, adeno-associated virus, cytomegalovirus, fowlpox virus, herpes simplex virus, lentivirus, Moloney leukemia virus, mouse mammary tumor virus, Rous sarcoma virus, SV40 virus, vaccinia virus), as well as regions suitable for genetic manipulation (e.g., selectable marker, linker with multiple recognition sites for restriction endonucleases, promoter for in vitro transcription, primer annealing sites for in vitro replication). The expression vector may be associated with proteins and other nucleic acids in a carrier (e.g., packaged in a viral particle) or condensed with chemicals (e.g., cationic polymers) to target entry into a cell or tissue.

The expression vector further comprises a regulatory region for gene expression (e.g., promoter, enhancer, silencer, splice donor and acceptor sites, polyadenylation signal, cellular localization sequence). Transcription can be regulated by tetracyline or dimerized macrolides. The expression vector may be further comprised of one or more splice donor and acceptor sites within an expressed region; Kozak consensus sequence upstream of an expressed region for initiation of translation; and downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include mRNA degradation signal(s) if it is desired to express one or more of the downstream regions only under the inducing condition. An origin of replication may also be included that allows replication of the expression vector integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from any gene of interest, and be provided in either orientation with respect to the promoter; the expressed region in the antisense orientation will be useful for making cRNA and antisense polynucleotide. The gene may be derived from the host cell or organism, from the same species thereof, or designed de novo; but it is preferably of archael, bacterial, fungal, plant, or animal origin. The gene may have a physiological function of one or more nonexclusive classes: axon guidance, synaptic transmission or plasticity, myelination, long-term potentiation, neuron toxicity, embryonic development, regulation of actin networks, KEGG pathway, digestion, liver toxicity (hepatic stellate cell activation, fibrosis, and cholestasis), inflammation, oxidative stress, epilepsy, apoptosis, cell survival, differentiation, the unfolded protein response, Type II diabetes and insulin signaling, endocrine function, circadian rhythm, cholesterol metabolism and the steroidogenesis pathway, adhesion proteins; steroids, cytokines, hormones, and other regulators of cell growth, mitosis, meiosis, apoptosis, differentiation, circadian rhythm, or development; soluble or membrane receptors for such factors; adhesion molecules; cell-surface receptors and ligands thereof; cytoskeletal and extracellular matrix proteins; cluster differentiation (CD) antigens, antibody and T-cell antigen receptor chains, histocompatibility antigens, and other factors mediating specific recognition in immunity; chemokines, receptors thereof, and other factors involved in inflammation; enzymes producing lipid mediators of inflammation and regulators thereof; clotting and complement factors; ion channels and pumps; transporters and binding proteins; neurotransmitters, neurotrophic factors, and receptors thereof; cell cycle regulators, oncogenes, and tumor suppressors; other transducers or components of signaling pathways; proteases and inhibitors thereof; catabolic or metabolic enzymes, and regulators thereof. Some genes produce alternative transcripts, encode subunits that are assembled as homopolymers or heteropolymers, or produce propeptides that are activated by protease cleavage. The expressed region may encode a translational fusion; open reading frames of the regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

IV Methods of Identifying or Characterizing Therapeutic Compounds

Another aspect of the invention is identification or screening of chemical or genetic compounds, derivatives thereof, and compositions including same that are effective in treatment of neurological diseases or disorders and individuals at risk thereof. The amount that is administered to an individual in need of therapy or prophylaxis, its formulation, and the timing and route of delivery is effective to reduce the number or severity of symptoms, to slow or limit progression of symptoms, to inhibit expression of one or more of the aforementioned genes that are transcribed at a higher level in neurological disease, to activate expression of one or more of the aforementioned genes that are transcribed at a lower level in neurological disease, or any combination thereof. Determination of such amounts, formulations, and timing and route of drug delivery is within the skill of persons conducting in vitro assays, in vivo studies of animal models, and human clinical trials.

A screening method may comprise administering a candidate compound to an organism or incubating a candidate compound with a cell, and then determining whether or not gene expression is modulated. Such modulation may be an increase or decrease in activity that partially or fully compensates for a change that is associated with or may cause neurological disease. Gene expression may be increased at the level of rate of transcriptional initiation, rate of transcriptional elongation, stability of transcript, translation of transcript, rate of translational initiation, rate of translational elongation, stability of protein, rate of protein folding, proportion of protein in active conformation, functional efficiency of protein (e.g., activation or repression of transcription), or combinations thereof. See, for example, U.S. Pat. Nos. 5,071,773 and 5,262,300. High-throughput screening assays are possible (e.g., by using parallel processing and/or robotics).

The screening method may comprise incubating a candidate compound with a cell containing a reporter construct, the reporter construct comprising transcription regulatory region covalently linked in a cis configuration to a downstream gene encoding an assayable product; and measuring production of the assayable product. A candidate compound which increases production of the assayable product would be identified as an agent which activates gene expression while a candidate compound which decreases production of the assayable product would be identified as an agent which inhibits gene expression. See, for example, U.S. Pat. Nos. 5,849,493 and 5,863,733.

The screening method may comprise measuring in vitro transcription from a reporter construct in the presence or absence of a candidate compound (the reporter construct comprising a transcription regulatory region) and then determining whether transcription is altered by the presence of the candidate compound. In vitro transcription may be assayed using a cell-free extract, partially purified fractions of the cell, purified transcription factors or RNA polymerase, or combinations thereof. See, for example, U.S. Pat. Nos. 5,453,362, 5,534,410, 5,563,036, 5,637,686, 5,708,158 and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

Another aspect of the invention provides methods of identifying, or predicting the efficacy of, test compounds. In particular, the invention provides methods of identifying compounds which mimic the effects of behavioral therapies. In still another aspect, the systems and methods described herein provide a method for predicting efficacy of a test compound for altering a behavioral response, by obtaining a database, e.g., as described in greater detail above, treating a test animal or human (e.g., a control animal or human that has not undergone other therapies, such as behavioral therapy) with the test compound, and comparing genetic expression data of tissue samples from the animal or human treated with the test compound to measure a degree of similarity with one or more gene profiles in said database. In certain embodiments, the untreated animal or human exhibits a psychological and/or behavioral abnormality possessed by the animals or humans used to generate the database prior to administration of the behavioral therapy.

In another aspect of the invention, a method is provided for predicting efficacy of a test compound for altering a behavioral response in a subject with at least one autism spectrum disorder comprising: (a) preparing a microarray comprising a plurality of different oligonucleotides, wherein the oligonucleotides are specific to genes associated with an autism spectrum disorder; (b) obtaining a gene profile representative of the gene expression profile of at least one sample of a selected tissue type from a subject subjected to each of at least one of a plurality of selected behavioral therapies which promote the behavioral response; (c) administering the test compound to the subject; and (d) comparing gene expression profile data in at least one sample of the selected tissue type from the subject treated with the test compound to determine a degree of similarity with one or more gene profiles associated with an autism spectrum disorder; wherein the predicted efficacy of the test compound for altering the behavioral response is correlated to said degree of similarity.

In another aspect, the systems and methods described herein relate to methods of identifying small molecules useful for treating neurological conditions.

For example, in another embodiment a database of gene profile data representative of the genetic expression response of a selected neuronal tissue type from an animal that was subjected to at least one of a plurality of behavioral therapies and that has undergone a selected physiological change since commencement of the behavioral therapy may be obtained. In an exemplary embodiment, subjects (e.g., subjects that display a preselected behavioral abnormality, such as an autism spectrum disorder neurological condition (including for example autistic disorder, pervasive developmental disorder-not otherwise specified (PDD-NOS), including atypical autism, Asperger's Disorder, Rett's syndrome), Parkinson's disease, parkinsonism, cognitive impairments, age-associated memory impairments, cognitive impairments, dementia associated with neurologic and/or neurological conditions, allodynia, catalepsy, hypernocieption, and epilepsy, brain tumors, brain lesions, multiple sclerosis, Down's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, bipolar manic-depressive disorder, ischemia, alcohol addiction, drug addiction, obsessive compulsive disorders, Pick's disease and Binswanger's disease or a combination thereof), are subjected to behavioral therapy (including, for example, applied behavior analysis (ABA) intervention methods, dietary changes, exercise, massage therapy, group therapy, talk therapy, play therapy, conditioning, or alternative therapies such as sensory integration and auditory integration therapies), and their tissues (including, for example, and not by way of limitation, lymphocytes, blood, or mucosal epithelial cells, brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue, and/or neurological tissues (including, for example, and not by way of limitation, olfactory bulb cells, cerebrospinal fluid, hypothalamus, amygdala, pituitary, nervous system, brainstem, cerebellum, cortex, frontal cortex, hippocampus, striatum, and thalamus) or a combination thereof are examined for physiological changes (one or more improvements in social interaction, language abilities, restricted interests, repetitive behaviors, sleep disorders, seizures, gastrointestinal, hepatic, and mitochondrial function, neural inflammation, or a combination thereof), and genetic expression responses are obtained for tissues that have undergone a desired change. In certain embodiments, the subjects are further selected for having undergone a desired change in behavior as well.

From such a database, biological targets for intervention can be identified, such as potential therapeutics (e.g., genes that are upregulated and thus may exert a beneficial effect on the physiology and/or behavior of the subject), potential receptor targets (e.g., receptors associated with upregulated proteins, the activation of which receptors may exert a beneficial effect on the physiology and/or behavior of the subject; or receptors associated with downregulated proteins, the inhibition of which may exert a beneficial effect on the physiology and/or behavior of the subject). In certain embodiments, one or more genes, the expression of which differs by a statistically significant amount in a treated subject as compared to an untreated control, may be selected as targets for intervention.

Small molecule test agents may then be screened in any of a number of assays to identify those with potential therapeutic applications. The term “small molecule” refers to a compound having a molecular weight less than about 2500 amu, preferably less than about 2000 amu, even more preferably less than about 1500 amu, still more preferably less than about 1000 amu, or most preferably less than about 750 amu. For example, subjects or tissue samples may be treated with such test agents to identify those that produce similar changes in expression of the targets, or produce similar gene profiles, as can be obtained by administration of behavioral therapy. Alternatively or additionally, such test agents may be screened against one or more target receptors to identify compounds that agonize or antagonize these receptors, singly or in combination, e.g., so as to reproduce or mimic the effect of behavioral therapy.

Compounds that induce a desired effect on targets, tissue, or subjects may then be selected for clinical development, and may be subjected to further testing, e.g., therapeutic profiling, such as testing for efficacy and toxicity in subjects. Analogs of selected compounds, e.g., compounds having similar cores but varying substituents and stereochemistry, may similarly be developed and tested. Agents that have acceptable characteristics for therapeutic use in humans or animals may be prepared as pharmaceutical preparations, e.g., with a pharmaceutically acceptable excipient (such as a non-pyrogenic or sterile excipient). Such agents may also be licensed to a manufacturer for development and/or commercialization, e.g., for manufacture and sale of a pharmaceutical preparation comprising said selected agent.

Accordingly, one aspect of the invention provides a method for predicting efficacy of a test compound for altering a behavioral response in a subject with at least one autism spectrum disorder comprising: (a) preparing a microarray comprising a plurality of different oligonucleotides, wherein the oligonucleotides are specific to genes associated with an autism spectrum disorder; (b) obtaining a gene profile representative of the gene expression profile of at least one sample of a selected tissue type from a subject subjected to each of at least one of a plurality of selected behavioral therapies which promote the behavioral response; (c) administering the test compound to the subject; and (d) comparing gene expression profile data in at least one sample of the selected tissue type from the subject treated with the test compound to determine a degree of similarity with one or more gene profiles associated with an autism spectrum disorder; wherein the predicted efficacy of the test compound for altering the behavioral response is correlated to said degree of similarity.

In one embodiment of the foregoing methods, step (a) comprises obtaining a gene profile representative of the gene expression profile of at least two samples of a selected tissue type referred to supra. In a related embodiment, step (a) comprises obtaining a gene profile data representative of the gene expression profile of at least three samples of a selected tissue referred to supra. In one embodiment in which the more than one sample of a selected tissue type referred to supra is used to determine a gene profile, the selected tissue types are different tissue types, whereas in other embodiments the tissue types are the same. For example, in an exemplary embodiment, a tissue type may be lymphoblastoid cells and a second tissue type olfactory bulb cells, such that the gene expression profile data generated from these two tissue samples in the treated subject may be compared to the gene profiles derived from the subjects subjected to the behavioral therapy. In other embodiments, gene profiles may be generated from multiple samples of the same tissue type from the same animal, such as blood samples taken at different intervals during the behavioral therapy.

In another embodiment of the foregoing methods, the gene profile is that shown in Table 3, Table 7, Table 8, Table 9, Table 10, Table 18, Table 19, Table 21, Table 22, Table 23, Table 25, Table 26, Table 27, or Table 28, or a combination thereof. In another embodiment, the gene profile comprises at least 5%, 10%, 20%, 30%, 40%, 50%, 60%, 70%, 80%, 90%, 95% or 98% of the genes shown in Table 3, Table 7, Table 8, Table 9, Table 10, Table 18, Table 19, Table 21, Table 22, Table 23, Table 25, Table 26, Table 27, or Table 28, or a combination thereof. In another embodiment, the gene profile comprises at least 5, 10, 15, 20, 25 or 30 of the genes listed in Table 3, Table 7, Table 8, Table 9, Table 10, Table 18, Table 19, Table 21, Table 22, Table 23, Table 25, Table 26, Table 27, or Table 28, or a combination thereof. In another embodiment of the foregoing methods, the gene profile comprises an increase in expression in ALS2CL, ASS, DAPK1, DDX26, DEXI, DTX1, NEB or a combination thereof. In another embodiment, the gene profile comprises a decrease in expression in CDC2L6, DST, EPC1, ITGAM, JAK1, MBD2, NFKB1, NR4A3, RHOA, SLC16A1, SLIT2, or a combination thereof.

In one embodiment of the foregoing methods, the selected tissue type comprises a neuronal tissue type, such as a neuronal tissue type selected from the group consisting of olfactory bulb cells, cerebrospinal fluid, hypothalamus, amygdala, pituitary, nervous system, brainstem, cerebellum, cortex, frontal cortex, hippocampus, striatum, and thalamus. In another embodiment, the selected tissue type is selected from the group consisting of brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth and tongue.

In one embodiment, the behavioral therapy comprises applied behavior analysis (ABA) intervention methods, dietary changes, exercise, massage therapy, group therapy, talk therapy, play therapy, conditioning, or alternative therapies such as sensory integration and auditory integration therapies.

In one embodiment of the foregoing methods, the test subject or animal is a human. In another embodiment, the animal is a non-human animal. Such non-human animals include vertebrates such as rodents, non-human primates, ovines, bovines, ruminants, lagomorphs, porcines, caprines, equines, canines, felines, ayes, etc. Preferred non-human animals are selected from the order Rodentia, most preferably mice. The term “order Rodentia” refers to rodents (i.e., placental mammals (Class Euthria) which include the family Muridae (rats and mice). In a specific embodiment, the test animal is a mammal, a primate, a rodent, a mouse, a rat, a guinea pig, a rabbit or a human.

The test compound may be administered to the subject or animal using any mode of administration, including, intravenous, subcutaneous, intramuscular, intrasternal, topical, liposome-mediate, rectal, intravaginal, opthalmic, intracranial, intraspinal or intraorbital. The test compound may be administered once or more than once as part of a treatment regimen. In some embodiments, additional test compounds or agents may be administered to the subject animal to ascertain the efficacy of the test compound or the combination of test compounds or agents. In some embodiments, a gene expression profile may also be obtained from the subject or animal prior to treatment with the test agent. In such embodiments, the efficacy of the test agent may be determined by comparing the gene expression profile of the subject or animal after treatment with the compound with (a) the gene expression profile prior to treatment with the compound and (b) to the gene profile for the behavioral therapy. For example, if the test compound causes the gene expression profile to approach that of said gene profile, the test compound may be predicted to be efficacious.

It is understood by one skilled in the art that the order of steps (a) and (b) in the foregoing methods may be interchanged i.e. the subject or animal may be treated with the compound prior to obtaining the genetic data profile for the behavior therapy. Accordingly, the invention also provides a method wherein step (b) is performed prior to step (a).

When comparing the gene expression profile data in at least one sample of the selected tissue type from the subject or animal treated with the test compound to determine a degree of similarity with one or more gene profiles, any number of statistical methods known to one skilled in the art may be used. In some embodiments, a gene profile may be obtained from samples of a test subject or animal prior to the administration of the test compound or from a control subject or animal to generate a control gene profile for each of the tissue types of interest. In such embodiments, the gene expression profile from the tissue types of the test subjects or animal(s) may be compared to both the control gene profiles and the gene profiles resulting from the behavioral therapy to determine to which of these profiles the gene expression profile is most similar. If they are more similar to the control gene profile, the test compound may be considered to less efficacious, whereas if it is more similar to the gene profile of the behavioral therapy then the compound is considered more efficacious.

In one variation of the ongoing methods, more than one test compound may be administered to the test subject or animal, such that the efficacy of a combination of test compounds is tested. In another variation, rather than using, or in addition to using, a test compound, a nonchemical test agent is also applied to the subject or animal, such as for example, and not by way of limitation, temperature, humidity, sunlight exposure or any other environmental factor. In yet another environment, the subject or animal is subjected to an invasive or noninvasive surgical procedure, in lieu or in addition to the test compound. In such embodiments, the efficacy of the surgical procedure may be ascertained.

In still yet another aspect, the systems and methods described herein relate to a kit for identifying a compound for treating a behavioral disorder, comprising a database, e.g., as described in greater detail above, and a computer program for comparing gene expression profile data obtained from assays wherein a test compound is administered to an untreated subject or animal with gene expression profile data in the database and identifying similarity between the gene expression profile data from the assays and one or more stored profiles.

In yet another aspect of the invention, the systems and methods described herein relate a kit is provided for identifying a compound for treating at least one autism spectrum disorder comprising (a) a database having information stored therein one or more differential gene expression profiles specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, of subjects that have been subjected to at least one of a plurality of selected autism spectrum disorder neurological therapies and wherein the subject has undergone a desired physiological change; and (b) a computer program for comparing gene expression profile data obtained from assays wherein a test compound is administered to a subject with the database and providing information representative of a measure of similarity between the gene expression profile data and one or more stored gene profiles.

Another aspect of the invention provides a method of assessing treatment efficacy in an individual having a neurological disorder comprising determining the expression level of one or more of the aforementioned informative genes in Table 3, Table 7, Table 8, Table 9, Table 10, Table 18, Table 19, Table 21, Table 22, Table 23, Table 25, Table 26, Table 27, or Table 28, or a combination thereof at multiple time points during treatment, wherein a decrease in expression of the one or more informative genes shown to be expressed, or expressed at increased levels as compared with a control, in individuals having a neurological disorder or at risk for developing a neurological disorder, is indicative that treatment is effective.

The invention also provides a method of assessing the efficacy of a treatment in an individual having a neurological disorder, comprising (i) determining gene expression profile data in a plurality of patient samples, obtained at multiple time points during treatment of the patient, of a selected tissue type; (ii) determining a degree of similarity between (a) the gene expression profile data in the patient samples; and (b) a gene profile produced by a therapy which has been shown to be efficacious in treatment of the neurological disorder; wherein a high degree of similarity is indicative that the treatment is effective.

In one embodiment, the invention also provides a method for assessing the efficacy of a treatment in an individual having at least one autism spectrum disorder comprising (a) determining differential gene expression profile data specific for at least five difference genes set out in Table 3, Table 7, Table 8, Table 9, Table 10, Table 18, Table 19, Table 21, Table 22, Table 23, Table 25, Table 26, Table 27, or Table 28 or a combination thereof, in a plurality of patient samples of a selected tissue type; (b) determining a degree of similarity between (a) the differential gene expression profile data in the patient samples; and (b) a differential gene profile specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, produced by a therapy which has been shown to be efficacious in treatment of the at least one autism spectrum disorder; wherein a high degree of similarity of the differential gene expression profile data is indicative that the treatment is effective.

Another aspect of the invention provides kits. One aspect provides a kit for identifying a compound for treating a behavioral or neurological disorder, comprising (i) a database having information stored therein gene profile data representative of the genetic expression response of selected tissue type samples from subjects or animals that have been subjected to at least one of a plurality of selected behavioral therapies and wherein the tissue has undergone a desired physiological change; and (ii) a computer program for (a) comparing gene expression profile data obtained from assays, where a test compound is administered to a subject or an animal, with the database; and (b) providing information representative of a measure of similarity between the gene expression profile data and one or more stored profiles.

In yet another aspect of the invention, a kit is provided for identifying a compound for treating at least one autism spectrum disorder comprising (a) a database having information stored therein one or more differential gene expression profiles specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, of subjects that have been subjected to at least one of a plurality of selected autism spectrum disorder neurological therapies and wherein the subject has undergone a desired physiological change; and (b) a computer program for comparing gene expression profile data obtained from assays wherein a test compound is administered to a subject with the database and providing information representative of a measure of similarity between the gene expression profile data and one or more stored gene profiles.

In some embodiments of the methods described herein, the test compound comprises an antibody or fragment thereof, a nucleic acid molecule, antisense reagent, a small molecule drug, or a nutritional or herbal supplement. Test compounds can be screened individually, in combination with one or more other compounds, or as a library of compounds. In one embodiment, test compounds include nucleic acids, peptides, polypeptides, peptidomimetics, RNAi constructs, antisense oligonucleotides, ribozymes, antibodies, small molecules, and nutritional or herbal supplements or a combination thereof.

In general, test compounds for modulation of neurological disorders, including those autistic spectrum disorders such as autistic disorder, pervasive developmental disorder-not otherwise specified (PDD-NOS), including atypical autism, Asperger's Disorder, or a combination thereof, can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., Chembridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

V. Methods of Conducting Drug Discovery

Another aspect of the invention provides methods for conducting drug discovery related to the methods and gene chips provided herein.

One aspect of the invention provides a method for conducting drug discovery comprising: (a) generating a database of gene profile data representative of the genetic expression response of at least one selected tissue type (for example, one of the aforementioned neuronal tissue types) from a subject or an animal that was subjected to at least one of a plurality of behavioral therapies and that has undergone a selected physiological change since commencement of the behavioral therapy; (b) selecting at least one gene profile from Table 3, Table 7, Table 8, Table 9, Table 10, Table 18, Table 19, Table 21, Table 22, Table 23, Table 25, Table 26, Table 27, or Table 28, or a combination thereof and selecting at least one target as a function of the selected gene profiles; (c) screening a plurality of small molecule test agents in assays to obtain gene expression profile data associated with administration of the agents and comparing the obtained data with the one or more selected gene profiles; (d) selecting for clinical development test agents that exhibit a desired effect on the target as evidenced by the gene expression profile data; (e) for test agents selected for clinical development, conducting therapeutic profiling of the test compound, or analogs thereof, for efficacy and toxicity in subjects or animals; and (f) selecting at least one test agent that has an acceptable therapeutic and/or toxicity profile.

Another aspect of the invention provides a method for conducting drug discovery comprising: (a) generating a database of gene profile data representative of the genetic expression response of at least one selected neuronal tissue type from a subject or an animal that was subjected to at least one of a plurality of behavioral therapies and that has undergone a selected physiological change since commencement of the behavioral therapy; (b) administering small molecule test agents to test subjects or animals to obtain gene expression profile data associated with administration of the agents and comparing the obtained data with the one or more selected gene profiles; (c) selecting test agents that induce profiles similar to profiles obtainable by administration of behavioral therapy; (d) conducting therapeutic profiling of the selected test compound(s), or analogs thereof, for efficacy and toxicity in subjects or animals; and (e) identifying a pharmaceutical preparation including one or more agents identified in step (e) as having an acceptable therapeutic and/or toxicity profile.

In one embodiment, the database of gene profile data representative of the genetic expression response of at least one selected neuronal tissue type from a subject or an animal that was subjected to at least one of a plurality of behavioral therapies and that has undergone a selected physiological change since commencement of the behavioral therapy comprises at least one gene profile from Table 3, Table 7, Table 8, Table 9, Table 10, Table 18, Table 19, Table 21, Table 22, Table 23, Table 25, Table 26, Table 27, or Table 28, or a combination thereof

### EXAMPLES

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, neurological conditions, cognitive therapies or data analysis methods, all without limitation, can be employed, without departing from the scope of the invention as claimed. The contents of any patents, patent applications, patent publications, or scientific articles referenced anywhere in this application are herein incorporated in their entirety.

### Example 1

### Novel Clustering of Items from the Autism Diagnostic Interview-Revised Identifies Phenotypes that are Associated with Distinct Gene Expression Profiles

This Example demonstrates the use of multiple clustering methods applied to a broad range of ADIR items from a large population (1954 individuals) to identify subgroups of autistic individuals with clinically relevant behavioral phenotypes. Data from large-scale gene expression analyses on lymphoblastoid cell lines derived from individuals who fall within 3 of these subgroups which are reported in the accompanying manuscript show distinct differences in gene expression profiles that in part relate to the severity of the phenotype. Functional and pathway analyses of gene expression profiles associated with the phenotypic subgroups also suggest distinct differences in the biological phenotypes that associate with these subgroups. Based on these analyses, the data suggests that multivariate analysis of the ADIR data using a broad spectrum of the ADIR items and a combination of clustering methods that are typically employed in DNA microarray analyses may be an effective means of reducing the phenotypic heterogeneity of the sample population without restricting the phenotype to only one or a few items which, as pointed out by Lecavalier et al., may associate coincidentally with other variables. Such an approach towards stratification of individuals which utilizes the full spectrum of autism-associated behaviors is expected to aid in the association of genetic and other biological phenotypes with specific forms of ASD.

Methods

Analysis of Data from ADIR Questionnaires to Identify Phenotypic Subgroups

ADIR score sheets were downloaded for 1954 individuals with autism from the Autism Genetic Research Exchange (AGRE) phenotype database. A total of 123 items that were identical or comparable on both 1995 and 2003 versions of the ADIR were included. “Current” and “ever” scores were used for most of these items. Only items scored numerically (0=normal; 3=most severe) were analyzed. A score of 8 for items in the spoken language subgroup indicated that the items were not applicable because of insufficient language and was replaced with a rating of 3. Scores of 8 or 9 for other items (excluding those from the spoken language subgroup), which indicated the item was not asked or not applicable, were replaced with blanks to reflect that no information was available for that item. A score of 1 or 2 on item 19 (LEVELL) indicated an overall language deficit and, as a result, scores for items 20-28 were assigned a score of 3 to reflect impaired language skills, as previously done by Tadevosyan-Leyfer, et al. (2003). Items with scores of 4 in the savant skill subgroup, which meant that the individual possessed an isolated though meaningful skill/knowledge above that of his general functional level or the population norm, were replaced with 3 to maintain consistency of the 0-3 scale across all items. Scores of 7 for some items were changed to a score between 0 and 3 depending on the nature of the question and how it reflected severity with respect to that specific item. A score of −1 indicated missing data (according to AGRE) and was replaced with a blank. Table 1 summarizes the score modifications for each item used for subgrouping of autistic individuals.

Data on ADIR score sheets for 1954 individuals were loaded into MeV (21), a software program created by John Quackenbush and colleagues to analyze microarray gene expression data. Each individual was represented by a horizontal row in the data matrix while ADIR items were represented by vertical columns. Multiple clustering analyses were employed to subgroup individuals on the basis of ADIR item scores and included principal components analysis (PCA), hierarchical clustering (HCL), and k-means clustering (KMC), which is a “supervised” clustering method. A fitness of merit (FOM) analysis was also conducted to estimate the optimal number of clusters, while correspondence analysis (COA) was used to visualize the association of specific items with clusters of individuals. A description of each of these analytical methods is summarized by Saeed et al. (2003).

### Selection of Samples for Large-Scale Gene Expression Analyses

Lymphoblastoid cell lines (LCL) for DNA microarray analyses were selected on the basis of phenotypic clustering of autistic individuals using the methods described above. As described in the results, the application of multiple clustering algorithms to the selected ADIR items from scoresheets of 1954 individuals resulted in 4 reasonably distinct phenotypic subgroups. Samples were selected from 3 of the 4 groups for gene expression analyses. These groups included those with severe language impairment, those with milder symptoms across all domains, and those defined by presence of notable savant skills. Additional selection criteria were applied to exclude all female subjects, individuals with cognitive impairment (Raven's scores <70), those with known genetic or chromosomal abnormalities (e.g., Fragile X, Retts, tuberous sclerosis, chromosome 15q11-q13 duplication), those born prematurely (<35 weeks gestation), and those with diagnosed comorbid psychiatric disorders (e.g., bipolar disorder, obsessive compulsive disorder, severe anxiety). In addition, a score<80 on the Peabody Picture Vocabulary Test (PPVT) was used to confirm language deficits for those in the group identified by cluster analysis as having severe language impairment. In this study, 26-31 cell lines were obtained for each study group, along with 29 cell lines from “control” individuals who were nonautistic siblings of those with autism, matched roughly in age to the individuals with autism.

Cell Culture

The LCL were cultured as previously described according to the protocol specified by the Rutgers University Cell and DNA Repository, which maintains the Autism Genetic Research Exchange (AGRE) collection. Briefly, cells are cultured in RPMI 1640 supplemented with 15% fetal bovine serum, and 1% penicillin/streptomycin. Cultures are split 1:2 every 3-4 days and cells are typically harvested for RNA isolation 3 days after a split while the cultures are in logarithmic growth phase.

Results and Discussion

To reduce the phenotypic heterogeneity of autism for gene expression analyses, several different clustering methods were applied to the scores from ADIR questionnaires (from the AGRE database) describing 1954 autistic individuals. For these analyses, 123 item scores were selected that covered a broad spectrum of behaviors and functions in order to identify phenotypic subgroups of individuals with idiopathic ASD who were characterized by combined symptoms across multiple domains. These domains included language, nonverbal communication, social interactions, play skills, interests and behaviors, physical sensitivities and mannerisms, aggression, and savant skills. The specific items and score adjustments are shown in Table 1.

Principal components analysis of the scores from these individuals shows separation of the autistic individuals into 2 main clusters of undefined phenotype. Hierarchical clustering analysis of the data, however, shows separation of the individuals into multiple clusters, based upon score severity across the different items. A Figure of Merit (FOM) analysis was employed to estimate the optimal number of clusters for supervised clustering analysis. Based on the FOM analysis, K-means analysis was performed, dividing the samples into 4 clusters. This analysis demonstrated that there were easily recognizable distinctions among the groups based upon severity of scores in different domains. For example, one group is characterized by severe language deficits, while another exhibits milder symptoms across the domains. A third group possesses noticeable savant skills while the fourth group exhibited intermediate severity across the domains. Individual samples were color-coded according to KMC grouping in order to observe the distribution of the samples when color was superimposed upon the graph obtained by principal components analysis which shows clear, though not perfect, separation among the groups. It is worth noting that the first 3 components of the PCA capture 38% of the variation among the samples (with 42% represented within the first 4 components). These values are comparable to the 41% sample variation captured across 6 PCA clusters reported by Tadevosyan-Leyfer et al., and suggests that the ADIR items selected in this study are appropriate for identifying phenotypic differences within the autistic population. A correspondence analysis (COA) of the data further suggests that specific clusters of items (e.g., savant skills, aggression, or ritualistic behaviors/resistance to change) are more strongly associated with individuals in certain subgroups than in others (Table 2).

Based upon these combined clustering methods, LCL were selected from individuals represented in 3 of the 4 phenotypic groups for gene expression analyses. These groups included those with severe language impairment, those with a milder phenotype (˜40% of whom had clinical diagnoses of Asperger's Syndrome or PDD-NOS), and those with notable savant skills. Because of the relatively low number of individuals in the “savant” category once other exclusion criteria were applied, a few samples were selected from the group with severe language impairment who also exhibited high scores on savant skills. It should be pointed out that those with savant skills were a minor fraction of the group with severe language impairment. Principal components and K-means analyses of the ADIR item scores for the individuals selected for the microarray studies confirm the separation of the selected samples into 4 phenotypic groups, with the fourth phenotypic group representing individuals with severe language deficits and savant skills.

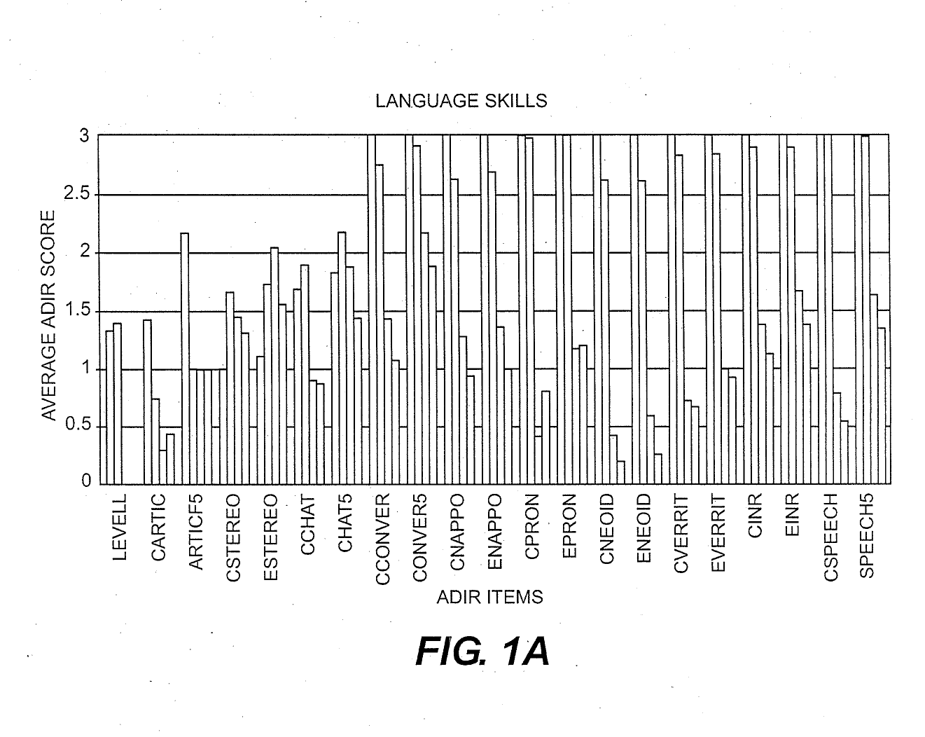

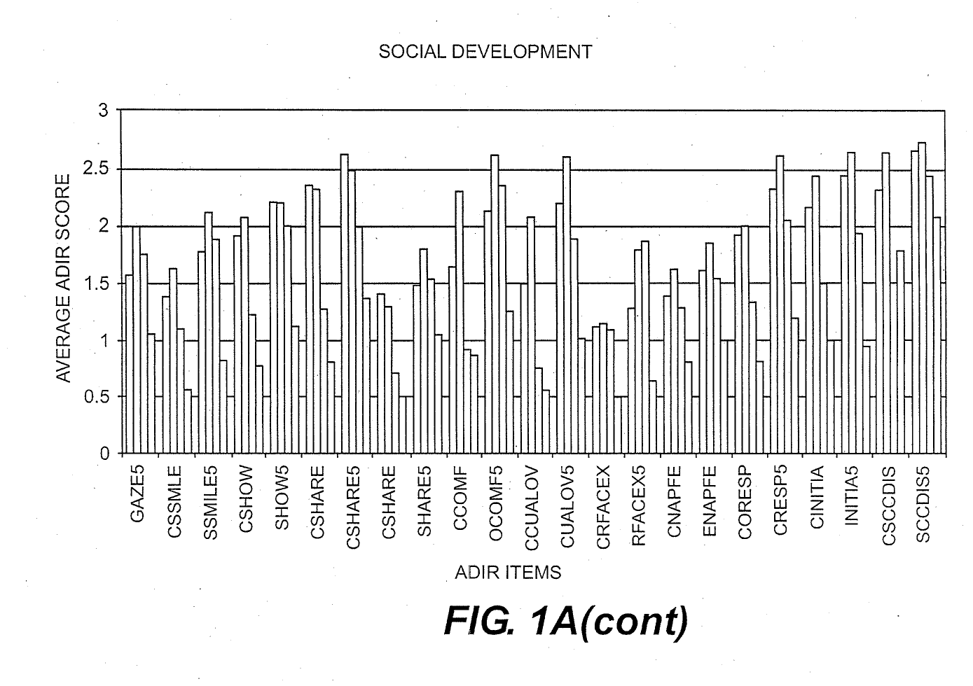

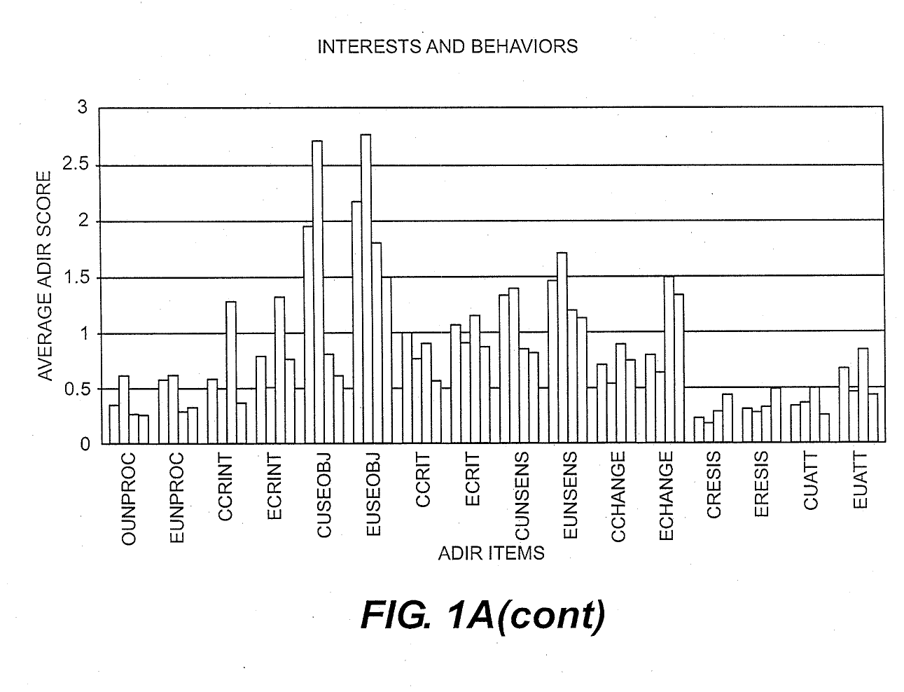

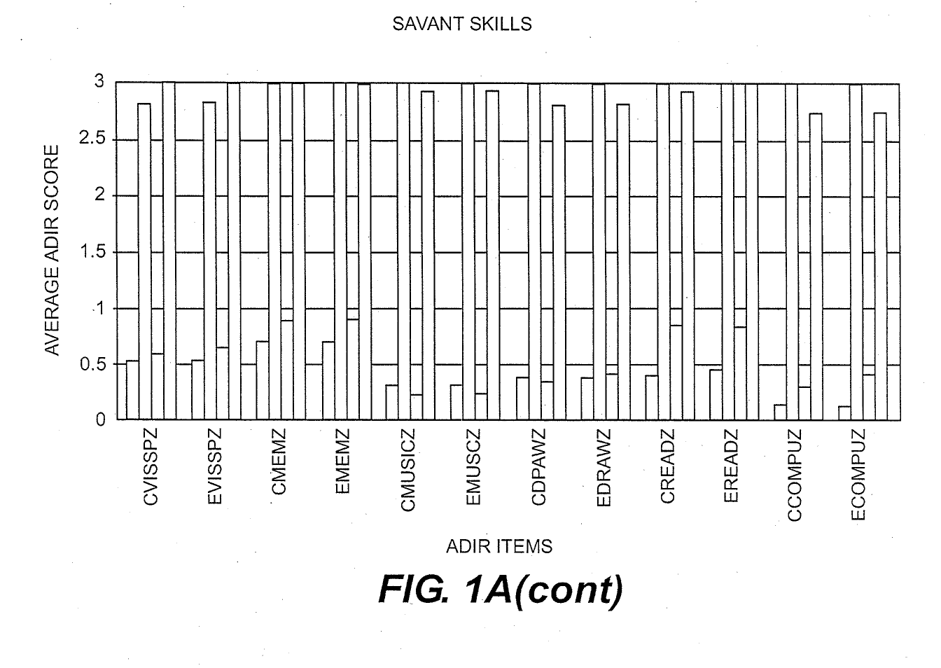

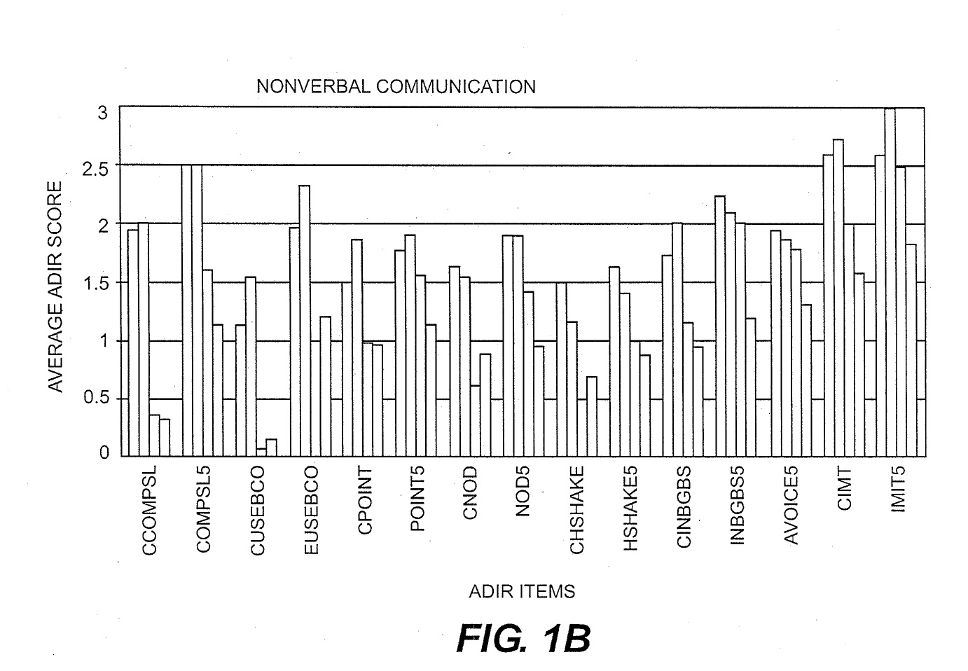

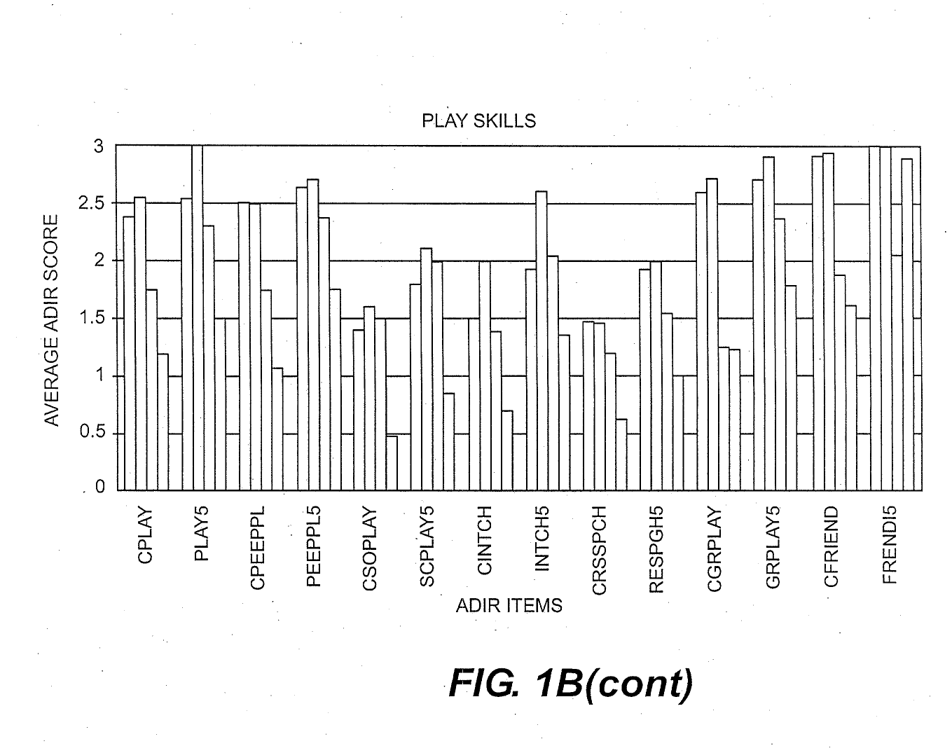

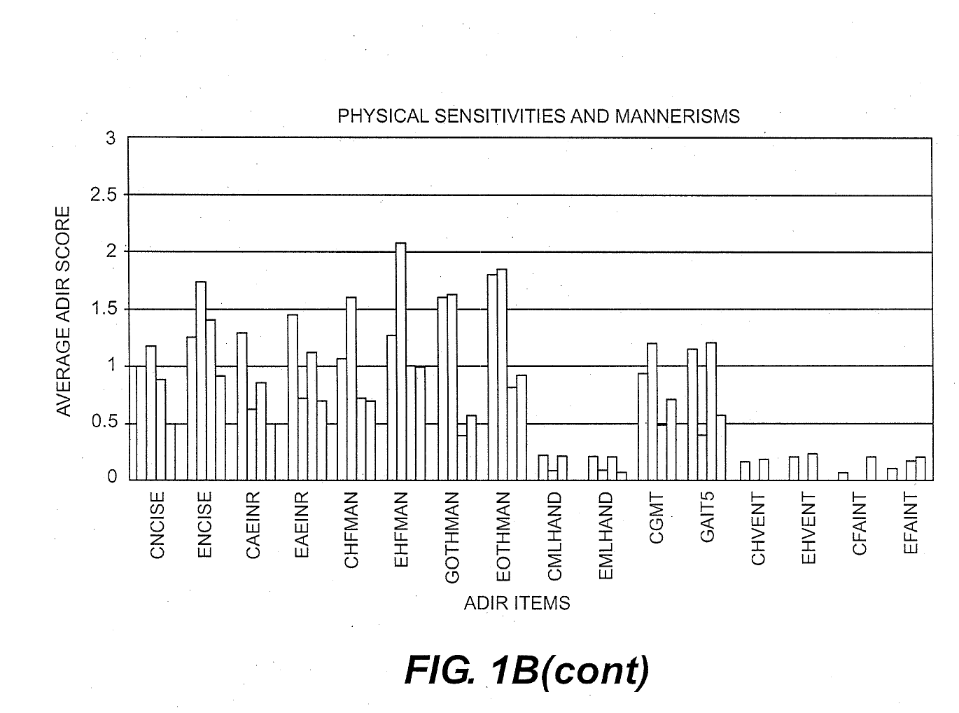

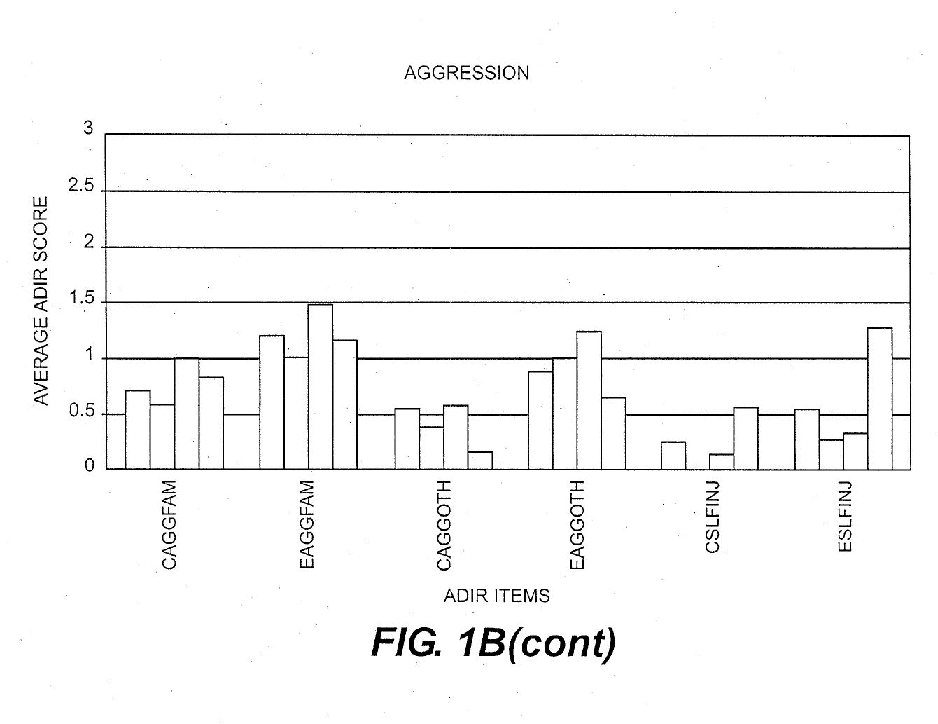

The sum of ADIR scores across all of the items used in this study for the selected individuals, as well as the sum of item scores specific for different functional domains reveals that the group selected for gene expression analysis typically mirrors that of the 1954 individuals from the repository. The profiles for other functional domains (e.g., nonverbal communication, play skills, restricted interests and behaviors) are similar to that representing the sum of all items, for all the individuals in the repository as well as the ones selected for microarray analyses. The average of item scores for each group across the items in each domain as well as the group averages of combined ADIR scores across all items also confirms the phenotypic distinction among the groups. Although there is no significant difference between the average of the sums of the ADIR scores for the mild and savant groups, the ADIR score profiles reveal in FIG. 1 that there are indeed differences among the phenotypic groups across multiple domains of functioning, with the savant group showing lower severity scores than the mild group for almost all items except for savant skills. It is also interesting to note that while individuals in the mild ASD group exhibit lower severity scores in the language domain, most of their scores in the social, nonverbal, and play categories are nearly as severe as those for individuals with severe language impairment, suggesting that higher language abilities do not necessarily correlate well with improved social skills (FIG. 1).

The ADI-R is one of the most widely used diagnostic tests for autism and to many, represents the “gold standard” for identifying individuals with ASD. However, it is only administered after a child presents with abnormal development (e.g., delayed speech) or aberrant behaviors, which typically is noticed between the ages of 2 and 3. Although many studies are currently attempting to identify even earlier signs of abnormal social development (e.g., lack of eye contact, pointing, or shared attention in toddlers, there is still a need to identify definitive molecular markers of ASD that may be used to screen for autism even earlier (pre- or post-natally) as well as to provide targets for therapeutic intervention. A series of studies were embarked upon to identify expressed biomarkers of ASD through the use of large-scale gene expression analyses. Because ADIR scores are the most widely available phenotypic data for the majority of autistic children, the information in this test instrument was used as a starting point to subdivide diagnosed individuals for genomics analyses. EXAMPLE 2 infra demonstrates that subgrouping of autistic individuals by multivariate cluster analysis of ADIR scores which captures the breadth of the disorder within each individual reveals meaningful subgroups or phenotypes of idiopathic autism that can be separated from controls as well as distinguished from each other by gene expression profiling. Detailed bioinformatics analyses of the differentially expressed genes from the resulting subgroups reveal similarities as well as differences in pathways and functions associated with the different phenotypes.

TABLE 1
ADIR items and their score modifications that were employed in this study
ADIR ITEMS AND SCORE MODIFICATIONS
4 -->* 3; 7, 8 & 9 --> blank

| | |
|---|---|
| CVISSP/EVISSP | 106 |
| CMEM/EMEM | 107 |
| CMUSIC/EMUSIC | 108 |
| CDRAW/EDRAW | 109 |
| CREAD/EREAD | 110 |
| CCOMPU/ECOMPU | 111 |
| COMPSL/COMSL5 | 34A |

7 & 8 --> 3; 9 --> blank

| | |
|---|---|
| CPRON/EPRON | 23 |

7 --> 1; 8 --> 3; 9 --> blank

| | |
|---|---|
| CINR/EINR | 26 |
| CUSEOBJ/EUSEOBJ | 72 |

7 --> 2; 9 --> blank

| | |
|---|---|
| CFAINT/EFAINT | 92 |

8 --> 3

| | |
|---|---|
| CUSEBOD/EUSEBOD | 11 |
| LEVELL | 19 |

8 & 9 --> blank

| | |
|---|---|
| CARTIC/ARTIC5 | 14 |
| CSTEREO/ESTEREO | 18 |
| CCHAT/CHAT5 | 16 |
| CPOINT/POINT5 | 30 |
| CNOD/NOD5 | 32 |
| CHSHAKE/HSHAKE5 | 33 |
| CINSGES/INSGES5 | 31 |
| AVOICE5 | 24 |
| CPLAY/PLAY5 | 63 |
| CPEERPL/PEERPL5 | 64 |
| GAZE5 | 42 |
| CSSMILE/SSMILE5 | 43 |
| CSHOW/SHOW5 | 45 |
| COSHARE/OSHARE5 | 46 |
| CSHARE/SHARE5 | 47 |
| COCOMF/OCOMF5 | 49 |
| CQUALOV/QUALOV5 | 51 |
| CRFACEX/RFACEX5 | 52 |
| CQRESP/QRESP5 | 57 |
| CSOPLAY/SOPLAY5 | 65 |
| CINTCH/INTCH5 | 66 |
| CRESPCH/RESPCH5 | 67 |
| CGRPLAY/GRPLAY5 | 68 |
| CSOCDIS/SOCDIS5 | 56 |
| CCIRINT/ECIRNIT | 70 |
| CHFMAN/EHFMAN | 81 |
| CGAIT/GAIT5 | 86 |
| CINITIA/INITIA5 | 61 |
| CIMIT/IMIT5 | 29 |

9 --> blank

| | |
|---|---|
| CUNPROC/EUNPROC | 71 |
| CCRIT/ECRIT | 75 |
| CUNSENS/EUNSENS | 77 |
| CNOISE/ENOISE | 36 |
| CABINAR/EABINR | 78 |
| CCHANGE/ECHANGE | 73 |
| CRESIS/ERESIS | 74 |
| COTHMAN/EOTHMAN | 84 |
| CMLHAND/EMLHAND | 82 |
| CAGGFAM/EAGGFAM | 91B |
| CAGGOTH/EAGGOTH | 91C |
| CSLFINJ/ESLFINJ | 90 |
| CHVENT/EHVENT | 80 |

8 --> 3; 9 --> blank

| | |
|---|---|
| CCONVER/CONVER5 | 20 |
| CINAPPQ/EINAPPQ | 22 |
| CNEOID/ENEOID | 24 |
| CVERRIT/EVERRIT | 25 |
| CSPEECH/SPEECH5 | 28 |
| CINAPFE/INAPFE5 | 53 |
| CFRIEND/FREND15 | 69 |

7 --> 1; 6 --> 3; 9 --> blank

| | |
|---|---|
| CUATT/EUATT | 76 |

all −1 become blank
NOTE:
if item 19 has a score of 1 or 2, items 20-28 are scored as 3
* --> “score converted to”

TABLE 2
Clusters of associated items identified by correspondence
analysis (COA) of the ADIR data for 1954 individuals from the
AGRE repository
COA clusters from 1954 individuals

| 1 | | 3 | | |
|---|---|---|---|---|
| (turquoise) | 2 (lime) | (lavender) | 4 (pink) | |
| CVISSPZ | GAIT5 | CSTEREO | LEVELL | OSHARE5 |
| EVISSPZ | CAGGFAM | ESTEREO | CCOMPSL | CSHARE |
| CMEMZ | EAGGFAM | CUNPROC | COMPSL5 | SHARE5 |
| EMEMZ | CAGGOTH | EUNPROC | CUSEBOD | COCOMF |
| CMUSICZ | EAGGOTH | CCIRINT | EUSEBOD | OCOMF5 |
| EMUSICZ | CSLFINJ | ECIRINT | CARTIC | CQUALOV |
| CDRAWZ | ESLFINJ | CCRIT | ARTICF5 | QUALOV5 |
| EDRAWZ | CHVENT | ECRIT | CCHAT | CRFACEX |
| CREADZ | EHVENT | CNOISE | CHAT5 | RFACEX5 |
| EREADZ | CFAINT | ENOISE | CCONVER | CINAPFE |
| CCOMPUZ | EFAINT | CABINR | CONVER5 | EINAPFE |
| ECOMPUZ | | EABINR | CINAPPQ | CQRESP |
| | | CCHANGE | EINAPPQ | QRESP5 |
| | | ECHANGE | CPRON | CINITIA |
| | | CRESIS | EPRON | INITIA5 |
| | | ERESIS | CNEOID | CSOPLAY |
| | | CUATT | ENEOID | SOPLAY5 |
| | | EUATT | CVERRIT | CINTCH |
| | | CMLHAND | EVERRIT | INTCH5 |
| | | EMLHAND | CINR | CRESPCH |
| | | | EINR | RESPCH5 |
| | | | CSPEECH | CGRPLAY |
| | | | SPEECH5 | GRPLAY5 |
| | | | CPOINT | CFRIEND |
| | | | POINT5 | FREND15 |
| | | | CNOD | CSOCDIS |
| | | | NOD5 | SOCDIS5 |
| | | | CHSHAKE | CUSEOBJ |
| | | | HSHAKE5 | EUSEOBJ |
| | | | CINSGES | CUNSENS |
| | | | INSGES5 | EUNSENS |
| | | | AVOICE5 | CHFMAN |
| | | | CIMIT | EHFMAN |
| | | | IMIT5 | COTHMAN |
| | | | CPLAY | EOTHMAN |
| | | | PLAY5 | CGAIT |
| | | | CPEERPL | |
| | | | PEERPL5 | |
| | | | GAZE5 | |
| | | | CSSMILE | |
| | | | SSMILE5 | |
| | | | CSHOW | |
| | | | SHOW5 | |
| | | | COSHARE | |

### Example 2

### Gene Expression Profiling Differentiates Autism Case-Controls and Phenotypes of ASD: Evidence for Circadian Rhythm Dysfunction in Severe Autism

As described in EXAMPLE 1 supra, several clustering algorithms were applied to data from the Autism Diagnostic Interview-Revised (ADIR) questionnaires in an attempt to divide nearly 2000 autistic individuals into phenotypic subgroups based upon severity across 123 ADIR items. This approach differs significantly from that employed by other investigators in that the subgroups are defined by multiple items within different behavioral or functional categories, including spoken language, nonverbal communication, social skills, play skills, physical attributes and sensitivities, aggression, and savant skills, while many other studies utilize at most several item scores within a single category to define subgroups of individuals. Another aspect of the approach that differs from previous analyses is that the method employs multiple clustering algorithms to the data which results in a clearer and more intuitive phenotypic description of the subgroups. Using these combined methods to identify both severe and mild subgroups of ASD individuals as well as those with notable savant skills, it is now demonstrated that discrimination of autistic from nonautistic individuals based upon gene expression profiles. In addition, both qualitative and quantitative differences in gene expression are observed between the subgroups. Furthermore, several phenotypes of autism can also be distinguished by pathway analyses, corroborating the distinct biological phenotypes of ASD.

### Materials and Methods

### Cell Culture

The LCL were cultured as previously described (Hu V W, Frank B C, Heine S, Lee N H & Quackenbush J (2006)) according to the protocol specified by the Rutgers University Cell and DNA Repository, which maintains the Autism Genetic Research Exchange (AGRE) collection of biological materials from autistic individuals and relatives. Briefly, cells are cultured in RPMI 1640 supplemented with 15% fetal bovine serum, and 1% penicillin/streptomycin. Cultures are split 1:2 every 3-4 days and cells are typically harvested for RNA isolation 3 days after a split while the cultures are in logarithmic growth phase.

### Gene Expression Analyses on Spotted DNA Microarrays

Gene expression profiling is accomplished using TIGR 40K human arrays as previously described (Hu V W, Frank B C, Heine S, Lee N H & Quackenbush J (2006)). Total RNA was isolated from LCL using the TRIzol (Invitrogen) isolation method according to the manufacturer's protocols, and cDNA was synthesized, labeled, and hybridized to the microarrays as described in our earlier study, with the exception that cDNA from each sample was labeled with Cy-3 dye and hybridized against Cy-5 labeled reference cDNA prepared from Universal human RNA (Stratagene). This “reference” design allows the flexibility to perform different comparisons among the samples since all expression values are against a common reference. After hybridization, washing of the arrays, and laser scanning to elicit dye intensities for each element on the array, the intensity data was normalized and filtered using Midas and analyzed using MeV, which are open-access software programs for DNA microarray analyses (Saeed A I, et al (2003)). All analyses were performed with a 70% data filter which means that each gene included in the analyses must have an expression value in 70% of the samples. Significant differentially expressed genes were identified using the Significance Analysis of Microarrays (SAM) (Tusher V G, Tibshirani R & Chu G (2001), Chu T-, Weir B & Wolfinger R (2002)) module within MeV for both 2-class and 4-class analyses.

### Quantitative PCR Analysis

Select genes were confirmed by real time quantitative RT-PCR (qRT-PCR) on an ABI Prism 7300 Sequence Detection System using Invitrogen's Platinum SYBR Green qPCR SuperMix-UDG with ROX. Total RNA (same preparations used in microarray analyses) was reverse transcribed into cDNA using the iScript cDNA Synthesis Kit (Bio-Rad, Hercules, Calif.). Briefly, 1 μg of total RNA was added to a 20 μl reaction mix containing reaction buffer, magnesium chloride, dNTPs, an optimized blend of random primers and oligo(dT), an RNase inhibitor and a MMLV RNase H+ reverse transcriptase. The reaction was incubated at 25° C. for 5 minutes followed by 42° C. for 30 minutes and ending with 85° C. for 5 minutes. The cDNA reactions were then diluted to a volume of 50 μl with water and used as a template for quantitative PCR.

Quantitative RT-PCR primers for genes identified by microarray analysis as differentially expressed were selected for specificity by the National Center for Biotechnology Information Basic Local Alignment Search Tool (NCBI BLAST) of the human genome, and amplicon specificity was verified by first-derivative melting curve analysis with the use of software provided by PerkinElmer (Emeryville, Calif.) and Applied Biosystems. Sequences of primers used for the real-time RT-PCR are given in Table 11.

Quantitative RT-PCR analyses were performed on all samples, with quantification and normalization of relative gene expression using the comparative threshold cycle method as described previously (Hu V W, Frank B C, Heine S, Lee N H & Quackenbush J (2006)). The expression of the “housekeeping” genes MDH1 (NM_{—}005917), ARF1 (NM_{—}001024227) and ACSL5 (NM_{—}016234) were used for normalization as these genes did not exhibit differential expression in our microarray assays. The qRT-PCR reactions were done in triplicate.

### Gene Ontology and Pathway Analyses

The datasets of differentially expressed genes between autistic probands and unrelated controls were analyzed using Ingenuity Pathway Analysis (IPA) and Pathway Studio 5 to identify relational gene networks, high level functions, and small molecules associated with the gene regulatory networks. Gene ontology analyses were also performed on the datasets using DAVID Bioinformatics Resources (david.abcc.ncifcrf.gov) for additional functional annotation (G. D, Jr., et al (2003)).

### Results

In EXAMPLE 1 supra, a novel clustering method is provided for stratifying autistic individuals according to phenotypes which encompass 123 scores on 63 distinct items on the Autism Diagnostic Interview-Revised (ADIR) questionnaire, most of which are represented by 2 separate scores related to “current” (existing) or “ever” (previously exhibited) behaviors. In this EXAMPLE, the gene expression profiles of 3 of the 4 phenotypic subgroups that resulted from the cluster analyses of ADIR scores were analyzed and demonstrate different functions overrepresented within the different subgroups that are suggestive of distinct “biological phenotypes”. To test proof-of-principle that the phenotypic subgroups can be differentiated from each other by gene expression profiles, the group with severe language impairment and high severity scores across most of the ADIR items used for clustering (except savant skills), the mild group comprised of individuals many of whom were clinically diagnosed with PDD-NOS or Asperger's Syndrome who exhibited distinctly lower severity ADIR item scores, and the individuals with noticeably high scores in the savant skills categories to identify genes that may be associated with this unusual and interesting trait, were analyzed in this Example. The intermediate group was not included in this study because it was important to be able to first demonstrate differences between groups at the extreme ends of the spectrum.

### DNA Microarray Analyses of ASD Phenotypic Subgroups Show Quantitative and Qualitative Differences in Gene Expression

Gene expression profiles of lymphoblastoid cell lines (LCL) from each of the autistic individuals studied and age-matched controls were obtained by cDNA microarray analyses. A 2-class analysis of the data reveals a set of significant differentially expressed genes (FDR≦0.05) that distinguish controls from all autistic samples (Table 7). Interestingly, when the samples from the autistic individuals are grouped according to phenotype, the gene expression matrix from this analysis shows a gradient in differential gene expression for some genes in which the level of gene expression reflects the overall severity of the ASD phenotype relative to controls. Separation of the 3 ASD phenotypes from each other as well as from controls was further revealed by a 4-class SAM analysis of the microarray data (FDR≦0.0001) from all individuals. To reduce the dimensionality of the data, principal components analysis (PCA) was applied to significant genes derived from the 4-class SAM analysis. This nonsupervised cluster analysis also demonstrated that the phenotypic subgroups can be differentiated from each other as well as from controls, although there is still some mixing of the phenotypes, particularly between the “savant” group and controls. However, it should be noted that 8 of the controls are siblings of those with “savant” skills and that it is known that genotype plays a large role in overall gene expression profiles. Pavlidis template matching (PTM) of significant genes from the 4-class analysis to identify genes that differentiate all autistic subjects from the nonautistic controls further illustrated the quantitative relationship between gene expression and the severity of ASD as defined by ADIR cluster analyses. These analyses clearly show qualitative as well as quantitative differences in gene expression profiles that relate to “phenotype” and emphasize the need to identify and utilize more homogeneous samples for biological analyses.

Towards this goal, each ASD group was treated as a separate class and performed 2-class statistical analyses on the gene expression data obtained from each of the groups in comparison to nonautistic controls to identify the differentially expressed genes that were specific to each group. The gene expression profiles of genes that were differentially expressed between each of the ASD subgroups and controls, as well as PCA plots demonstrating separation of individuals from each of the subgroups from controls on the basis of gene expression profile reveal that the first 3 principal components of the respective PCA analyses for language (L), mild (M), and savant (S) subgroups represent 56.7%, 38.2%, and 30.2% of the variability reflected in the gene expression data in comparison to only ˜25% of the variability when all autistic samples are treated as one group. Lists of the differentially expressed genes for these 3 ASD subtypes are provided respectively in Tables 8-10, wherein Table 8 is a subset of the ˜4000 differentially expressed genes for the L subgroup with a false discovery rate (FDR) of 5%. Table 27 contains the most differentially expressed genes from this dataset, with an absolute log 2 expression ratio 0.3.

Overlapping as Well as Unique Genes are Associated with Each ASD Subgroup

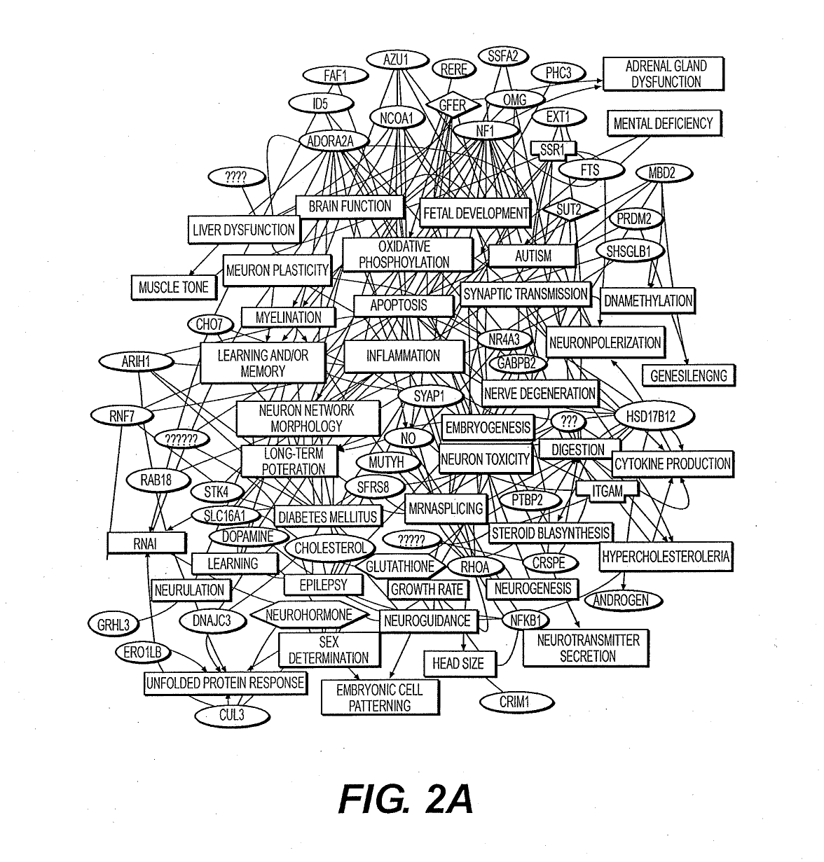

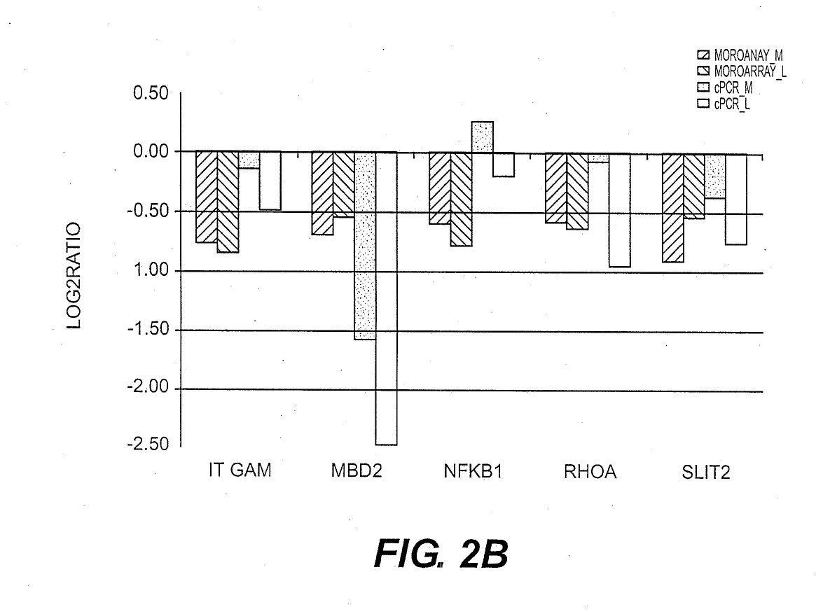

Venn diagram analysis reveals that there are five (5) overlapping significantly differentially expressed transcripts among the 3 ASD groups. Pathway analysis of the overlapping genes between the L and M subgroups reveals a network of genes that affect common functional targets, such as synaptic transmission and plasticity, neurogenesis, neuron guidance, learning and memory, and myelination that have been identified as dysfunctional in ASD (FIG. 2A). Of additional interest are the disorders associated with this set of genes, including autism, mental deficiency, epilepsy, head size (macrocephaly), muscle tone (hypotonia), and hypercholesterolemia, which have been reported in subsets of individuals with ASD. Key regulators of the functional targets were confirmed by quantitative reverse transcriptase-polymerase chain reaction RT-PCR (qRT-PCR) (FIG. 2B). Table 3 lists the 5 overlapping significantly differentially expressed transcripts across all 3 ASD subgroups. What is intriguing about this set is that all 5 transcripts are novel and uncharacterized genes which are associated with cellular response to androgens as revealed by gene expression studies on Androgen Insensitivity Syndrome and androgen-sensitive and androgen-insensitive prostate tumors (Holterhus P M, Hiort O, Demeter J, Brown P O & Brooks J D (2003), Zhao H, et al (2005)). At least 3 of these genes have been shown to be downregulated in LCL in response to dihydrotestosterone.

### Functional Analyses of the Different Subgroups of ASD on the Basis of Gene Expression Profiles: Evidence for Distinct Biological Phenotypes

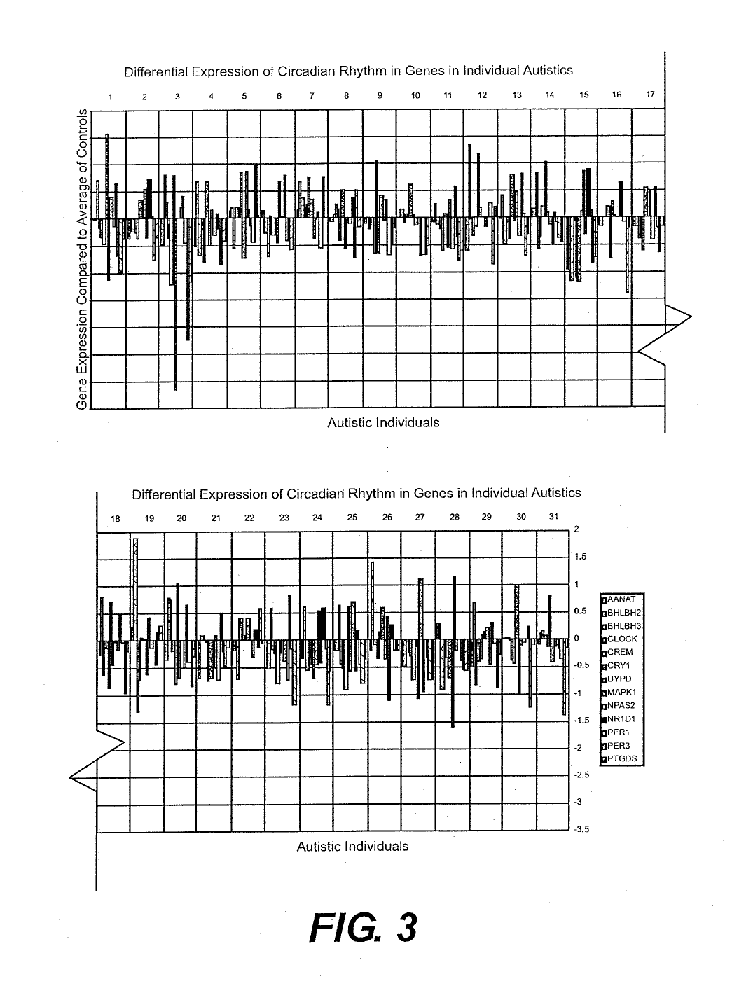

To understand the differences in the pathways and functions that are affected in each of the phenotypic groups, pathway and functional analyses were conducted on each of the gene datasets (in Tables 8-10) derived from comparison of the respective phenotypes versus controls. Table 4 summarizes the results obtained from IPA for each group in terms of categories in molecular and cellular functions, canonical pathways, and toxicity that are significantly enriched with differentially expressed genes. It is clear from this summary that biological functions and pathways are most altered in the severely language-impaired group and the least altered in the “savant” group. Among the genes relating to molecular and cellular functions, cell death genes are overwhelmingly represented in the group with severe language deficits, while genes involved in cell growth and proliferation and cellular movement are differentially expressed in both the language and mild phenotypes, albeit to a greater extent in the group with severe language deficits. Among the genes involved in specific canonical pathways are those related to liver toxicity (hepatic stellate cell activation, fibrosis, and cholestasis) which are overrepresented in the severely language-impaired group, but not in the mild group. It is proposed that the dysregulation of at least some of these genes may be responsible for gastrointestinal disorders that are often associated with autism. Further comparison of the severe and mild groups on the basis of genes that are enriched for neurological functions and disorders revealed not only differences in the number of genes associated with cell death in the severely language-impaired group, but also a greater number of differentially expressed genes involved with various neurological disorders commonly associated with autism, such as allodynia, catalepsy, hypernocieption, and epilepsy (Table 5). Particularly noteworthy are the 13 genes that are involved in the regulation of circadian rhythm which also affect many of the neurological functions and disorders commonly associated with ASD, such as synaptic plasticity, learning, memory, inflammation, cytokine production, digestion. All 13 of the genes in this network (AANAT, BHLHB2, BHLHB3, CLOCK, CREM, CRY1, DPYD, MAPK1, NPAS2, NR1D1, PER1, PER3, and PTGDS) are differentially expressed to different extents only in individuals in the severely language-impaired (L) group (FIG. 3) and 6 have been confirmed by qRT-PCR (Table 6). An additional 2 circadian rhythm genes, NFIL3 and RORA, are found in the expanded dataset for this group (Table 27).

Many Differentially Expressed Genes are Associated with Autism QTL Identified by Genetic Analyses

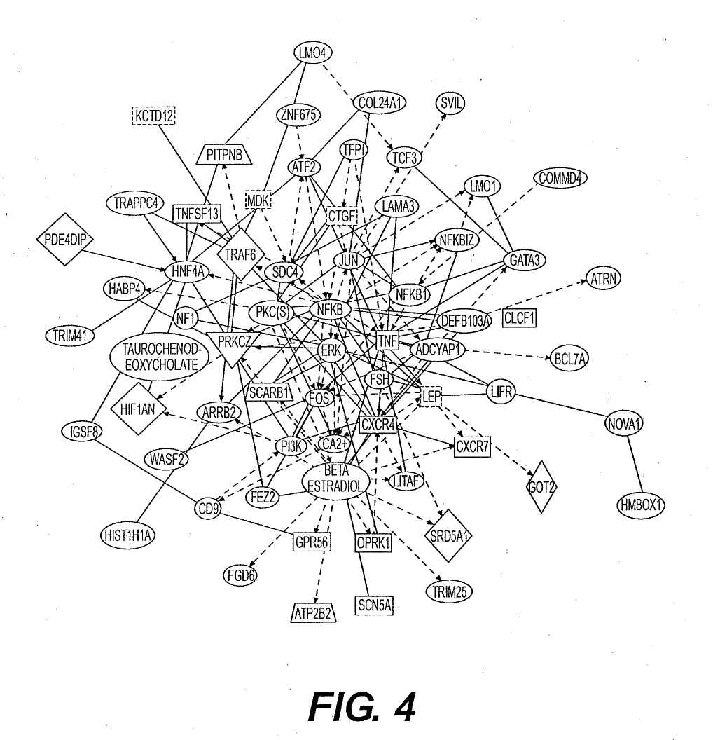

Gene expression analyses indicate that there are hundreds to thousands of genes that are differentially expressed between LCL of nonautistic individuals and those of each of the 3 ASD groups studied. To investigate whether these genes bear any relationship to genetically identified autism susceptibility loci, the differentially expressed genes to quantitative trait loci (QTL) reported by seven laboratories were mapped (Alarcon M, Yonan A L, Gilliam T C, Cantor R M & Geschwind D H (2005), Chen G K, Kono N, Geschwind D H & Cantor R M (2006), Duvall J A, et al (2007), Philippe A, et al (1999), Szatmari P, et al (2007), Bailey A, et al (1998), Weiss L A, et al (2008)). On average, about 27-33% of the differentially expressed genes are associated with autism QTL, across all subgroups and the autistic samples combined (FIG. 4). There is significant enrichment of differentially expressed genes in QTLs on chromosomes 2, 4, 7, 10, 16, 17, and 19 for the language subgroup, as indicated in the figure, as well as on chromosomes 7, 16, and 17 in the mild and combined autistic groups, the latter of which also shows enrichment on chromosome 10. It is notable that all of these chromosomes have undergone intensive genetic analyses as “hot spots” with respect to autism. Thus, the layering of gene expression data onto genetic data may be a useful means of prioritizing candidate genes for further functional and genetic analyses.

### Discussion

Genetic and other biological analyses of idiopathic autism which makes up at least 70-80% of ASD cases have been hampered by the inherent heterogeneity of presentation of ASD in different individuals which, in turn, increases the noise in the experimental data. The phenotypic heterogeneity of clinical samples obtained through the AGRE/NIMH tissue repository was reduced by subgrouping/stratifying individuals based upon cluster analyses of 123 scores on 63 items from their respective ADIR scoresheets, some of which are queried with respect to current behaviors and previously exhibited behaviors. While other studies have utilized several ADIR item scores within a specific domain (e.g., spoken language, nonverbal communication, social skills or repetitive behaviors) to stratify ASD individuals for genetic analyses, this is the first study to subgroup individuals on the basis of ADIR item scores that reflect the full range of deficits commonly associated with ASD. It is demonstrated herein that the gene expression profiles associated with each of the 3 ASD phenotypes that were selected for DNA microarray analyses show both qualitative and quantitative differences which are dependent on ASD phenotype. Also demonstrated is the overlap of some of the differentially expressed genes among subgroups which indicates common underlying biological deficits in ASD as well as differences that suggest dysregulation of specific pathways in a particular subgroup of ASD.

### ASD Phenotypic Subgroups can be Distinguished on the Basis of Gene Expression Profiling

The gene expression profiles associated with each of the 3 ASD phenotypes that were selected for DNA microarray analyses show both quantitative and qualitative differences which are dependent on ASD phenotype. The quantitative differences that were revealed in a 2-class analysis of the gene expression profiles of all autistic probands vs. controls were particularly surprising and likely identify genes that influence the severity of ASD. These genes would thus serve as good candidates for expression quantitative trait loci QTL (eQTL) analyses which, in turn, will help to prioritize genes for in-depth genetic association and linkage studies. It should be pointed out that the gradient of gene expression is only apparent when the samples are clustered according to ASD subtype, thus validating the value of our clustering methods which were applied to selected ADIR item scores. Genes whose expression levels are qualitatively, but not quantitatively, dependent on subgroups (data not shown) also present a strong case for subtyping ASD individuals according to our methods since averaging gene expression values across all samples would dampen the overall expression differences from controls and obscure the biological differences between the subgroups. It is therefore suggested that such clustering of individuals to reduce the phenotypic heterogeneity of the study groups will also be of value to genetic and other biological analyses of ASD.

### Overlapping Differentially Expressed Genes May Underlie Basic Deficits in ASD

Venn diagram analysis of the number of overlapping differentially expressed genes among the 3 ASD groups revealed that the largest overlap occurred between the severe (L) and mild (M) groups. Among the major functions associated with this set of overlapping genes are apoptosis and inflammation, as well as many neurological and metabolic processes commonly associated with ASD, such as myelination, neuron plasticity, synaptic transmission, and hypercholesterolemia (FIG. 2). Genes which were confirmed by qRT-PCR analyses (ITGAM (integrin, alpha M (aka CD11b)), NFKB1 (nuclear factor of kappa light polypeptide gene enhancer in B-cells 1), RHOA (ras homolog gene family, member A), SLIT2 (slit homolog 2), and MBD2 (methyl-CpG binding domain protein 2) are all strong candidates for further evaluation of their role in ASD. ITGAM is involved in synapse formation and neuron toxicity, and is associated with chronic neural inflammation and microglial activation. Similarly, the transcription factor NFKB1 is also a key regulator of inflammatory responses which have been associated with ASD (Zimmerman A W, et al (2005) Jyonouchi H, Sun S & Le H (2001), DeFelice M L, et al (2003)). RHOA and SLIT2 are components of the synpatogenesis/axon guidance pathway which is strongly implicated in ASD (Persico A M & Bourgeron T (2006), Jamain S, et al (2003), Szatmari P, et al (2007), Matzke A, et al (2007)). These biological processes (inflammation, axon guidance) as well as others shown in FIG. 2 (e.g., apoptosis, myelination, steroid biosynthesis, and sex determination) replicate those identified in our previous gene expression studies of monozygotic twins discordant in diagnosis or severity of autism ((Hu V W, Frank B C, Heine S, Lee N H & Quackenbush J (2006)) and autistic-nonautistic sib pairs (See Example 3, infra). Altered expression of MBD2, a methyl-CpG binding protein, suggests the role of epigenetic factors in ASD. Indeed, several mutations have been identified in this family of transcriptional regulator proteins in autistic patients (Li H, Yamagata T, Mori M, Yasuhara A & Momoi M Y (2005)) and MECP2, in particular, is responsible for Rett's Syndrome, a genetically defined ASD. The previous observation of gene expression differences between monozygotic twins discordant in diagnosis or severity of autism further supports the role of epigenetic regulation in ASD ((Hu V W, Frank B C, Heine S, Lee N H & Quackenbush J (2006)).

The most intriguing of the overlapping genes are the 5 novel genes that are shared by all three ASD groups because of their potential importance to core symptoms of ASD (Table 4). As mentioned earlier, all 5 of these highly significant differentially expressed genes have been observed to be differentially regulated within the context of androgen insensitivity (Holterhus P M, Hiort O, Demeter J, Brown P O & Brooks J D (2003), Zhao H, et al (2005)). This, in itself, is very interesting because of the hypothesis that higher levels of fetal testosterone may be a risk factor for ASD (Baron-Cohen S, Knickmeyer R C & Belmonte M K (2005), Knickmeyer R, Baron-Cohen S, Raggatt P & Taylor K (2005), Knickmeyer R C & Baron-Cohen S (2006)). In fact, there is experimental support for this hypothesis, both from analysis of serum levels of androgens in individuals with ASD (Ingudomnukul E, Baron-Cohen S, Wheelwright S & Knickmeyer R (2007), Geier D A & Geier M R (2006)), as well as from our own studies (manuscript submitted) which show dysregulation of genes within the steroid hormone biosynthetic pathway in LCL from ASD probands as well as higher testosterone levels in their LCL extracts relative to their respective nearly age-matched siblings. Clearly, more research is needed to identify and characterize these novel genes as well as to demonstrate their function within the context of ASD.

Subgroup-Specific Genes Suggest Dysregulation of Specific Pathways Associated with the Respective ASD Phenotypes

Subtyping of ASD individuals prior to gene expression analyses also revealed differentially expressed genes that were unique to each subgroup. Thirteen circadian rhythm regulatory or responsive genes were among the genes identified as differentially expressed in the most severe (L) subgroup but not in the mild or savant groups, suggesting a connection between dysregulation of circadian rhythm and the severity of this phenotype. In 2002, Wimpory et al. proposed a relationship between social timing, “clock” (circadian rhythm) genes, and autism, and more recently demonstrated association of PER1 and neuronal PAS domain protein 2 (NPAS2), but not other circadian rhythm genes, with autistic disorder (Nicholas B, et al (2007)). This very interesting hypothesis is based in part upon the prevalence of sleep disorders in ASD which suggest deficits in the regulation of circadian rhythm (Malow B A (2004), Johnson K P & Malow B A (2008)). A recent report that Fragile X-related proteins regulate transcriptional activity of the clock genes provides additional experimental support for the involvement of circadian rhythm in ASD (Zhang J, et al (2008)). Bourgeron has further proposed a connection between clock and synaptic genes (NLGN3, NLGN4, NRXN1, and SHANK3) in autism spectrum disorders (Bourgeron T (2007)). He also pointed out the importance of gene dosage in the balance of excitatory and inhibitory signaling at the synapse and suggested the possible importance of the circadian rhythm in controlling such signaling and hence the severity of ASD. The significance of gene dosage effects (which can be manifested by altered gene expression) as contributors to ASD are emphasized by recent studies which show that copy number variants can be associated with both familial and spontaneous forms of ASD. Our network analysis of the 13 circadian rhythm genes that are differentially expressed only in the severe ASD group shows the relationships between these genes and many neurological functions as well as disorders typically observed in ASD. It should be mentioned that multiple genes (though not all 13) are differentially expressed in each individual (FIG. 3), suggesting a multi-hit mechanism of dysregulation of the circadian rhythm in the most severe phenotype of ASD.

Among the genes confirmed by qRT-PCR are arylalkylamine N-acetyltransferase (AANAT), basic helix-loop-helix domain containing, class B, 2 (BHLBH2), CRY1 (cryptochrome 1), neuronal PAS domain protein 2 (NPAS2), Period 3 (PER3), and dihydropyrimidine dehydrogenase (DPYD). A significant decrease is observed for AANAT, an enzyme which catalyzes the rate-limiting first step of the biochemical conversion of serotonin to melatonin, a key regulator hormone of the circadian cycle. A reduction in this enzyme would be consistent with the abnormally low levels of melatonin which have been reported in a number of studies of autistic patients. Overexpression of BHLHB2/DEC1, which regulates the expression of the master circadian regulator genes CLOCK and BMAL1, has also been shown to delay the phase of several clock genes (e.g., DEC1, DEC2, and PER1) which contain E boxes in their regulatory regions. CRY1 and PER3 are also transcriptional modulators of CLOCK/BMAL1 while NPAS2 is a CLOCK analog expressed primarily in brain tissues. While not directly involved in the control of circadian rhythm, DPYD is a major target of the clock genes and a particularly important gene with respect to neurological functions. In fact, DPYD deficiency leads most frequently to epilepsy, mental and motor retardation (all symptoms associated with subgroups of autism), and other developmental disorders, with 18% of DPYD-deficient individuals receiving a diagnosis of autism. Metabolically, DPYD catalyzes the breakdown of uracil to β-alanine, which activates both GABA_{A} and glycine receptors with the same efficacy as their respective natural ligands. Thus, a deficiency in DPYD or the resultant subnormal levels of β-alanine can be predicted to lead to decreased inhibitory signaling activity at the synapse. Interestingly, anti-convulsant medications which are often prescribed as a therapeutic regimen for epilepsy associated with DPYD deficiency are also efficacious in improving behaviors in a subgroup of ASD individuals, even without apparent seizures. It is therefore suggested that evaluation of DPYD status, β-alanine levels, or circadian rhythm function in ASD individuals might be helpful in identifying those patients that would most benefit from this type of medication. Overall, the net effect of the observed changes in gene expression is the dysregulation of circadian rhythm in this most severely affected subgroup of ASD individuals. Since the circadian rhythm affects not only neurological but also endocrine, gastrointestinal, and cardiovascular functions, dysregulation of these genes can also have a systemic impact on affected individuals, causing many of the symptoms that are often associated with ASD. Thus, it may be proposed that interventions aimed at normalizing the circadian “clock” may ameliorate some of the symptoms associated with ASD for this subgroup.

### Summary

This Example demonstrates the value of subdividing individuals with ASD on the basis of cluster analyses of ADIR scores that incorporate all 3 core domains of ASD as described in the accompanying manuscript. Stratifying the sample by cluster analyses revealed quantitative differences in gene expression that appear to correlate with severity of ASD phenotype as well as gene expression profiles for each subtype that associate a “biological phenotype” (i.e., gene expression) to the respective functional/behavioral phenotype. The biological phenotypes reveal differences in some of the biological functions affecting individuals with ASD, such as circadian rhythm dysregulation in the severe (L) phenotype, suggesting possible therapeutic interventions specific to this subgroup. On the other hand, overlapping genes among the phenotypes indicate dysregulation of genes controlling both neurological and metabolic functions that may lie at the core of ASD. Of particular interest for future studies are the 5 novel genes that are significantly differentially expressed across all 3 subgroups of ASD identified here. Because of their apparent sensitivity to androgens based upon gene expression data deposited into the Gene Expression Omnibus (GEO) repository for data from late-scale gene expression analyses (as well as our unpublished data), these genes may underlie the prominent 4:1 male-to-female sex bias in susceptibility to ASD.

In summary, this Example demonstrates that:
1) The level of expression of some genes relates directly to the severity of the phenotype, and may serve as useful candidates for eQTL analyses;
2) Network analysis of genes that are shared between the severely language-impaired and mild ASD groups reveal a set of genes that are probably critical with respect to the neurological and metabolic abnormalities of ASD;
3) Differences between affected functions and pathways among the different phenotypic groups may be responsible for the differences in symptom severity observed in autism.
   Finally, the results suggest that some of the neurological manifestations of ASD are at least in part the result of dysregulated signaling and metabolic pathways that are reflective of a systemic disorder which, once identified, may be treatable. The implications of these findings as well as those of others who have identified gene signatures of psychiatric disorders in lymphoblasts support the use of non-neuronal tissues, including patient-derived LCL and primary peripheral cells, to investigate the pathobiology of ASD.

TABLE 3
Five overlapping differentially expressed transcripts across all 3 ASD
subgroups analyzed.

| | | | | | | Raw p | Adj p |
|---|---|---|---|---|---|---|---|
| Genbank# | Gene assignment | log2(L/C) | log2(M/C) | log2(S/C) | Log2(A/C) | value | value |
| AA907052 | Unknown | −0.307 | −0.454 | −0.477 | −0.410 | 3.08E−06 | 1.85E−05 |
| AI076295 | MEMO1 locus | −0.547 | −0.518 | −0.553 | −0.540 | 1.09E−04 | 6.54E−04 |
| H25019 | ZZZ3 locus | −0.239 | −0.265 | −0.405 | −0.302 | 2.50E−04 | 0.002 |
| H97875 | Unknown | −0.361 | −0.449 | −0.395 | −0.398 | 6.40E−04 | 0.004 |
| R11217 | Unknown | −0.218 | −0.234 | −0.254 | −0.236 | 6.10E−05 | 3.66E−04 |

L: severely language impaired;
M: mildly affected;
S: with notable savant skills;
A: all autistic groups combined;
C: nonautistic control group.
The adjusted p-value was obtained using a Bonferroni correction for multiple testing.

TABLE 4
Pathway and functional analyses of differentially expressed genes from 3 ASD
subgroups.

| | | |
|---|---|---|
| Severely language-impaired | Mildly autistic | “Savant” group |

Molecular and Cellular Functions (p-value) [#genes]

| | | |
|---|---|---|
| Cell death | Cell growth and proliferation | RNA post-transcriptional modification |
| (1.54E−10-5.99E−03) [83] | (8.70E−05-2.46E−02) [10] | (6.69E−08-4.55E−02) [5] |
| Cellular development | Cellular development | |
| (1.58E−08-5.61E−03) [60] | (2.67E−04-2.19E−02) [11] | |
| Cellular movement | Free radical scavenging | |
| (7.40E−08-5.78E−03) [48] | (4.31E−04-1.62E−02) [4] | |
| Cell growth and proliferation | Cell cycle | |
| (2.00E−06-5.95E−03 [78] | (9.61E−04-2.69E−02) [14] | |
| Cell signaling | Small molecular biochemistry | |
| (1.01E−05-1.79E−03) [32] | (1.13E−03-2.69E−02) [11] | |

Top Canonical Pathways (p-value) [#genes/genes in category]

| | |
|---|---|
| cAMP-mediated signaling | Integrin signaling |
| (3.19E−03) [8/159] | (1.97E−02) [4/192] |
| Hepatic fibrosis/stellate cell activation | Death receptor signaling |
| (4.06E−03) [7/131] | (3.72E−02) [2/61] |
| Hepatic cholestasis | |
| (6.01E−03) [7/162] | |
| B cell receptor signaling | |
| (7.97E−03) [7/148] | |
| (8.6E−03) [7/143] | |

Top Toxicity Lists (p-value) [#genes/genes in category]

| | |
|---|---|
| Hepatic stellate cell activation | Anti-apoptosis |
| (2.74E−04) [5/35] | (1.3E−02) [2/32] |
| Hepatic fibrosis | |
| (3.01E−03) [6/85] | |
| Hepatic cholestasis | |
| (7.66E−03) [7/135] | |
| NF-kB signaling pathway | |
| (1.14E−02) [6/112] | |
| Gene regulation by PPARa | |
| (2.18E−02) [5/95] | |

Ingenuity Pathway Analysis software was used to analyze the gene datasets for functions and pathways that were statistically enriched. The Fisher exact test was used to determine p-values which represent the likelihood that a given function or pathway is identified by chance.

TABLE 5
Neurological functions and disorders associated with differentially
expressed genes from the language and mild ASD subgroups.

Genes

Language vs controls

| | |
|---|---|
| Neurological Disorders | |
| Apoptosis/cell death of neuroglia, | BTG1, QK1, TNFSF10, GAS6, CD44, PTGDS, TLR4, MYC, EDN1, |
| astrocytes, neurons | MAPK1, HDAC9, ITGA2, CREM, FOXO3, MAP1B, TGFB2, CTF1, |
| | ADORA2A, DAPK1, GLRX, SH3RF1, TP53BP2, MAP3K5, BID, |
| | FN1, INSR, MAOA, NOVA1, NR1D1, SH3RF1, IL1RN, PDCD6IP, |
| | APBB1 |
| Catalepsy | ADORA2A, CNR1, PRKAR2B |
| Allodynia | GAL, IL1RN, PRKCG, PTGDS, TLR4 |
| Seizures (mice) | GAL, IL1RN |
| Hypernociception | EDN1, IL15, IL1RN |
| Nervous system functions | |
| Circadian rhythm | AANAT, BHLHB2, BHLHB3, CLOCK, CREM, CRY1, DPYD, |
| | MAPK1, NPAS2, NR1D1, PER1, PER3, PTGDS |
| Generation of neuronal | ASCL1, CNR1 |
| progenitors | |
| Neurological process | CTF1, GAL, NR3C1, PLAU, SERPINE2, ADORA2A, ASCL1, |
| Olfactory memory | CNR1, CREM, CYBB, GM2A, GNAI1, NOVA1, OPHN1, |
| | PRKAR2B, PRKCG |
| | GAL, PLAU |

Mild vs controls

| | |
|---|---|
| Neurological Disorders | |
| Atrophy of dendrites | PRNP |
| Neurological deficit of mice | ADORA2A |
| Gliosis of cerebellum | PRNP |
| Gliosarcoma | MGMT |
| Nervous system functions | |
| Outgrowth of neurites | ADORA2A, MARCKS (includes EG: 4082), OMG, PRNP, SLIT2 |
| Migration of neuroglia | PRNP, SLIT2 |
| Differentiation of microglia | ITGAM |
| Branching of neurites | FNBP1, SLIT2 |

Each dataset containing significantly differentially expressed genes from a 2-class SAM for each of the subgroups was analyzed using Ingenuity Pathway Analysis network prediction software, using an expression cutoff of log₂(ratio) of 0.3 for functional/pathway analyses. Fisher Exact p-values for enrichment of genes associated with the specified disorders and functions were <0.02.

TABLE 6
Quantitative RT-PCR confirmation of 6 of the circadian rhythm genes

| Gene | qPCR log2 | SE | Microarray log2 | SE |
|---|---|---|---|---|
| AANAT | −2.115 | 0.31 | −0.468 | 0.03 |
| BHLHB2 | 0.913 | 0.28 | 0.851 | 0.12 |
| CRY1 | 1.202 | 0.36 | 0.865 | 0.10 |
| DPYD | −2.135 | 0.79 | −1.080 | 0.52 |
| NPAS2 | −0.350 | 0.70 | −0.657 | 0.08 |
| PER3 | −1.279 | 0.52 | −1.102 | 0.27 |

Five representative samples were selected from the control and severely language impaired groups for qRT-PCR analyses (in triplicate) of AANAT, BHLHB2, CRY1, DPYD, NPAS2, and PER3. The average expression values from microarray and qRT-PCR analyses are shown for comparison along with the standard error of the mean (SE) for each set of analyses on the representative samples.

TABLE 7
Significant differentially expressed genes (FDR <5%) from a
2-class SAM analysis of DNA microarray data from combined
autistic samples (87 cases) vs. controls (29 subjects) with mean
log2(ratio) ≦−0.29.

| | Gene | |
|---|---|---|
| Genbank# | Symbol | log2(ratio) |
| AI018127 | unknown | −0.97 |
| AI218398 | unknown | −0.63 |
| AA446651 | SH3D19 | −0.55 |
| T65857 | unknown | −0.53 |
| T84782 | unknown | −0.50 |
| N47010 | KIAA1432 | −0.47 |
| H10156 | unknown | −0.47 |
| AA412435 | unknown | −0.45 |
| AA156946 | KLF6 | −0.44 |
| AA939251 | unknown | −0.43 |
| AA707219 | ELL2 | −0.43 |
| AI076295 | C2ORF4 | −0.43 |
| AA907052 | unknown | −0.43 |
| R89313 | UGCGL1 | −0.41 |
| AI820599 | DNASE2B | −0.41 |
| AA490903 | PSCDBP | −0.40 |
| AA609962 | ITGAM | −0.40 |
| N95440 | unknown | −0.40 |
| T59442 | unknown | −0.39 |
| T97353 | PFTK1 | −0.39 |
| AA013481 | unknown | −0.38 |
| H21071 | NAIP | −0.38 |
| AA902164 | CCDC50 | −0.37 |
| H30558 | ERO1LB | −0.37 |
| H19429 | ERO1LB | −0.36 |
| H56961 | JMJD2C | −0.36 |
| AI091450 | SYTL3 | −0.36 |
| AA704941 | LARP5 | −0.35 |
| AI028039 | VPS13C | −0.35 |
| AA436187 | ITGAM | −0.35 |
| AA883496 | SFRS10 | −0.35 |
| AA111979 | KLHL24 | −0.35 |
| AI092008 | LRP2BP | −0.34 |
| AA400474 | ZPBP | −0.34 |
| H48346 | TMEM23 | −0.34 |
| AA456112 | ACTR3 | −0.34 |
| AA865224 | KLF6 | −0.33 |
| N80451 | unknown | −0.33 |
| H05653 | unknown | −0.33 |
| AA626236 | UBE2E2 | −0.33 |
| R39926 | GPR137B | −0.33 |
| R89715 | PRKCG | −0.33 |
| H97875 | MGC24039 | −0.33 |
| N65982 | unknown | −0.33 |
| AA975530 | SSH2 | −0.33 |
| AA995108 | CUL3 | −0.33 |
| AA970158 | unknown | −0.33 |
| R07066 | C2ORF32 | −0.33 |
| AA019547 | SND1 | −0.32 |
| H48138 | LOC145474 | −0.32 |
| AI248021 | HLF | −0.32 |
| AA286777 | PHC3 | −0.32 |
| AA922231 | unknown | −0.32 |
| R20547 | BHLH89 | −0.32 |
| AI127342 | unknown | −0.32 |
| AI093876 | GABPB2 | −0.32 |
| N72150 | unknown | −0.32 |
| AA977210 | FAF1 | −0.32 |
| T99772 | unknown | −0.32 |
| AI001741 | NFKB1 | −0.31 |
| H37761 | NR4A3 | −0.31 |
| AI222606 | unknown | −0.31 |
| AA699707 | FNBP1 | −0.31 |
| AA045278 | SART2 | −0.31 |
| R56829 | MASP2 | −0.31 |
| AA120875 | EPC1 | −0.31 |
| H13205 | IDS | −0.31 |
| AI028234 | RHOA | −0.31 |
| N67598 | DST | −0.31 |
| AA281729 | ARL5B | −0.30 |
| T95898 | FLJ43663 | −0.30 |
| AA001219 | SOCS3 | −0.30 |
| AA455248 | STK4 | −0.30 |
| AA928817 | ZNF6 | −0.30 |
| H73587 | unknown | −0.30 |
| AA416628 | KLF6 | −0.30 |
| AA026388 | SENP6 | −0.30 |
| AA677106 | RAB2 | −0.30 |
| H92525 | CDC2L6 | −0.29 |
| AA677280 | SPRED1 | −0.29 |
| AA017242 | ZNF407 | −0.29 |
| N45223 | TSC22D2 | −0.29 |
| H96791 | BIN3 | −0.29 |
| H54779 | EPC1 | −0.29 |
| AI336948 | BACH1 | −0.29 |
| AA905165 | unknown | −0.29 |
| N48820 | GABPB2 | −0.29 |
| AA005196 | ZNF138 | −0.29 |
| R16146 | PFKRB2 | −0.29 |
| AI287588 | RAPGEF1 | −0.29 |

TABLE 8
Significant differentially expressed genes (FDR <0.0000%) from a
2-class SAM analysis of DNA microarray data from autistic samples
(31 cases) with severe language impairment (L subgroup) vs. controls
(29 subjects) with mean log2(ratio) ≧±0.29.

| | Gene | |
|---|---|---|
| Genbank# | symbol | log2(ratio) |
| R38090 | C11ORF41 | 1.20 |
| H19227 | ST3GAL6 | 1.18 |
| AI371096 | DAPK1 | 0.93 |
| AA418748 | LOC389831 | 0.84 |
| AA865590 | BCAT1 | 0.82 |
| AA991950 | unknown | 0.82 |
| AA150422 | CYBRD1 | 0.80 |
| AA418546 | CD109 | 0.77 |
| AA490486 | unknown | 0.74 |
| AA461071 | SLC23A2 | 0.74 |
| AA455945 | TSPO | 0.73 |
| R55334 | CROCCL2 | 0.72 |
| AA478730 | unknown | 0.71 |
| H79047 | IGFBP2 | 0.71 |
| AA971895 | unknown | 0.70 |
| AI240359 | unknown | 0.70 |
| N69689 | RAB1A | 0.68 |
| AI198650 | unknown | 0.67 |
| AA702797 | KLHL6 | 0.67 |
| T90980 | unknown | 0.67 |
| AA455350 | DFNA5 | 0.67 |
| AA598781 | IRF2BP2 | 0.66 |
| R54846 | FGFR1 | 0.65 |
| AI087951 | unknown | 0.61 |
| T99645 | KCTD5 | 0.61 |
| N26163 | LOC389831 | 0.58 |
| N70654 | unknown | 0.55 |
| W02016 | unknown | 0.55 |
| R56082 | SV2B | 0.55 |
| W74070 | ABCA8 | 0.54 |
| AA699790 | RPL31 | 0.54 |
| AA857705 | LOC401131 | 0.54 |
| AI018016 | LOC401089 | 0.54 |
| AA960789 | unknown | 0.53 |
| W19228 | unknown | 0.53 |
| AA194143 | KRCC1 | 0.53 |
| T91078 | LOC401321 | 0.52 |
| AI076602 | unknown | 0.52 |
| AA664377 | unknown | 0.52 |
| W93120 | unknown | 0.51 |
| AA127069 | TMEM158 | 0.50 |
| R93719 | GSPT1 | 0.49 |
| AA491292 | SLC39A10 | 0.49 |
| H14231 | unknown | 0.49 |
| T97599 | DTX1 | 0.49 |
| AI288235 | FLJ35282 | 0.48 |
| AI141972 | MARCH6 | 0.48 |
| AA071470 | WWC3 | 0.47 |
| AA886999 | ZNF197 | 0.47 |
| N69252 | unknown | 0.46 |
| R81831 | ZNF217 | 0.46 |
| AA705942 | HOOK3 | 0.45 |
| AA262235 | INTS6 | 0.45 |
| N45114 | ZNF322A | 0.45 |
| R56894 | MARK1 | 0.45 |
| AA977196 | TMEM38A | 0.45 |
| AI187812 | unknown | 0.44 |
| AI248260 | unknown | 0.43 |
| AA908241 | unknown | 0.43 |
| H22949 | unknown | 0.43 |
| N72256 | ZADH2 | 0.43 |
| AI074217 | unknown | 0.43 |
| AI301365 | LOC389833 | 0.43 |
| AA634028 | HLA-DPA1 | 0.42 |
| AI125886 | unknown | 0.42 |
| H96982 | TRIM13 | 0.42 |
| AA909676 | PVT1 | 0.42 |
| AI032307 | unknown | 0.41 |
| N77198 | unknown | 0.41 |
| AA975183 | THEM4 | 0.41 |
| H57273 | PRCP | 0.41 |
| AA620472 | unknown | 0.41 |
| R51386 | unknown | 0.40 |
| W07745 | ZADH2 | 0.40 |
| R39745 | unknown | 0.40 |
| AA279467 | RPL23AP7 | 0.40 |
| AA910213 | ALS2CL | 0.40 |
| AA873427 | unknown | 0.39 |
| R37119 | unknown | 0.39 |
| AA916872 | unknown | 0.38 |
| H79035 | HOMEZ | 0.38 |
| AA450332 | unknown | 0.38 |
| R01246 | unknown | 0.38 |
| T68845 | DEXI | 0.38 |
| AA663944 | TRIM4 | 0.38 |
| AI050027 | unknown | 0.37 |
| AA476584 | MGC12966 | 0.37 |
| AI003774 | unknown | 0.37 |
| H06377 | unknown | 0.37 |
| N29986 | LHFPL3 | 0.37 |
| T52700 | KIAA1161 | 0.36 |
| T59422 | unknown | 0.36 |
| R53951 | PDCD6 | 0.36 |
| AA626146 | RPS24 | 0.36 |
| R89365 | AMN1 | 0.35 |
| W86452 | unknown | 0.35 |
| AA424756 | NUFIP2 | 0.35 |
| AI264427 | FLJ38028 | 0.35 |
| AA664004 | TPP1 | 0.35 |
| AA921942 | unknown | 0.35 |
| H14604 | PANK1 | 0.35 |
| AA071526 | PPP1R10 | 0.35 |
| N25657 | unknown | 0.34 |
| H15844 | EP400NL | 0.34 |
| W31566 | unknown | 0.33 |
| AA872279 | FNBP4 | 0.33 |
| AA455126 | ATP5G2 | 0.33 |
| W88562 | C14ORF119 | 0.32 |
| R26811 | unknown | 0.32 |
| AA779937 | EEPD1 | 0.32 |
| N27415 | TRIM4 | 0.31 |
| AA778640 | NPEPL1 | 0.31 |
| H16725 | NAT13 | 0.31 |
| R37598 | unknown | 0.30 |
| AA886236 | RSBN1L | 0.30 |
| AA934126 | LARGE | 0.30 |
| N68510 | BRD3 | 0.29 |
| R30960 | unknown | 0.29 |
| H96554 | unknown | 0.29 |
| AA777765 | C10ORF12 | −0.29 |
| AA465166 | CCNL1 | −0.29 |
| AA934401 | unknown | −0.29 |
| AI018042 | unknown | −0.30 |
| AI341901 | SPHK1 | −0.30 |
| AI219775 | ANKRD11 | −0.30 |
| AA677078 | REEP5 | −0.30 |
| AA906896 | TATDN3 | −0.31 |
| AA045665 | ALG13 | −0.31 |
| H60119 | EHBP1 | −0.31 |
| AI248210 | UBE2A | −0.31 |
| AI160166 | PPIA | −0.31 |
| AA676649 | TSHZ2 | −0.32 |
| AA426120 | TRIM33 | −0.32 |
| AI031771 | unknown | −0.32 |
| N52605 | PPP1R2 | −0.32 |
| AA906454 | C14ORF108 | −0.32 |
| AA778856 | MICAL2 | −0.33 |
| AI348442 | C5ORF5 | −0.33 |
| N72196 | NR4A3 | −0.34 |
| H72937 | DECR1 | −0.34 |
| AI222165 | PABPC1 | −0.34 |
| R38639 | HDHD1A | −0.34 |
| AA678065 | BPGM | −0.34 |
| AA453477 | XPNPEP1 | −0.34 |
| AA933721 | MTMR2 | −0.34 |
| AA291183 | RSRC2 | −0.34 |
| AI583623 | SFRS10 | −0.35 |
| R24969 | GABRB1 | −0.35 |
| AA497132 | PSMD12 | −0.35 |
| AA862434 | PSMB9 | −0.36 |
| AA399952 | USP50 | −0.36 |
| T55592 | HNRPD | −0.36 |
| AA703378 | unknown | −0.36 |
| N25798 | ANKRD28 | −0.36 |
| AA878762 | unknown | −0.36 |
| AI733697 | C12ORF30 | −0.36 |
| AA984679 | unknown | −0.37 |
| N72288 | MARCH7 | −0.37 |
| AA284634 | JAK1 | −0.37 |
| AA922097 | C2ORF34 | −0.37 |
| AA677280 | SPRED1 | −0.37 |
| AA136060 | PCGF5 | −0.37 |
| AA504356 | PCBP2 | −0.38 |
| AA777255 | ZC3H15 | −0.38 |
| AA876421 | PPP1CB | −0.38 |
| AA504273 | ZNF514 | −0.39 |
| AA452545 | SGTB | −0.39 |
| H73594 | unknown | −0.39 |
| AI246463 | unknown | −0.39 |
| AI266442 | TMEM140 | −0.39 |
| N76276 | unknown | −0.39 |
| R06605 | PTPN1 | −0.39 |
| AA609738 | HNRPD | −0.40 |
| AA456821 | NETO2 | −0.40 |
| AI209205 | RSRC2 | −0.41 |
| H82104 | HNRPD | −0.41 |
| N51323 | BTG1 | −0.41 |
| N36389 | KIAA0226 | −0.42 |
| AI290596 | RAB30 | −0.42 |
| AI028234 | RHOA | −0.42 |
| AA455970 | RNF139 | −0.43 |
| R26614 | unknown | −0.43 |
| R95732 | TRDMT1 | −0.43 |
| H44784 | DST | −0.43 |
| H56961 | JMJD2C | −0.43 |
| H54779 | EPC1 | −0.44 |
| AA281137 | USP6NL | −0.44 |
| AA416760 | unknown | −0.44 |
| AA281729 | ARL5B | −0.44 |
| AA443846 | PDLIM5 | −0.44 |
| N73031 | C1GALT1 | −0.44 |
| AA626724 | CREM | −0.44 |
| AA018569 | unknown | −0.45 |
| AI076295 | MEMO1 | −0.45 |
| N48701 | ELK3 | −0.45 |
| N48820 | GABPB2 | −0.45 |
| AA205598 | WDR72 | −0.46 |
| AA456112 | ACTR3 | −0.46 |
| R56829 | MASP2 | −0.46 |
| AA704941 | LARP5 | −0.46 |
| W92859 | PTPN1 | −0.47 |
| AA017242 | ZNF407 | −0.47 |
| AI093876 | unknown | −0.47 |
| AA206614 | MCTP2 | −0.47 |
| AI244972 | TRIB1 | −0.47 |
| N65982 | unknown | −0.47 |
| AA210701 | MOBKL1B | −0.47 |
| N67051 | PTEN | −0.47 |
| H99054 | RAB30 | −0.47 |
| N72150 | unknown | −0.48 |
| T50828 | CASP7 | −0.48 |
| AA705081 | unknown | −0.49 |
| AA005196 | ZNF138 | −0.49 |
| AA013481 | unknown | −0.50 |
| AA120875 | EPC1 | −0.51 |
| H48346 | SGMS1 | −0.51 |
| AA286777 | PHC3 | −0.51 |
| W91960 | SSBP3 | −0.51 |
| AI018099 | KRT18P42 | −0.52 |
| AA610081 | SLC16A1 | −0.53 |
| AA488969 | RAPGEF2 | −0.53 |
| AI492016 | JAK1 | −0.55 |
| AA707219 | ELL2 | −0.56 |
| AA883496 | SFRS10 | −0.56 |
| T97353 | PFTK1 | −0.57 |
| AI250784 | TOX | −0.57 |
| T59442 | unknown | −0.57 |
| AA436187 | ITGAM | −0.58 |
| N47010 | KIAA1432 | −0.58 |
| N80451 | unknown | −0.59 |
| AA609962 | ITGAM | −0.59 |
| AA111979 | KLHL24 | −0.59 |
| AA416628 | KLF6 | −0.60 |
| H40023 | EIF5 | −0.61 |
| AA045278 | DSE | −0.61 |
| H10156 | unknown | −0.61 |
| AA015658 | ARRDC3 | −0.62 |
| N95440 | unknown | −0.62 |
| AA406020 | ISG15 | −0.63 |
| AA865224 | KLF6 | −0.65 |
| AA902164 | CCDC50 | −0.67 |
| AA022908 | RAPGEF2 | −0.68 |
| AA156946 | KLF6 | −0.69 |
| AA453293 | PDE4B | −0.70 |
| T65857 | unknown | −0.72 |
| T84782 | unknown | −0.73 |
| AA430512 | SERPINB9 | −0.75 |
| AA279883 | CD69 | −0.79 |
| H54629 | TNFSF10 | −0.86 |
| AA446651 | SH3D19 | −0.90 |
| R33609 | ARRDC3 | −0.95 |
| AA972030 | RALGPS2 | −1.16 |
| R44985 | C20ORF103 | −1.24 |

TABLE 9
Significant differentially expressed genes (FDR <5%) from a 2-class
SAM analysis of DNA microarray data from autistic subjects (26 cases)
with mild phenotype (M subgroup) vs. controls (29 subjects) with mean
log2(ratio) ≧±0.3.

| | Gene | |
|---|---|---|
| Genbank# | symbols | log2(ratio) |
| AA176957 | NEB | 0.69 |
| W24622 | YAP1 | 0.59 |
| AI198213 | RNU12P | 0.52 |
| AI367109 | SPI1 | 0.47 |
| AI240359 | unknown | 0.47 |
| AA455969 | PRNP | 0.47 |
| AI283175 | unknown | 0.45 |
| R59187 | ZNF740 | 0.43 |
| AA487034 | TGFBR2 | 0.42 |
| AA777898 | unknown | 0.41 |
| AA172076 | TMC5 | 0.40 |
| AA916872 | unknown | 0.39 |
| AA401111 | GPI | 0.39 |
| H99639 | MAX | 0.38 |
| AI018016 | LOC401089 | 0.36 |
| H14231 | unknown | 0.35 |
| AA482328 | MARCKS | 0.34 |
| R68630 | unknown | 0.34 |
| AA864677 | unknown | 0.34 |
| AA779937 | EEPD1 | 0.34 |
| AA284243 | ZBTB4 | 0.34 |
| H82273 | FEM1B | 0.34 |
| AA620783 | NA | 0.33 |
| AA164630 | MINA | 0.33 |
| H25895 | unknown | 0.32 |
| H17635 | TNKS2 | 0.32 |
| W07745 | ZADH2 | 0.32 |
| AA621367 | KCTD7 | 0.31 |
| H79979 | TRUB1 | 0.31 |
| AA009830 | SBNO1 | 0.31 |
| H44956 | FAH | 0.31 |
| W42459 | PYROXD1 | 0.30 |
| AI074217 | unknown | 0.30 |
| H65596 | SAP18 | 0.30 |
| H78999 | DCBLD2 | 0.30 |
| AA630097 | unknown | 0.29 |
| AA459383 | MED13 | 0.29 |
| H95716 | USPL1 | 0.29 |
| AA054950 | VPS41 | −0.29 |
| R01415 | SNAPC3 | −0.29 |
| AA704425 | GMDS | −0.29 |
| H65481 | NA | −0.29 |
| AA683336 | KIAA0922 | −0.29 |
| AI287588 | RAPGEF1 | −0.29 |
| AA682408 | VGLL4 | −0.29 |
| AA704934 | ABI1 | −0.29 |
| AA873459 | WTAP | −0.29 |
| H90893 | CDC73 | −0.29 |
| AI242970 | PRDM2 | −0.30 |
| AI309927 | CENTA1 | −0.30 |
| AI276822 | unknown | −0.30 |
| AI027259 | C12ORF56 | −0.30 |
| AI280997 | FANCC | −0.30 |
| AA678124 | EGFR | −0.30 |
| AI028234 | RHOA | −0.30 |
| R84636 | SSH2 | −0.30 |
| T97887 | unknown | −0.30 |
| AI140549 | BRE | −0.30 |
| AI076698 | LOC92017 | −0.30 |
| AI001741 | NFKB1 | −0.30 |
| H18953 | LCOR | −0.30 |
| T97112 | WDR37 | −0.30 |
| W95003 | AKAP13 | −0.30 |
| AI733611 | unknown | −0.30 |
| AI076577 | EEF1G | −0.30 |
| H48138 | LOC145474 | −0.30 |
| AI127072 | C5ORF28 | −0.30 |
| H18668 | VTI1A | −0.30 |
| H11737 | MAP4 | −0.31 |
| N72288 | unknown | −0.31 |
| N80619 | ATRN | −0.31 |
| AA700415 | SSBP3 | −0.31 |
| T78110 | CCDC52 | −0.32 |
| AI278663 | KIAA0368 | −0.32 |
| AA677880 | MBD2 | −0.32 |
| H71242 | RERE | −0.32 |
| N72150 | unknown | −0.32 |
| AI005358 | ZNF768 | −0.32 |
| AI217765 | AKTIP | −0.32 |
| AI335086 | ANGPTL3 | −0.32 |
| AA917005 | unknown | −0.32 |
| H54779 | EPC1 | −0.32 |
| AI028699 | NIPBL | −0.32 |
| AA610081 | SLC16A1 | −0.33 |
| AI290596 | RAB30 | −0.33 |
| AA099394 | SSR1 | −0.33 |
| AA436187 | ITGAM | −0.33 |
| H66005 | TSPAN9 | −0.33 |
| T78451 | LOC440353 | −0.33 |
| N76276 | unknown | −0.33 |
| AI289840 | ADORA2A | −0.33 |
| T53389 | FCGBP | −0.33 |
| AA677601 | NR5A2 | −0.33 |
| AA045179 | MED17 | −0.34 |
| N47511 | OMG | −0.34 |
| AI248342 | CDYL | −0.34 |
| AI290481 | PTBP2 | −0.34 |
| AI246463 | unknown | −0.34 |
| N80451 | unknown | −0.34 |
| N24004 | MUTYH | −0.34 |
| AI147399 | RPAP2 | −0.34 |
| AI240309 | DCHS2 | −0.34 |
| AA995108 | CUL3 | −0.34 |
| T70401 | unknown | −0.34 |
| N54917 | unknown | −0.34 |
| R08275 | ZNRF3 | −0.34 |
| T57841 | UFD1L | −0.34 |
| H37761 | NR4A3 | −0.34 |
| AA703625 | TMEM16F | −0.34 |
| R51261 | NSMCE2 | −0.34 |
| AA666405 | PDCD11 | −0.34 |
| AI138374 | TAS2R14 | −0.35 |
| AI290868 | SPHKAP | −0.35 |
| AA774645 | EPN2 | −0.35 |
| AA156424 | MCPH1 | −0.35 |
| AI248501 | MGMT | −0.35 |
| AA708789 | unknown | −0.35 |
| AI198170 | FAF1 | −0.35 |
| AI279944 | MS4A6E | −0.35 |
| AA994835 | CRIM1 | −0.36 |
| R15735 | unknown | −0.36 |
| AA922231 | NA | −0.36 |
| H48346 | SGMS1 | −0.36 |
| N67598 | DST | −0.36 |
| AI201264 | SLC20A2 | −0.36 |
| H51984 | RHBDD1 | −0.37 |
| N45223 | TSC22D2 | −0.37 |
| AI268113 | unknown | −0.37 |
| AA969014 | RAPGEF1 | −0.37 |
| AI269386 | ABCB5 | −0.38 |
| AI219977 | KLHL9 | −0.38 |
| AA938900 | LY9 | −0.38 |
| H63223 | EXT1 | −0.38 |
| AA626236 | UBE2E2 | −0.39 |
| N55105 | LOC440353 | −0.39 |
| AA707544 | ZMYM2 | −0.39 |
| AA699707 | FNBP1 | −0.39 |
| AA705219 | LOC440345 | −0.40 |
| AA936169 | MYO1E | −0.40 |
| AA897665 | TRIO | −0.40 |
| H92525 | CDC2L6 | −0.40 |
| H13205 | IDS | −0.40 |
| AI076295 | MEMO1 | −0.40 |
| H97875 | MGC24039 | −0.41 |
| H68312 | JMJD2C | −0.42 |
| AA489463 | SLIT2 | −0.42 |
| AI264565 | MEI1 | −0.42 |
| AI307137 | unknown | −0.42 |
| H19429 | ERO1LB | −0.43 |
| AI198871 | PBX1 | −0.44 |
| T84782 | unknown | −0.45 |
| AI243860 | unknown | −0.45 |
| AA644224 | CHD7 | −0.45 |
| H80712 | CASP10 | −0.45 |
| AA975530 | SSH2 | −0.45 |
| AA677106 | RAB2A | −0.45 |
| AI248213 | FLJ43663 | −0.46 |
| AA907052 | unknown | −0.46 |
| H21670 | RAB18 | −0.47 |
| AA609962 | ITGAM | −0.48 |
| AI291305 | AFTPH | −0.48 |
| AI122689 | unknown | −0.49 |
| AI266442 | TMEM140 | −0.50 |
| AI242955 | unknown | −0.51 |
| R07066 | CNRIP1 | −0.52 |
| AI214443 | unknown | −0.56 |
| H21071 | unknown | −0.60 |
| AA939251 | unknown | −0.60 |
| R25895 | unknown | −0.73 |

TABLE 10
Significant differentially expressed genes (FDR <5%) from a 2-class SAM
analysis of DNA microarray data from autistic subjects (30 cases) with
savant phenotype (S subgroup) vs. controls (29 subjects) with mean
log2(ratio) <−0.29.

| Genbank# | Gene symbol | log2(ratio) |
|---|---|---|
| AA907052 | unknown | −0.51 |
| T99772 | unknown | −0.46 |
| AA026388 | SENP6 | −0.46 |
| AI076295 | MEMO1 | −0.45 |
| H29771 | ATF6 | −0.44 |
| AA922231 | unknown | −0.42 |
| AA156946 | KLF6 | −0.41 |
| T84663 | unknown | −0.41 |
| T95898 | FLJ43663 | −0.41 |
| AI222606 | unknown | −0.41 |
| AA937453 | FLJ43663 | −0.40 |
| AA906961 | unknown | −0.40 |
| R06688 | unknown | −0.39 |
| AA013481 | unknown | −0.39 |
| AA447768 | HRB | −0.38 |
| AA019547 | SND1 | −0.38 |
| AA677828 | unknown | −0.37 |
| R06119 | unknown | −0.37 |
| H47334 | unknown | −0.37 |
| AA972308 | FLJ43663 | −0.37 |
| AA284267 | unknown | −0.37 |
| H25019 | ZZZ3 | −0.37 |
| AA426028 | PSIP1 | −0.37 |
| H56961 | JMJD2C | −0.36 |
| AI005270 | unknown | −0.36 |
| N72540 | unknown | −0.36 |
| AA443140 | KIFC2 | −0.35 |
| H97875 | MGC24039 | −0.35 |
| AI218740 | MARK3 | −0.35 |
| AA905165 | unknown | −0.35 |
| AA977210 | FAF1 | −0.35 |
| AA626383 | NRD1 | −0.34 |
| AA777799 | ALAD | −0.34 |
| AA620961 | GNG2 | −0.34 |
| AA620393 | STIM2 | −0.33 |
| AA954583 | unknown | −0.33 |
| AA927612 | unknown | −0.33 |
| AA971762 | unknown | −0.33 |
| AA398234 | C16ORF72 | −0.32 |
| AA677601 | NR5A2 | −0.32 |
| AA923359 | XPO6 | −0.32 |
| AI073491 | PHKB | −0.32 |
| AA934368 | RP11- | −0.32 |
| | 298P3.3 | |
| AA917778 | USP3 | −0.31 |
| AI138734 | RNF13 | −0.31 |
| AA598548 | unknown | −0.30 |
| AA704941 | LARP5 | −0.30 |
| R96240 | SFPQ | −0.30 |
| AA912705 | SP3 | −0.30 |
| AA777827 | PSIP1 | −0.30 |
| AI085796 | PSMD1 | −0.30 |
| AI004821 | unknown | −0.29 |
| AA455164 | SFRS1 | −0.29 |
| AI299893 | SFRS12 | −0.29 |

TABLE 11
Sequences of primers used for qRT-PCR analyses
(“Forward Primer” sequences disclosed as SEQ
ID NOS 1-11 and “Reverse Primer” sequences
disclosed as SEQ ID NOS 12-22, all
respectively, in order of appearance.)

| Gene | Forward Primer | Reverse Primer |
|---|---|---|
| Symbol | (5′ --> 3′) | (5′ --> 3′) |
| AANAT | GTCCCGGATTT | CCAGCTTTGGA |
| | TACTGGTTC | AGTGTCCTC |
| BHLHB2 | GTACCTCCAGG | CCACTGTCTGT |
| | AAGCCATCA | GTCCGTGTC |
| CRY1 | GGTGGGAAACG | TGCCTCAAGAT |
| | TCCTAGTCA | TTTCTGGTTT |
| DPYD | GAAAACGGCTG | GCAAGTTCCGT |
| | CATATTGGT | CCAGTCATT |
| ITGAM | ATCAGGTGGTG | GTCTGTCTGCG |
| | AAAGGCAAG | TGTGCTGTT |
| MBD2 | GAGACTGCGAA | CATTCCAAGCA |
| | ACGATCCTC | GAGCAAACA |
| NFKB1 | AACCACAGAGC | GCAAGCTGCAT |
| | AAGATCAGGA | AGCCTTCTC |
| NPAS2 | GCATGTTCCAG | GCTGCAGGAAC |
| | ACCATCAAA | ATCTGGAC |
| PER3 | AAAGGAGGAGC | ACCAGAACCTG |
| | TGGCTAAGG | ACCACAGGA |
| RHOA | AGTCCACGGTC | AGGCTCCATCA |
| | TGGTCTTCA | CCAACAATC |
| SLIT2 | TGACCCTTGCC | CATCACAGAGG |
| | TTGGAAATA | ACACCTCCA |

### Example 3

### Gene Expression Profiling of Lymphoblastoid Cell Lines from Autistic and Nonaffected Sib Pairs Reveals Altered Signaling and Metabolic Pathways Relevant to Development and Steroid Biosynthesis

In this Example, the gene expression profiles of LCL derived from 21 sib pairs where one of the siblings is autistic and the other is not were analyzed. To reduce the phenotypic heterogeneity among the samples, cell lines were selected from individuals who presented with severe language impairment as reflected by scores on the Autism Diagnostic Interview-Revised (ADIR) questionnaire, as described in Materials and Methods. Results from gene expression analysis of LCL from these individuals revealed alterations in genes involved in cholesterol metabolism and steroid hormone biosynthesis, as well as genes involved in neuronal processes and development. A steroid profile of cell extracts using HPLC-tandem mass spectrometry methods further confirmed elevations in testosterone levels in the autistic sibling.

### Methods:

### Cell Culture

Lymphoblastoid cell lines (LCL) derived from lymphocytes of autistic and normal siblings were obtained from the Autism Genetic Resource Exchange (AGRE) and cultured in RPMI 1640 with 15% fetal bovine serum and antibiotics. Lymphocyte donors all provided written consent to AGRE which states that the samples and the derived cell lines will be used indefinitely by scientists who are qualified and approved by AGRE.

### Selection of Samples

To reduce the heterogeneity of the samples for gene expression analyses, we used a novel clustering procedure to identify phenotypically distinct groups of individuals on the basis of severity associated with 123 items on the Autism Diagnostic Interview-Revised scoresheets. This procedure, described in Example 1 supra, resulted in separation of the autistic individuals into 4 phenotypic subgroups. For this study, autistic male individuals were selected from the subgroup associated with severe language impairment, each of whom had a male sibling who was not affected by autism who served as a control in a paired statistical analysis of gene expression data derived from LCL of the respective siblings. To further reduce the heterogeneity within the samples and eliminate confounding factors due to co-existing conditions or known genetic abnormalities, LCL from females, individuals with specific genetic and chromosomal abnormalities (e.g., Fragile X, chromosome 15q11-q13 duplication) and with diagnosed co-morbid disorders (e.g., bipolar disorder, obsessive compulsive disorder), and those born prematurely (<35 weeks of gestation) were excluded from this study.

### DNA Microarray Analysis

RNA was isolated from LCL 3 days after tissue culture using TRIzol Reagent (Invitrogen) according to the manufacturer's protocol. Fluorescently labeled sample cDNA was obtained by incorporation of amino-allyl dUTP during first-strand synthesis, followed by coupling to the ester of Cyanine (Cy)-3 as previously described [Hu V W, Frank B C, Heine S, Lee N H, Quackenbush J. (2006)]. Stratagene Universal human reference RNA was used as a common reference RNA sample for all hybridizations, in which the reference cDNA was labeled with Cy-5 dye. For two-color DNA microarray analyses, sample and reference cDNA were co-hybridized onto a custom printed microarray containing 39,936 human PCR amplicon probes derived from cDNA clones purchased from Research Genetics (Invitrogen). After hybridization and washing according to published procedures [Hu V W, Frank B C, Heine S, Lee N H, Quackenbush J. (2006)], the microarrays were scanned for fluorescence signals using a Genepix 4000B laser scanner. Normalized gene expression levels were derived from the resulting image files using TIGR SpotFinder, MIDAS, and MeV analysis programs which are all part of the TM4 Microarray Analysis Software Package available at www.tm4.org. Within MeV, the Significance Analysis of Microarray (SAM) module [Tusher V G, Tibshirani R, Chu G. (2001)] was employed to obtain statistically significant differentially expressed genes using a one-class SAM analysis of the log₂ ratios of relative expression data from the autistic and nonautistic sib pairs.

### Quantitative PCR Analysis

The expression levels of select genes that were significantly differentially expressed within the stated FDR were further quantified by real time RT-PCR on an ABI Prism 7300 Sequence Detection System using Invitrogen's Platinum SYBR Green qPCR SuperMix-UDG with ROX. These included genes involved in cholesterol and steroid hormone metabolism as well as genes implicated in development and autism. Total RNA (same preparations used in microarray analyses) was reverse transcribed into cDNA using the iScript cDNA Synthesis Kit (Bio-Rad, Hercules, Calif.). Briefly, 1 μg of total RNA was added to a 20 μl reaction mix containing reaction buffer, magnesium chloride, dNTPs, an optimized blend of random primers and oligo(dT), an RNase inhibitor and a MMLV RNase H+ reverse transcriptase. The reaction was incubated at 25° C. for 5 minutes followed by 42° C. for 30 minutes and ending with 85° C. for 5 minutes. The cDNA reactions were then diluted to a volume of 50 μl with water and used as a template for quantitative PCR.

PCR primers for genes identified by microarray analysis as differentially expressed were selected for specificity by the National Center for Biotechnology Information Basic Local Alignment Search Tool (NCBI BLAST) of the human genome, and amplicon specificity was verified by first-derivative melting curve analysis with the use of software provided by PerkinElmer (Emeryville, Calif.) and Applied Biosystems. Sequences of primers used for the real-time RT-PCR are given in Table 20.

Quantitative RT-PCR was performed on all samples from the sib pair analyses, with quantification and normalization of relative gene expression using universal 18S rRNA primers, with samples normalized to their 18S rRNA standard curves. For additional confirmation, the expression levels of some genes in representative samples were quantified using the comparative threshold cycle method as described previously [Letwin N E, Kafkafi N, Benjamini Y, Mayo C, Frank B C, et al. (2006)]. The expression of the “housekeeping” genes MDH1 (NM_{—}005917), ARF1 (NM_{—}001024227) and ACSL5 (NM_{—}016234) were used for normalization as these genes did not exhibit differential expression in our microarray assays. The qPCR reactions were done in duplicate or triplicate.

### Pathway and Functional Analyses

The datasets of differentially expressed genes between autistic probands and unaffected siblings were analyzed using Ingenuity Pathway Analysis and Pathway Studio 5 to identify relational gene networks, high level functions, and small molecules associated with the gene regulatory networks. DAVID Bioinformatics Resources (david.abcc.ncifcrf.gov) was also utilized for additional functional annotation and relevant pathways represented within the gene datasets [Dennis, G., Jr, Sherman B T, Hosack D A, Yang J, Gao W, et al. (2003)].

### Metabolic Profiling of Steroid Hormones in LCL

Metabolites were extracted from LCL using acetonitrile and analyzed by isotope dilution liquid chromatography-photospray ionization tandem mass spectrometry, a highly sensitive method which has been developed for the simultaneous determination of 11 steroids [Guo T, Taylor R L, Singh R J, Soldin S J. (2006)]. Briefly, 300 μl of acetonitrile containing the deuterated internal standards is added to the cell pellet containing 2×10⁸ cells, vortexed, and incubated for 30 min at RT. Two hundred μl of water is then added along with internal standards and the mixture is centrifuged to precipitate the proteins. After protein removal, 350 μl of supernatant is diluted with 1.4 ml of water and 1.5 ml of the resulting solution is injected into the LC-APPI-MS/MS (Applied Biosystems API-5000 triple quadrupole mass spectrometer equipped with an atmospheric pressure photoionization source).

### Submission of Microarray Data to GEO Repository

All microarray data will be reported according to the MIAME standards and submitted to the GEO repository for public access prior to publication of this manuscript.

### Results

### Differentially Expressed Genes Between Autistic Probands and Sibling Controls Implicate Steroid Biosynthetic Pathways

The log₂ ratios of relative gene expression from autistic and nonautistic siblings were analyzed by one-class SAM using both 100% and 70% data filtering, which requires that 100% or 70% of the samples, respectively, must have non-zero expression ratios in order to be considered for statistical analysis. Significant differentially expressed genes are presented in Tables 18 and 19 for each filtered dataset, which also report false discovery rates (FDR) to account for multiple testing for the respective data. Pathway Studio 5 and Ingenuity Pathway Analysis software was used to construct the major multigene interaction network which comprises genes (from the dataset with 100% data filtering) that were differentially expressed between normal and autistic siblings. Interestingly, this network includes cellular (apoptosis, differentiation, survival) [Hu V W, Frank B C, Heine S, Lee N H, Quackenbush J. (2006)] and disease processes (inflammation, digestion, epilepsy) that are often associated with ASD [Lathe R. (2006)]. Table 12 lists the top 5 (out of 56) high level functions that were identified by Ingenuity Pathway Analysis as being significantly overrepresented by differentially expressed genes in this dataset. Genes involved in the top 2 functions, endocrine system development and function and small molecule biochemistry, significantly implicate involvement of the steroid hormone biosynthetic pathway. This is further supported by Pathway Studio 5 analysis which shows that steroid hormones are an integral part of the network of common metabolic targets of this set of differentially expressed genes (data not shown). The top biological functions are recapitulated in the dataset of significant differentially expressed genes obtained with less restrictive 70% data filtering across all samples (Table 13). Significant neurologically relevant functions, such as morphology of Purkinje cells, development of cerebellum, differentiation, quantity, and morphology of central nervous system cells, are also revealed within this expanded dataset. A network showing the relationship between all of the genes in this table in addition to other genes is shown in FIG. 4. Interestingly, genes regulating inflammatory processes (eg., TNF, NFKB) lie at the core of this network, as was noted in our earlier study on monozygotic twins discordant in autism diagnosis [Hu V W, Frank B C, Heine S, Lee N H, Quackenbush J. (2006)]. Ingenuity Pathway Analysis lists the top 2 canonical pathways associated with this dataset of significantly differentially expressed genes as axon guidance (p=2.82E−02) and NRF2-mediated oxidative stress response (p=3.94E−02) in which the p values are derived from Fisher Exact tests of the probability that the dataset is not enriched for genes within a particular pathway.

Confirmation of Differentially Expressed Genes Related to Steroid Metabolism, Development, and Autism by qRT-PCR Analysis

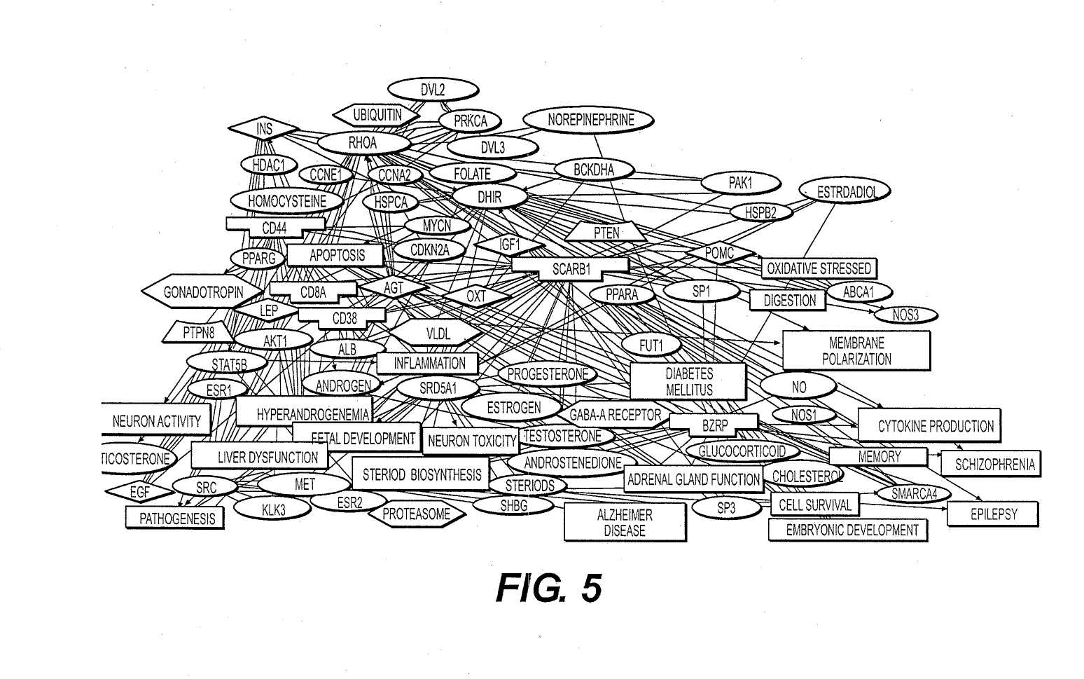

Quantitative RT-PCR (qRT-PCR) was used to confirm the differential expression of genes involved in cholesterol/steroid hormone metabolism as well as a selected number that are involved in development and/or associated with autism. FIG. 5 shows a gene network that is constructed from 11 of the qRT-PCR-confirmed genes, 5 of which are located in quantitative trait loci (QTL) based upon whole genome scans (Table 16). It is noteworthy that cholesterol as well as several steroid hormones, including testosterone, androstenedione, progesterone, estradiol, and estrogen, are among the common small molecule regulators of this network of genes suggesting the possibility of feedback regulation between these metabolites and genes involved in their production. Other small molecules within this network that may play a role in ASD are oxytocin (OXT), nitric oxide (NO), homocysteine (which is involved in transsulfuration reactions), folate (which is involved in development), norepinephrine, and the stress hormones glucocorticoid and corticosterone [Lathe R. (2006)]. Also interesting is the association of this gene network with inflammation, epilepsy, diabetes mellitus, digestive disorders, and hyperandrogenemia, all of which have been associated with ASD [Saemundsen E, Ludvigsson P, Hilmarsdottir I, Rafnsson V. (2007), Iafusco D, Vanelli M, Songini M, Chiari G, Cardella F, et al. (2006), Horvath K, Perman J A. (2002)]. Aside from the several novel candidate genes identified in this study, the network in FIG. 5 also includes 2 other genes, PAK1 and PTEN, which have been identified as candidate ASD genes in other studies [Baron C A, Liu S Y, Hicks C, Gregg J P. (2006)].

Steroid Profiling Reveals Elevated Testosterone Levels in LCL Extracts from Autistic Siblings

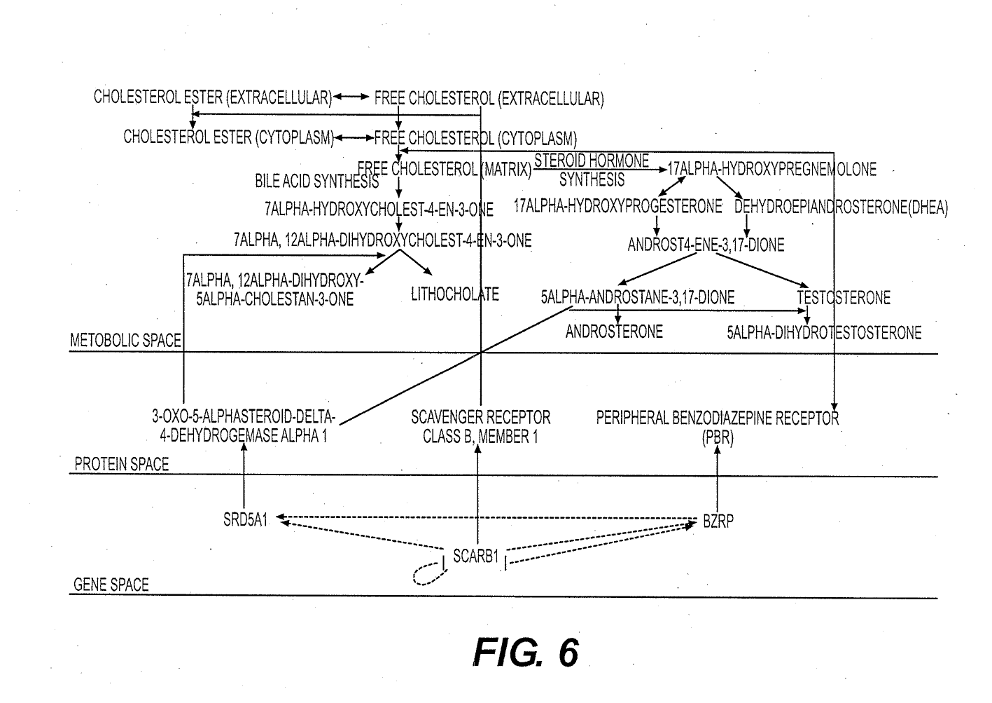

Based upon the qRT-PCR-confirmed differential expression of several of the genes involved in cholesterol metabolism and steroid hormone biosynthesis (SCARB1, BZRP, and SRD5A in particular), a multilevel biomolecular network was constructed representing the possible interactions and functions of the genes, gene products, and downstream metabolites (FIG. 6). From this bionetwork, it was postulated that elevations in some or all of these genes may lead to an increase in androgenic hormone biosynthesis. Indeed, Table 17 shows that testosterone was elevated in LCL extracts from 3 out of 3 autistic siblings relative to their respective non-autistic siblings.

### Discussion

It is becoming increasingly clear that although the neurological symptoms of ASD are the most striking among the behavioral and functional manifestations of affected individuals, there are many associated peripheral physiological symptoms that have often gone unnoticed/ignored and clinically unaddressed. These include gastrointestinal disorders experienced by many on the spectrum (estimated at 50%) as well as immune disorders which have long been described in the literature on ASD. The large-scale global gene expression profiling undertaken on LCL derived from peripheral blood lymphocytes of ASD probands and their respective siblings may therefore serve as a window to the underlying biochemical and signaling deficits that may be relevant to understanding the broader symptomatology of autism.

Overall, the study of autistic-nonautistic sib pairs in which the autistic sibling has been subtyped according to severity of language impairment on the basis of cluster analysis of scores from the ADIR diagnostic interview (unpublished data described in Example 1), reveals altered expression of several genes that participate in cholesterol metabolism and, in particular, androgen biosynthesis. This finding is supported by the pilot studies on the metabolites within this pathway which show elevated testosterone in the autistic sibling relative to his respective nearly age-matched normal sibling as well as by other studies in the literature which show elevated androgen levels in the serum of autistic individuals, including females [Geier D A, Geier M R. (2007), Knickmeyer R, Baron-Cohen S, Fane B A, Wheelwright S, Mathews G A, et al. (2006)]. The observation that at least 2 of the genes (SCARB1 and SRD5A1) that are involved in cholesterol import into the cell and testosterone metabolism exhibit increased expression in the autistic siblings offers a plausible explanation for elevated androgen levels in ASD.

The biological consequences of elevated testosterone on neurodevelopment and function are just beginning to be understood. While it has been known for more than 10 years that estrogens modulate synaptic plasticity in the hippocampus of female rats, it has only recently been shown that androgens likewise play a role in hippocampal synaptic plasticity, but in both males and females [MacLusky N J, Hajszan T, Prange-Kiel J, Leranth C. (2006)]. Furthermore, there is increasing evidence for the role of “neurosteroids” (which include DHEA and progesterone) in neurological functions, including rapid modulation of neurotransmitter receptors. In contrast to testosterone, DHEA which has been shown to be lowered in ASD [Strous R D, Golubchik P, Maayan R, Mozes T, Tuati-Wemer D, et al. (2005)], plays a neuroprotective role countering the effect of stress-inducing steroids [Kalimi M, Shafagoj Y, Loria R, Padgett D, Regelson W. (1994), Kimonides V G, Spillantini M G, Sofroniew M V, Fawcett J W, Herbert J. (1999)]. Interestingly, the levels of DHEA observed were lower in several of the autistic siblings relative to their respective nonautistic siblings (data not shown). Clearly, it will be important to further evaluate the levels of steroid hormones and precursor molecules in a broader sampling of individuals with ASD as well as to establish a correlation between these metabolite levels and aberrant expression of genes in this metabolic pathway.

Pathway analyses using Pathway Studio 5 also implicated involvement of female hormones in that the estrogens (including estradiol and ethinyl estradiol) were among the small molecule regulators of the differentially expressed genes. It is further noted that one of the differentially expressed genes listed in Table 12, SRD5A1, is involved in sex determination. Thus, the altered expression of genes involved in steroid hormone production and sexual dimorphism (eg., STAT5B), coupled with the differential impact of male and female steroid hormones on brain development in male vs. female animals may, in part, underlie the approximately 4:1 male to female ratio in ASD.

Bile acid synthesis might also be affected by some of the differentially expressed genes in ASD, particularly SCARB1 and BZRP, which respectively internalize cholesterol and move it into the mitochondria where it can be converted to bile acids by the appropriate enzymes. This suggests that dysregulation of genes in this pathway may also be responsible for the digestive and hepatic disorders associated with ASD. Indeed, in a separate case-control study of a large number of unrelated individuals (total of 116), hepatic cholestasis and fibrosis are strongly indicated on the basis of the gene expression profiles of the autistic probands versus unrelated controls. (unpublished data). Changes in metabolite profiles thus may be predicted and tested on the basis of a functional analysis of altered gene interactions that arise from increases or decreases in gene expression within a specific metabolic pathway. In turn, such an analysis may lead to a diagnostic screen for ASD based on metabolite profiling of serum or other easily accessible tissues (e.g., steroid hormone or bile acid assays).

Aside from genes involved in cholesterol metabolism and steroid hormone biosynthesis, the altered expression of several network-associated genes that are critical to developmental processes and/or associated with ASD (FIG. 5) was also confirmed. These include DVL2 and DVL3, both of which are involved in Wnt signaling, DHFR, a key enzyme involved in folate biosynthesis which is important for neural tube formation, RHOA, which is involved in Wnt signaling, axon guidance, cytoskeletal regulation, and dendrite branching, and STAT5B which is involved in the sexually dimorphic response to growth hormones [Tang Y, Lu A, Aronow B J, Sharp F R. (2001)]. Several of the confirmed differentially expressed genes in this network, CD38, CD44, and MET, have been previously associated with ASD through genetic analyses, thus suggesting a functional link between the genetic variations reported and transcriptional regulation, which has been previously reported for MET, a gene with known involvement in gastrointestinal and immune functions, both of which may be dysregulated in autism [Campbell D B, Sutcliffe J S, Ebert P J, Militemi R, Bravaccio C, et al. (2006)]. It is interesting to note that CD44 and MET, which are respectively up- and down-regulated in LCL, have also been reported to be similarly regulated in brain tissue from autistic individuals relative to controls [Campbell D B, D'Oronzio R, Garbett K, Ebert P J, Mimics K, et al. (2007)]. Moreover, additional recent studies provide support that blood expression profiling may be useful in identifying a subset of genes and/or more broadly ontological categories of genes undergoing dysregulation in the brain for a number of neurological disorders. Taken together, these studies provide strong support for the use of LCL as surrogate models to examine gene dysregulation in ASD. With respect to neurological function, MET has been shown to collaborate with CD44, its coreceptor, in synaptogenesis and axon myelination [Campbell D B, D'Oronzio R, Garbett K, Ebert P J, Mimics K, et al. (2007)], key processes associated with various candidate genes identified by genetic and gene expression analyses [Persico A M, Bourgeron T. (2006)]. CD38, on the other hand, is a gene that regulates the production of oxytocin, a peptide hormone that has been shown to be involved in social cognition and behavior [Jin D, Liu H-, Hirai H, Torashima T, Nagai T, et al. (2007)]. Finally, BZRP, a drug target of benzodiazepines which are prescribed for symptoms of anxiety often associated with ASD, is not only involved in cholesterol metabolism but also in embryogenesis [O'Hara M F, Nibbio B J, Craig R C, Nemeth K R, Charlap J H, et al. (2003)] and schizophrenia [Kurumaji A, Nomoto H, Yoshikawa T, Okubo Y, Toru M. (2000)]].

Pathway Studio 5 analyses of the targets and regulators of differentially expressed genes listed in Table 12 and Table 13 show the relationship between these genes and disorders that may be associated with autism, specifically, diabetes mellitus, digestive disorders, endocrine abnormality, epilepsy, hyperandrogenemia, hyperinsulinemina, immunodeficiency, inflammation, muscular dystrophy, neural tube malformation, and neuron toxicity (data not shown). It is suggested that dysregulation of genes in pathways associated with diabetes, insulin sensitivity, and/or inflammation as demonstrated in these studies may lead to the gastrointestinal disorders often manifested by individuals with ASD.

What is especially revealing from our studies is that, across all ASD samples relative to nonautistic sib controls, multiple genes are aberrantly expressed in canonical metabolic and signaling pathways (eg., steroidogenesis, axon guidance) critical to the development of autism. This suggests that in any given individual with ASD, these relevant pathways may be compromised by different genetic mutations and/or polymorphisms (i.e. SNPs, copy number variants) which, possibly in conjunction with currently unspecified environmental factors, may give rise to altered expression of different pathway-specific genes, ultimately resulting in a dysfunctional pathway which contributes to the phenotype of ASD. The genes, metabolites, and pathways identified in this study further suggest novel targets for therapeutics. Thus, gene expression profiling, which provides a global view of functional gene networks in the context of living cells from individuals with ASD, not only allows for the elucidation of compromised pathways but also provides a meaningful and complementary (with respect to genetics) approach towards understanding the complex biology of ASD.

TABLE 12
Biological functions identified by Ingenuity Pathway Analysis of genes within
the dataset of 100 significant genes identified by SAM analysis with 100% data
filtering. An expression cutoff of log₂(ratio) ≧±0.29 was applied before pathway
analysis.

| Category | Function Annotation | P-value | Molecules |
|---|---|---|---|
| Endocrine System Development | biosynthesis of androgen | 2.67E−06 | SCARB1, SRD5A1 |
| and Function | | | |
| Endocrine System Development | quantity of 4-androstene-3,17-dione | 3.23E−03 | SRD5A1 |
| and Function | | | |
| Small Molecule Biochemistry | breakdown of progesterone | 4.62E−04 | SRD5A1 |
| Small Molecule Biochemistry | endocytosis of cholesterol | 4.62E−04 | SCARB1 |
| Lipid Metabolism | Steroidogenesis | 1.61E−05 | SCARB1, SRD5A1 |
| Lipid Metabolism | absorption of triolein | 4.62E−04 | SCARB1 |
| Lipid Metabolism | Synthesis of ganglioside GM3 | 1.85E−03 | CD9 |
| Cell Morphology/Nervous System | morphology of neurons | 3.03E−05 | CD9, GATA3 |
| Development and Function | | | |
| Cell Morphology/Nervous System | morphology of serotonergic neurons | 4.62E−04 | GATA3 |
| Development and Function | | | |
| Cell Morphology/Nervous System | cell flattening of neuroglia, neurons | 4.62E−04 | CD9 |
| Development and Function | | | |

*Significance calculated for each function is an indicator of the likelihood of that function being associated with the dataset by random chance. The range of p-values was calculated using the right-tailed Fisher's Exact Test, which compares the number of user-specified genes to the total number of occurrences of these genes in the respective functional/pathway annotations stored in the Ingenuity Pathways Knowledge Base.

TABLE 13
Biological functions identified by Ingenuity Pathway Analysis of genes within
the dataset of 135 significant genes identified by SAM analysis with 70% data
filtering. An expression cutoff of log₂(ratio) ≧±0.29 was applied before pathway
analysis.

| Category | Function Annotation | P-value | Molecules |
|---|---|---|---|
| Endocrine System | biosynthesis of androgen | 4.89E−05 | SCARB1, |
| Development and Function | | | SRD5A1 |
| Endocrine System | proliferation of pancreatic duct | 3.69E−03 | CXCR4 |
| Development and Function | cells | | |
| Endocrine System | quantity of 4-androstene-3,17- | 1.29E−02 | SRD5A1 |
| Development and Function | dione | | |
| Small Molecule Biochemistry | endocytosis of cholesterol | 1.85E−03 | SCARB1 |
| Small Molecule Biochemistry | breakdown of progesterone | 1.85E−03 | SRD5A1 |
| Small Molecule Biochemistry | biosynthesis of norepinephrine | 5.53E−03 | GATA3 |
| Small Molecule Biochemistry | synthesis of ganglioside GM3 | 7.37E−03 | CD9 |
| Small Molecule Biochemistry | synthesis of norepinephrine | 1.10E−02 | GATA3 |
| Small Molecule Biochemistry | uptake of taurocholic acid | 1.83E−02 | PRKCZ |
| Nervous System Development | morphology of neurons | 5.49E−04 | CD9, GATA3 |
| and Function | | | |
| Nervous System Development | morphology of Purkinje cells | 1.85E−03 | ATP2B2 |
| and Function | | | |
| Nervous System Development | morphology of serotonergic | 1.85E−03 | GATA3 |
| and Function | neurons | | |
| Nervous System Development | fusion of vagus cranial nerve | 1.85E−03 | LMO4 |
| and Function | ganglion | | |
| Nervous System Development | polarization of astrocytes | 1.85E−03 | PRKCZ |
| and Function | | | |
| Nervous System Development | Development of cerebellum | 1.98E−03 | ATP2B2, CXCR4 |
| and Function | | | |
| Nervous System Development | branching of sympathetic | 3.69E−03 | LIFR |
| and Function | neuron | | |
| Nervous System Development | Differentiation/quantity of | 5.43E−03 | ATP2B2, LIFR |
| and Function | central nervous system cells | | |
| Nervous System Development | morphology of central nervous | 5.53E−03 | ATRN |
| and Function | system | | |
| Nervous System Development | Development of Purkinje cells | 1.10E−02 | CXCR4 |
| and Function | | | |
| Nervous System Development | migration of motor neurons | 1.83E−02 | GATA3 |
| and Function | | | |
| Nervous System Development | biogenesis of synapse | 2.74E−02 | ATP2B2 |
| and Function | | | |
| Nervous System Development | guidance of motor axons | 2.74E−02 | CXCR4 |
| and Function | | | |

*Significance calculated for each function is an indicator of the likelihood of that function being associated with the dataset by random chance. The range of p-values was calculated using the right-tailed Fisher's Exact Test, which compares the number of user-specified genes to the total number of occurrences of these genes in the respective functional/pathway annotations stored in the Ingenuity Pathways Knowledge Base.

TABLE 16
Quantitative trait loci (QTL) associated with RT-qPCR-confirmed genes.

| | Gene | Mean | | | |
|---|---|---|---|---|---|
| Genbank # | symbol | log2(ratio)* | Chromosomal location | QTL | Ref. |
| AA455945 | BZRP | −0.5 | chr22: 41,888,752-41,889,192 | | |
| R00276 | CD38 | 0.26 | chr4: 15,459,258-15,459,578 | 3,639,365-17,076,888 | 92 |
| H03494 | CD44 | 0.49 | chr11: 35,183,785-35,184,167 | 30,990,001-43,410,000 | 43 |
| N52980 | DHFR | 0.38 | chr5: 79,859,237-80,059,364 | | |
| AA812964 | DVL2 | 0.87 | chr17: 7,069,385-7,069,663 | 3,613,299-36,248,135 | 93 |
| W84790 | DVL3 | −1.32 | chr3: 185,357,257-185,374,008 | | |
| AA017355 | MET | −1.81 | chr7: 116,099,695-116,225,632 | 115,682,101-116,992,078 | 94 |
| AA676955 | RHOA | −0.88 | chr3: 49,371,582-49,371,973 | | |
| AA443899 | SCARB1 | 0.62 | chr12: 123,828,127-123,828,543 | | |
| R36874 | SRD5A1 | 0.59 | chr5: 6,622,352-6,822,675 | 3,174,219-7,711,583 | 95 |
| AA282023 | STAT5B | −1.23 | chr17: 37,621,607-37,623,875 | | |

Each assay was run in duplicate (and normalized against an 18S rRNA standard curve for each sample) or in triplicate using the comparative threshold cycle method.
*Mean log₂(ratio) of gene expression in LCL from autistic vs. unaffected sibling.

TABLE 17
Concentration of testosterone in LCL extracts from 3 pairs of autistic-
nonautistic siblings as determined by HPLC-MS/MS analyses.

| Sample | Age | Status | Testosterone (ng/dL) | Ratio (autistic/normal) |
|---|---|---|---|---|
| HI0366 | 18 | autistic | 241 | 1.14 |
| HI0365 | 20 | normal | 212 | |
| HI0355 | 12 | autistic | 218 | ≧218 |
| HI0354 | 14 | normal | <1 | |
| HI2769 | 10 | autistic | 251 | 1.22 |
| HI2772 | 13 | normal | 206 | |

TABLE 18
Significant differentially expressed genes from SAM analysis of
microarray data from sib pairs with data filter set at 100%, which
requires that 100% of the samples must have non-zero expression
ratios in order to be considered for statistical analyses. FDR ≦19.2%.
Genes shown in this table have a mean log₂(ratio) of ≧±0.29
in LCL from autistic vs. unaffected sibling.

| | Gene | Mean log2 |
|---|---|---|
| Genbank# | symbol | (ratio)* |
| H10192 | LIFR | 0.51 |
| AA926764 | VPREB3 | 0.50 |
| T62491 | CXCR4 | 0.47 |
| H72122 | unknown | 0.42 |
| N73575 | TRIM25 | 0.41 |
| H69786 | NFKBIZ | 0.38 |
| AA025380 | GATA3 | 0.38 |
| AA410291 | FGD6 | 0.36 |
| H90147 | BCL7A | 0.36 |
| AA412053 | CD9 | 0.35 |
| AI061421 | unknown | 0.35 |
| AA625666 | LITAF | 0.34 |
| AA455945 | BZRP | 0.33 |
| AA292086 | FAM102A | 0.32 |
| AA705886 | MXI1 | 0.31 |
| N69689 | RAB1A | 0.30 |
| AA443899 | SCARB1 | 0.29 |
| R36874 | SRD5A1 | 0.29 |
| AA256157 | C13ORF25 | 0.29 |
| AA449750 | CECR5 | 0.29 |
| AI198213 | RNU12P | −0.55 |
| AA939238 | unknown | −0.65 |
| N51674 | COL24A1 | −0.65 |

TABLE 19
Significant differentially expressed genes from
SAM analysis of microarray data from sib pairs with data
filter set at 70%, which requires that 70% of the samples
must have non-zero expression ratios in
order to be considered for statistical analyses.
There were 135 significant genes with a FDR ≦
13.5% and 264 genes with a FDR ≦ 15.9%.
Genes shown in this table have a mean log₂(ratio)
of ≧±0.29 in LCL from autistic vs. unaffected sibling.

| Genbank | Gene | Log2 | % |
|---|---|---|---|
| # | symbol | (ratio) | FDR |
| AA148736 | P15RS | 0.87 | 13.5 |
| AA453783 | MAL2 | 0.72 | 15.9 |
| R92176 | AGXT2 | 0.72 | 15.9 |
| AA426307 | GNAQ | 0.61 | 15.9 |
| AA894442 | SIX4 | 0.57 | 15.9 |
| AA857851 | CUL5 | 0.55 | 15.9 |
| AA609471 | IER5L | 0.54 | 13.5 |
| AA953747 | PLS3 | 0.51 | 15.9 |
| N25987 | DIRC2 | 0.51 | 15.9 |
| H10192 | LIFR | 0.51 | 13.5 |
| AA926764 | VPREB3 | 0.50 | 15.9 |
| AA907419 | FOXF1 | 0.48 | 15.9 |
| T62491 | CXCR4 | 0.47 | 13.5 |
| AA461118 | DMD | 0.43 | 15.9 |
| AA425373 | CAMK2N1 | 0.42 | 15.9 |
| AI091450 | SYTL3 | 0.42 | 15.9 |
| H72122 | unknown | 0.42 | 15.9 |
| R28287 | unknown | 0.41 | 13.5 |
| N73575 | TRIM25 | 0.41 | 13.5 |
| H69786 | NFKBIZ | 0.38 | 13.5 |
| AA025380 | GATA3 | 0.38 | 13.5 |
| AA007370 | HKR1 | 0.37 | 13.5 |
| R83847 | LOC388335 | 0.37 | 13.5 |
| R97066 | TAL1 | 0.36 | 15.9 |
| AA410291 | C2ORF17 | 0.36 | 13.5 |
| H90147 | BCL7A | 0.36 | 13.5 |
| AA412053 | CD9 | 0.35 | 13.5 |
| AI061421 | unknown | 0.35 | 13.5 |
| AI283902 | HIST1H1A | 0.34 | 13.5 |
| AA007634 | SNX24 | 0.34 | 15.9 |
| N54162 | CCNE2 | 0.34 | 15.9 |
| AA902823 | SPATA16 | 0.34 | 15.9 |
| AA884151 | GPR175 | 0.34 | 13.5 |
| T97917 | unknown | 0.34 | 15.9 |
| AA625666 | LITAF | 0.34 | 13.5 |
| AA487739 | GOT2 | 0.34 | 13.5 |
| R32996 | unknown | 0.33 | 13.5 |
| AI131501 | unknown | 0.33 | 15.9 |
| AA458486 | COMMD4 | 0.33 | 13.5 |
| AA455945 | BZRP | 0.33 | 15.9 |
| H15535 | PDE4DIP | 0.33 | 13.5 |
| R26792 | GCA | 0.32 | 15.9 |
| AA292086 | FAM102A | 0.32 | 15.9 |
| AA973009 | C16ORF44 | 0.32 | 13.5 |
| AI159943 | PLAGL1 | 0.32 | 15.9 |
| AA424887 | SMG6 | 0.31 | 15.9 |
| H20826 | unknown | 0.31 | 13.5 |
| AA932364 | C18ORF14 | 0.31 | 13.5 |
| R07295 | SOAT1 | 0.31 | 15.9 |
| AA984306 | HMBOX1 | 0.30 | 13.5 |
| AI217709 | unknown | 0.30 | 13.5 |
| AA487527 | DTX4 | 0.30 | 15.9 |
| N69689 | RAB1A | 0.30 | 15.9 |
| AA443899 | SCARB1 | 0.29 | 13.5 |
| R36874 | SRD5A1 | 0.29 | 13.5 |
| R72661 | FLJ23861 | 0.29 | 15.9 |
| AA429572 | WASF2 | 0.29 | 13.5 |
| R12679 | unknown | 0.29 | 13.5 |
| AA256157 | C13ORF25 | 0.29 | 13.5 |
| AA457153 | ZNF282 | 0.29 | 15.9 |
| H55784 | FOXP1 | 0.29 | 15.9 |
| AA037619 | LOC146346 | 0.29 | 13.5 |
| AI680609 | DIP | 0.29 | 15.9 |
| AA449750 | CECR5 | 0.29 | 13.5 |
| AI221690 | PRKCZ | −0.30 | 13.5 |
| AA424531 | LOC133993 | −0.31 | 13.5 |
| AI141767 | unknown | −0.32 | 13.5 |
| N80619 | ATRN | −0.32 | 13.5 |
| AA115054 | KCTD12 | −0.32 | 13.5 |
| AA644559 | LMO4 | −0.33 | 13.5 |
| AI421603 | ATP2B2 | −0.34 | 13.5 |
| AI291307 | SVIL | −0.36 | 13.5 |
| AI268273 | MAP3K5 | −0.42 | 15.9 |
| AI291693 | C21ORF34 | −0.48 | 13.5 |
| AI122714 | unknown | −0.50 | 13.5 |
| AI198213 | RNU12P | −0.55 | 13.5 |
| AA044664 | SCN5A | −0.61 | 13.5 |
| AA939238 | unknown | −0.65 | 13.5 |
| N51674 | COL24A1 | −0.65 | 13.5 |

TABLE 20
Primer sequences for qRT-PCR analyses (“FORWARD
SEQUENCE” primers disclosed as SEQ ID NOS 23-33
and “REVERSE SEQUENCE” primers disclosed as
SEQ ID NOS 34-44, all respectively,
in order of appearance.)

| | FORWARD SEQUENCE | REVERSE SEQUENCE |
|---|---|---|
| GENE | (5′ → 3′) | (5′ → 3′) |
| CD38 | TGG GAA CTC AGA | TAG CCT AGC AGC |
| | CCG TAC CT | GTG TCC TC |
| CD44 | ATC ACC GAC AGC | GGT TGT GTT TGC |
| | ACA GAC AG | TCC ACC TT |
| DHFR | CTC AAG GAA CCT | GCC ACC AAC TAT |
| | CCA CCA GG | CCA GAC CA |
| DVL2 | GTA TCC TGG CTG | TGG CAA AGG AGG |
| | GTG TCC TC | TAA AGG TG |
| DVL3 | GAT TTC GGA GTG | CAG CTC CGA TGG |
| | GTG AAG GA | GTT ATC AG |
| MET | AAG AGG GCA TTT | CTC GGT CAG AAA |
| | TGG TTG TG | TTG GGA AA |
| RHOA | CCA TCG ACA GCC | GCC TTG TGT GCT |
| | CTG ATA GT | CAT CAT TC |
| SCARB1 | CCC ATC CTC ACT | GCT CAG CTA CAG |
| | TCC TCA AC | TTT CAC AG |
| SRD5A1 | GGG TAA CAG ATC | CAA ATA AGC CTC |
| | CCC GTT TT | CCC TTG GT |
| STAT5B | TTG ACG GTG TGA | AGT AGG TCA TGG |
| | TGG AAG TG | GCC TGT TG |
| TSPO | GCC CGA CAA ATG | CCA CGC CAG CCA |
| | GGC TGG G | TGG TTG T |

### Example 4

### Development of a Predictive Gene Classifier for Autism Spectrum Disorders Based Upon Differential Gene Expression Profiles

This Example demonstrates that several phenotypic variants of idiopathic autism can be distinguished from nonautistic controls on the basis of differential gene expression of limited sets of genes in lymphoblastoid cell lines (LCL) from the respective individuals with a predicted classification accuracy of up to 98%. The data suggests that such sets of genes may be useful biomarkers for diagnosis of idiopathic autism.

### Materials and Methods

Analysis of Data from ADIR Questionnaires to Identify Phenotypic Subgroups

ADIR score sheets were downloaded for 1954 individuals with autism from the Autism Genetic Research Exchange (AGRE) phenotype database. A total of 123 items that were identical or comparable on both 1995 and 2003 versions of the ADIR were included. “Current” and “ever” scores were used for most of these items. Only items scored numerically (0=normal; 3=most severe) were analyzed. A score of 8 for items in the spoken language subgroup indicated that the items were not applicable because of insufficient language and was replaced with a rating of 3. Scores of 8 or 9 for other items (excluding those from the spoken language subgroup), which indicated the item was not asked or not applicable, were replaced with blanks to reflect that no information was available for that item. A score of 1 or 2 on item 19 (LEVELL) indicated an overall language deficit and, as a result, scores for items 20-28 were assigned a score of 3 to reflect impaired language skills, as previously done by Tadevosyan-Leyfer, et al. (2003). Items with scores of 4 in the savant skill subgroup, which meant that the individual possessed an isolated though meaningful skill/knowledge above that of his general functional level or the population norm, were replaced with 3 to maintain consistency of the 0-3 scale across all items. Scores of 7 for some items were changed to a score between 0 and 3 depending on the nature of the question and how it reflected severity with respect to that specific item. A score of −1 indicated missing data (according to AGRE) and was replaced with a blank.

Data on ADIR score sheets for 1954 individuals were loaded into MeV (Saeed A I, Sharov V, White J, Li J, Liang W, et al. (2003)), a software program created by John Quackenbush and colleagues to analyze microarray gene expression data. Each individual was represented by a horizontal row in the data matrix while ADIR items were represented by vertical columns. Multiple clustering analyses were employed to subgroup individuals on the basis of ADIR item scores and included principal components analysis (PCA), hierarchical clustering (HCL), and k-means clustering (KMC), which is a “supervised” clustering method. A fitness of merit (FOM) analysis was also conducted to estimate the optimal number of clusters, while correspondence analysis (COA) was used to visualize the association of specific items with clusters of individuals. A description of each of these analytical methods is summarized by Saeed et al.

### Selection of Samples for Large-Scale Gene Expression Analyses

Lymphoblastoid cell lines (LCL) for DNA microarray analyses were selected on the basis of phenotypic clustering of autistic individuals using the methods described above. As described in the results, the application of multiple clustering algorithms to the selected ADIR items from scoresheets of 1954 individuals resulted in 4 reasonably distinct phenotypic subgroups. Samples were selected from 3 of the 4 groups for gene expression analyses. These groups included those with severe language impairment, those with milder symptoms across all domains, and those defined by presence of notable savant skills. Additional selection criteria were applied to exclude all female subjects, individuals with cognitive impairment (Raven's scores<70), those with known genetic or chromosomal abnormalities (e.g., Fragile X, Retts, tuberous sclerosis, chromosome 15q11-q13 duplication), those born prematurely (<35 weeks gestation), and those with diagnosed comorbid psychiatric disorders (e.g., bipolar disorder, obsessive compulsive disorder, severe anxiety). In addition, a score<80 on the Peabody Picture Vocabulary. Test (PPVT) was used to confirm language deficits for those in the group identified by cluster analysis as having severe language impairment. In this study, 26-31 cell lines were obtained for each of 3 selected study groups, along with 29 cell lines from “control” individuals who were nonautistic siblings of those with autism, matched roughly in age to the individuals with autism.

### Cell Culture

The LCL were cultured as previously described (Hu V W, Frank B C, Heine S, Lee N H, Quackenbush J. (2006)) according to the protocol specified by the Rutgers University Cell and DNA Repository, which maintains the Autism Genetic Research Exchange (AGRE) collection of biological materials from autistic individuals and relatives. Briefly, cells are cultured in RPMI 1640 supplemented with 15% fetal bovine serum, and 1% penicillin/streptomycin. Cultures are split 1:2 every 3-4 days and cells are typically harvested for RNA isolation 3 days after a split while the cultures are in logarithmic growth phase.

### Gene Expression Analyses on Spotted DNA Microarrays

Gene expression profiling is accomplished using TIGR 40K human arrays as previously described (Hu V W, Frank B C, Heine S, Lee N H, Quackenbush J. (2006)). Total RNA was isolated from LCL using the TRIzol (Invitrogen) isolation method according to the manufacturer's protocols, and cDNA was synthesized, labeled, and hybridized to the microarrays as described in our earlier study, with the exception that cDNA from each sample was labeled with Cy-3 dye and hybridized against Cy-5 labeled reference cDNA prepared from Universal human RNA (Stratagene). This “reference” design allows the flexibility to perform different comparisons among the samples since all expression values are against a common reference. After hybridization, washing of the arrays, and laser scanning to elicit dye intensities for each element on the array, the intensity data was normalized and filtered using Midas and analyzed using MeV, which are open-access software programs for DNA microarray analyses. All analyses were performed with a 100% data filter which means that each gene included in the analyses must have an expression value in 100% of the samples. Unpaired t-tests were used to obtain significant differentially expressed genes which were then subjected to class prediction and validation methods to identify the most robust genes for predicting cases and controls.

### Class Prediction and Validation Methods

Two supervised learning methods were employed to identify highly predictive genes for ASD and these methods were applied to discriminate each of the members of the ASD subgroups from controls as well as to discriminate members of the combined ASD groups and controls. Significant differentially expressed genes derived from the t-test analyses were analyzed using USC with 10-fold cross-validation to identify a limited set of genes which were further tested by SVM analyses with 10-fold cross-validation to determine the accuracy of correctly assigning samples to cases and controls.

### Results and Discussion

A major goal of this study was to identify groups of genes that may be used to discriminate autistic from nonautistic individuals, and to ultimately develop a diagnostic screen for autism. Towards this goal, DNA microarray analyses were performed to obtain the gene expression profiles of lymphoblastoid cell lines (LCL) of 87 autistic male individuals who were divided into 3 phenotypic subgroups based on cluster analyses of scores on the Autism Diagnostic Interview-Revised questionnaire (Hu and Steinberg, manuscript submitted). These profiles were compared against that obtained from LCL of 29 nonautistic male control subjects. Here, gene classification and validation software were utilized to identify sets of genes that have a high statistical probability of predicting cases and controls.

### Identification of Classifier Genes for 3 Phenotypic Variants of ASD

Gene expression data obtained using a 40K TIGR human cDNA array with 39,936 probe elements was subjected to a 100% data filter that eliminated genes that were absent in any one of the samples under study (manuscript submitted). Unpaired t-tests were performed on the filtered data from each of the ASD subgroups and from the nonautistic controls to identify significantly differentiated genes (p≦0.01) between each subgroup and controls. Two different supervised learning methods were used to select genes for our predictive models. Uncorrelated Shrunken Centroids (USC) as implemented in MeV 3.1 software was first used to select the most robust classifier genes from the lists of significant genes, using training and test sets coupled with 10-fold cross-validation methods (Tables 21-23). The limited sets of classifier genes from the USC analyses were then entered into the support vector machine (SVM) software program (in MeV 3.1), again with 10-fold cross-validation to test the gene classifier for each of the phenotypic variants. As shown in Table 24, gene classifiers based upon the gene expression data can discriminate between each of the ASD phenotypic variants with an overall accuracy of ˜98%, with the number and identity of classifier genes dependent on the phenotype. In addition to the method of identifying highly predictive genes described above, a t-test was also employed with an adjusted Bonferroni correction for multiple testing to identify significantly differentiated genes between the most severe ASD group and controls. The resultant set of 24 genes (Table 25) also could correctly distinguish ASD from controls with 98% accuracy as indicated by SVM analysis. If all autistic samples are combined and tested against the nonautistic controls, the accuracy of correct assignment to case or control groups is 93%, based upon 88 differentially expressed genes (Table 26).

This study is the first to report classification methods for idiopathic autism based upon gene expression profiling. Furthermore, the profiles are of cultured cells derived from peripheral tissue (blood) demonstrating the potential for translation to clinical testing. These predictive gene classifiers are currently being evaluated using new LCL samples and by different analytical methods, such as the microtiter Array Plate-based-quantitative nuclease protection assay (qNPA) which is more amenable to direct testing of clinical (blood) samples.

### Conclusion

The Example demonstrated that cases of idiopathic autism can be segregated from nonautistic controls with a high degree of accuracy based upon limited panels of predictive genes, which are specific for different phenotypic variants of ASD. These gene panels should be further investigated as potential biomarker screens for idiopathic autism. Early identification of autism based on objective gene screening is a major first step towards early intervention and effective treatment of affected individuals.

TABLE 21
Classifier genes which distinguish ASD with severe language impairment
from controls based upon combined USC and SVM analyses

| Genbank # | GeneSymbol |
|---|---|
| AA455126 | ATP5G2 |
| N51323 | BTG1 |
| AA455945 | BZRP, TSPO |
| N57483 | C21ORF63 |
| AA262235 | DDX26 |
| R54846 | FGFR1 |
| AA291183 | FLJ11021 |
| AA045665 | GLT28D1 |
| T68440 | GNE |
| H99811 | HNRPA3 |
| AA436187 | ITGAM |
| AA779937 | KIAA1706 |
| N26163 | LOC389831 |
| AI301365 | LOC389833 |
| AA932558 | MRPL14 |
| AA598632 | PPP1R9B, NEU |
| AA862434 | PSMB9 |
| H99843 | QPRT |
| AI689992 | RPS12 |
| N53133 | STRBP |
| T64881 | UBAP1 |
| AA156342 | UPF1 |
| AA205598 | WDR72 |
| N72256 | ZADH2 |
| AA256471 | ZNF189 |
| AI187812 | Unknown |
| AA013481 | Unknown |
| R26811 | Unknown |
| R26614 | Unknown |

TABLE 22
Classifier genes which distinguish mild ASD individuals from controls
based upon combined USC and SVM analyses

| | Gene |
|---|---|
| Genbank # | Symbol |
| AA458959 | ARID1A |
| AA132226 | CBX3 |
| AA195021 | CCDC47 |
| AA463411 | CSPG6 |
| AI241419 | DYSF |
| H19429 | ERO1LB |
| AA465236 | FOXO3A |
| H29301 | LMTK2 |
| AA482328 | MARCKS |
| AA164630 | MINA |
| H18953 | MLR2 |
| AA101630 | MYST3 |
| AA489785 | NCOA1 |
| AA176957 | NEB |
| R07319 | PHC3 |
| H65596 | SAP18 |
| AA136692 | TLE3 |
| AA703625 | TMEM16F |
| H17635 | TNKS2 |
| AA897665 | TRIO |
| T57841 | UFD1L |
| AA284243 | ZBTB4 |
| R39217 | ZNF447 |
| H14231 | unknown |
| W52000 | unknown |
| H15704 | unknown |

TABLE 23
Classifier genes which distinguish ASD individuals with notable savant
skills from controls based upon combined USC and SVM analyses

| Genbank # | Gene Symbol |
|---|---|
| H29771 | ATF6 |
| AA700707 | ATP11B |
| AA705040 | BGLAP |
| AA906454 | C14ORF108 |
| R55017 | C1ORF52 |
| AA490235 | EGLN2 |
| AA436405 | IGSF9 |
| H39221 | KLHL17 |
| H18949 | PAQR8 |
| R08116 | PARD3 |
| AA133281 | RNF36 |
| AA702428 | RNPC2 |
| R39039 | RUFY2 |
| T54320 | TOR1A |
| AA232979 | ZFR |
| T69553 | unknown |
| AI191562 | unknown |
| R06119 | unknown |

TABLE 24
Summary of class predictor accuracies based upon USC and SVM
analyses for respective sets of genes discriminating all ASD individuals
(A) from controls (C) as well as individuals from each ASD phenotype
tested (L, M, or S) and controls.

| | Accuracy of class predictor | | |
|---|---|---|---|
| | | [correct | |
| Comparison | USC→SVM | assignment] | (# genes) |
| A vs C | 93.9% | [109/116] | (88) |
| L vs C | 98.3% | [59/60] | (29) |
| M vs C | 98.2% | [54/55] | (26) |
| S vs C | 98% | [49/50] | (18) |

TABLE 25
Classifier genes which distinguish ASD with severe language impairment
from controls based upon an unpaired t-test with adjusted Bonferroni
correction for multiple testing as indicated by SVM analysis with
10-fold cross-validation

| GB# | Gene Symbol |
|---|---|
| AA910213 | ALS2CL |
| H72520 | BRD2 |
| N68510 | BRD3 |
| AA455945 | BZRP, TSPO |
| AI733697 | C12ORF30 |
| T50828 | CASP7 |
| AA262235 | DDX26 |
| AI050014 | DDX31 |
| AA291183 | FLJ11021 |
| AA633847 | FUSIP1 |
| T55592 | HNRPD |
| AA609738 | HNRPD |
| AA436187 | ITGAM |
| AI492016 | JAK1 |
| T68845 | MYLE, DEXI |
| R06605 | PTPN1 |
| AI583623 | SFRS10 |
| AA443300 | SMCP-2, MMP15 |
| W91960 | SSBP3 |
| AA133566 | TFIIE-beta, |
| | GTF2E2 |
| AA663944 | TRIM3 |
| AA676649 | TSHZ2 |
| AA156342 | UPF1 |
| AI187812 | qe10h08.x1 |

TABLE 26
Classifier genes which distinguish combined ASD individuals from
controls based upon combined USC and SVM analyses

| Genbank # | GENE_SYMBOL |
|---|---|
| AA625667 | ANKRD13C |
| R92545 | ARL15 |
| AA702802 | AZU1 |
| AA402984 | B3GALT6 |
| W38022 | BSPRY |
| AA455945 | BZRP |
| AI733697 | C12ORF30 |
| R55017 | C1ORF52 |
| N57483 | C21ORF63 |
| AA181868 | C9ORF5 |
| N94234 | CBL |
| AA418546 | CD109 |
| AA625651 | COPS2 |
| AA994790 | CSNK2B |
| AA262235 | DDX26 |
| AA999990 | EIF4A2 |
| N94428 | EP300 |
| AA598956 | ETNK1 |
| R54846 | FGFR1 |
| AA490046 | FIBP |
| AA521371 | FLJ22555 |
| AA021202 | FLJ32130 |
| AA400144 | GGN |
| AA281548 | HCCS |
| AA634028 | HLA-DPA1 |
| AA479962 | HNRPC |
| W31479 | HOMER1 |
| AA433916 | HSPA4 |
| H59805 | IGF2BP1 |
| T52830 | IGFBP5 |
| N27159 | INHBA |
| AA436187 | ITGAM |
| AA448164 | KBTBD2 |
| H85885 | KIAA0999 |
| AA448855 | LMOD3 |
| AA398321 | LOC133993 |
| AA426066 | LOC152217 |
| AA482328 | MARCKS |
| AA465188 | MCFP |
| AA101822 | MESDC1 |
| AA598949 | MFAP3 |
| AA476584 | MGC12966 |
| AA443300 | MMP15 |
| T72581 | MMP9 |
| N77198 | Unknown |
| H21071 | NAIP |
| AA167269 | NAP1L1 |
| N63178 | NHLRC2 |
| AA634267 | NPC1 |
| H14604 | PANK1 |
| AA625964 | PCGF3 |
| N40951 | PDPK1 |
| T95053 | PER1 |
| R16146 | PFKFB2 |
| AA496455 | PGM3 |
| AA976909 | PHF3 |
| AA428195 | PTPN2 |
| AA127069 | RIS1 |
| R53542 | SDC3 |
| AA608548 | SET |
| AA428181 | SPIN |
| N53133 | STRBP |
| AA479252 | TM9SF2 |
| AA159669 | TMEM49 |
| T54320 | TOR1A |
| AA156342 | UPF1 |
| T71990 | WBP2 |
| AA417318 | WDR33 |
| H79705 | WDR40A |
| AA495944 | WDR68 |
| AA598802 | WTAP |
| AA452107 | ZNF207 |
| AA598505 | ZNF434 |
| AA421352 | Unknown |
| AA621339 | Unknown |
| AA664228 | Unknown |
| AI187812 | Unknown |
| AI337100 | Unknown |
| H09082 | Unknown |
| N23009 | Unknown |
| N71463 | Unknown |
| R20640 | Unknown |
| R26614 | Unknown |
| R26811 | Unknown |
| R38613 | Unknown |
| R39258 | Unknown |
| R44214 | Unknown |
| T95670 | Unknown |

TABLE 27
Significant differentially expressed genes with log₂(ratio) ≧±0.3
from a 2-class SAM analysis of DNA microarray data from autistic
samples (31 cases) with severe language impairment (L subgroup) vs.
controls (29 subjects). FDR ≦5%

| Genbank# | GENE_SYMBOL | log2(L/C) |
|---|---|---|
| W69791 | ADCY1 | 1.13 |
| H19227 | ST3GAL6 | 1.12 |
| AA676466 | ASS | 1.05 |
| W69399 | H1F0 | 1.05 |
| H57830 | H1F0 | 1.04 |
| R38090 | C11ORF41 | 1.02 |
| AA884052 | ST3GAL6 | 1.00 |
| R33103 | SPG20 | 0.91 |
| R63543 | NGFRAP1 | 0.88 |
| AA448157 | CYP1B1 | 0.86 |
| AA461071 | SLC23A2 | 0.85 |
| AA865590 | BCAT1 | 0.84 |
| AA418748 | LOC389831 | 0.82 |
| AA676405 | ASS | 0.81 |
| AA418546 | CD109 | 0.81 |
| AI371096 | DAPK1 | 0.78 |
| AA455945 | BZRP | 0.77 |
| AA256386 | STARD13 | 0.76 |
| R62780 | PVRL3 | 0.76 |
| AI278292 | CD109 | 0.75 |
| AA150422 | CYBRD1 | 0.74 |
| AA911832 | GPRC5A | 0.73 |
| N69689 | RAB1A | 0.73 |
| AA443116 | RAI17 | 0.71 |
| AA702797 | KLHL6 | 0.71 |
| AA497040 | STC2 | 0.71 |
| AA598781 | IRF2BP2 | 0.70 |
| AA425947 | DKK3 | 0.70 |
| R56082 | SV2B | 0.70 |
| R54846 | FGFR1 | 0.70 |
| H98215 | CAMK2N1 | 0.69 |
| R55334 | KIAA1922 | 0.68 |
| R31938 | OPRK1 | 0.68 |
| AA634063 | TMEM22 | 0.67 |
| AI733650 | ZDHHC1 | 0.67 |
| AI275120 | LOC130576 | 0.66 |
| AA148524 | DDR2 | 0.66 |
| AA455350 | DFNA5 | 0.64 |
| H17493 | MAP1B | 0.64 |
| AA992985 | FLJ12825 | 0.64 |
| AA496253 | ATF5 | 0.64 |
| H68848 | APOH | 0.63 |
| AA464180 | BEX2 | 0.63 |
| H79047 | IGFBP2 | 0.63 |
| AA055076 | NR2F2 | 0.62 |
| AA701502 | PDGFA | 0.62 |
| AA284668 | PLAU | 0.62 |
| N26163 | LOC389831 | 0.62 |
| AA451886 | CYP1B1 | 0.61 |
| AA938573 | TBXAS1 | 0.61 |
| N32295 | ST3GAL6 | 0.60 |
| AA985354 | CDR1 | 0.60 |
| N29393 | UBXD7 | 0.60 |
| AA857705 | LOC401131 | 0.60 |
| T97599 | DTX1 | 0.60 |
| AA292086 | FAM102A | 0.60 |
| N73551 | INPP5F | 0.59 |
| H15040 | BCAS1 | 0.59 |
| R96522 | PSG1 | 0.59 |
| R59992 | ADCY1 | 0.58 |
| AA025275 | DAPK1 | 0.58 |
| AI341427 | BCAT1 | 0.58 |
| W74070 | ABCA8 | 0.58 |
| AI141972 | MARCH6 | 0.58 |
| AI018016 | LOC401089 | 0.58 |
| AI733556 | LOC401131 | 0.57 |
| AI160644 | NA | 0.57 |
| AI302412 | DCBLD2 | 0.57 |
| AA205072 | RP4-691N24.1 | 0.57 |
| T99645 | KCTD5 | 0.56 |
| W19228 | NA | 0.56 |
| N63635 | PIM1 | 0.56 |
| H18646 | ZNF532 | 0.56 |
| R70479 | TNFAIP3 | 0.55 |
| AA181306 | ST3GAL6 | 0.55 |
| T68169 | IRF2BP2 | 0.55 |
| AA056693 | PPAP2B | 0.55 |
| AI090289 | KLHL24 | 0.55 |
| H11063 | ZNF532 | 0.55 |
| N62553 | SLC22A9 | 0.54 |
| AA194143 | LOC51315 | 0.53 |
| H22927 | OSBPL1A | 0.53 |
| AA886758 | C1ORF24 | 0.53 |
| AA705942 | HOOK3 | 0.53 |
| AI038466 | JMY | 0.53 |
| T91078 | LOC401321 | 0.53 |
| N26311 | GDF15 | 0.52 |
| AA071470 | WWC3 | 0.52 |
| N47445 | EPDR1 | 0.52 |
| AA699790 | RPL31 | 0.52 |
| T57349 | KLHL24 | 0.52 |
| AA682293 | PAH | 0.51 |
| AA701860 | FST | 0.51 |
| AI288235 | FLJ35282 | 0.51 |
| R67376 | PSCD3 | 0.51 |
| H94667 | LOC389831 | 0.51 |
| AA406535 | NDUFS1 | 0.51 |
| AA156749 | C21ORF57 | 0.50 |
| AA629688 | CACNA2D1 | 0.50 |
| AA884403 | CTF1 | 0.49 |
| H59805 | IGF2BP1 | 0.49 |
| R56894 | MARK1 | 0.49 |
| AA430668 | FCGRT | 0.49 |
| AA443903 | KCNN4 | 0.49 |
| T41078 | BAZ2B | 0.49 |
| R93719 | GSPT1 | 0.49 |
| AA464600 | MYC | 0.49 |
| AA909676 | PVT1 | 0.49 |
| N91921 | TRBC1 | 0.49 |
| AA709086 | TEAD1 | 0.48 |
| R53963 | SV2B | 0.48 |
| AA447525 | DZIP1 | 0.48 |
| AA975183 | THEM4 | 0.48 |
| AA426408 | SEZ6L2 | 0.48 |
| N80713 | CDKL5 | 0.48 |
| AA127069 | RIS1 | 0.48 |
| H29198 | PVT1 | 0.48 |
| AA171739 | FLJ20054 | 0.48 |
| AA677327 | ST3GAL6 | 0.47 |
| N94060 | LRIG3 | 0.47 |
| AA886999 | ZNF197 | 0.47 |
| AA425437 | IGSF3 | 0.47 |
| AA450189 | ENO2 | 0.47 |
| AA491292 | SLC39A10 | 0.47 |
| R62612 | FN1 | 0.47 |
| AA707388 | INPP4B | 0.47 |
| W85883 | FLJ10847 | 0.47 |
| AA677224 | FLJ13910 | 0.47 |
| AA677224 | LOC285074 | 0.47 |
| N26658 | TGFBR3 | 0.47 |
| W70230 | COPZ2 | 0.47 |
| H92504 | DDIT4 | 0.46 |
| AA253464 | DKK1 | 0.46 |
| AA664155 | ASAH1 | 0.46 |
| H98822 | ALS2CR2 | 0.46 |
| AA026120 | BHLHB2 | 0.46 |
| AA262235 | DDX26 | 0.46 |
| R81831 | ZNF217 | 0.46 |
| H96654 | WBP5 | 0.46 |
| N75713 | CYBRD1 | 0.46 |
| H17038 | FLJ25076 | 0.46 |
| AI026771 | SPRED1 | 0.45 |
| AI301365 | LOC389833 | 0.45 |
| AA460143 | GNPDA1 | 0.45 |
| W74602 | TEAD4 | 0.45 |
| AA284296 | MGC70863 | 0.45 |
| R43456 | UGCGL2 | 0.45 |
| AA148542 | STK38L | 0.45 |
| AA437223 | LOC153222 | 0.44 |
| AA933641 | FLJ20674 | 0.44 |
| AA778310 | CENTD3 | 0.44 |
| AA927734 | KIAA1217 | 0.44 |
| AI273699 | NBPF3 | 0.44 |
| AA478470 | DDAH1 | 0.44 |
| AI032307 | NA | 0.44 |
| AA634028 | HLA-DPA1 | 0.44 |
| R41933 | H3/O | 0.44 |
| R41933 | HIST1H2BC | 0.44 |
| R41933 | HIST1H2BD | 0.44 |
| R41933 | HIST1H2BE | 0.44 |
| R41933 | HIST1H2BF | 0.44 |
| R41933 | HIST1H2BG | 0.44 |
| R41933 | HIST1H2BI | 0.44 |
| R41933 | HIST1H2BO | 0.44 |
| R41933 | HIST2H3C | 0.44 |
| R41933 | RP5-998N21.6 | 0.44 |
| W73883 | PON2 | 0.44 |
| H22559 | FHOD3 | 0.44 |
| H57273 | PRCP | 0.44 |
| AA279467 | RPL23AP7 | 0.44 |
| H57119 | LOC151877 | 0.44 |
| N45138 | TGFB2 | 0.44 |
| AA609170 | FLJ44653 | 0.43 |
| N77198 | NA | 0.43 |
| N57906 | FLJ36166 | 0.43 |
| N21550 | KIAA1922 | 0.43 |
| N72256 | ZADH2 | 0.43 |
| R63497 | LOC349114 | 0.43 |
| AA461427 | GAS6 | 0.43 |
| AA482230 | LDOC1L | 0.42 |
| AA125825 | ACVR2A | 0.42 |
| N49629 | UBD | 0.42 |
| AA977196 | TMEM38A | 0.42 |
| AA910213 | ALS2CL | 0.42 |
| H96982 | RFP2 | 0.42 |
| N45114 | ZNF322A | 0.42 |
| AI364148 | HMX1 | 0.42 |
| AI364148 | HMX2 | 0.42 |
| W74293 | MGC16037 | 0.42 |
| AA703159 | WDSUB1 | 0.42 |
| AA497051 | ST6GALNAC2 | 0.41 |
| AI140978 | HIPK2 | 0.41 |
| AI239814 | MYB | 0.41 |
| R85387 | AK3L1 | 0.41 |
| R85387 | AK3L2 | 0.41 |
| N72891 | SOS1 | 0.41 |
| AA953560 | FN1 | 0.41 |
| AA446994 | FGFR4 | 0.41 |
| AA668457 | TYRP1 | 0.41 |
| N68012 | CLK4 | 0.41 |
| R52703 | TK2 | 0.41 |
| N92749 | FAM102A | 0.41 |
| AA995282 | FHL2 | 0.41 |
| W07745 | ZADH2 | 0.41 |
| AI336456 | LOC402560 | 0.41 |
| AI221974 | NA | 0.41 |
| AI498125 | PVT1 | 0.41 |
| AI498125 | KLF6 | 0.41 |
| AA055585 | CRY1 | 0.40 |
| R19031 | APBB1 | 0.40 |
| H28119 | NA | 0.40 |
| AA873427 | SOS1 | 0.40 |
| T54672 | LOC492311 | 0.40 |
| R49013 | FLJ38028 | 0.40 |
| AI264427 | UNQ5783 | 0.40 |
| AA702676 | KIAA1443 | 0.40 |
| H79035 | PPP1R3E | 0.40 |
| H79035 | ZD52F10 | 0.40 |
| AA457707 | SSPO | 0.40 |
| AA167386 | KRT18 | 0.40 |
| AA664179 | PDCD6IP | 0.40 |
| R82991 | IRF2 | 0.40 |
| AA393214 | GUCY1A3 | 0.40 |
| AI131266 | ENAH | 0.40 |
| R53928 | HIP1 | 0.40 |
| AI150389 | CXORF44 | 0.39 |
| AA489655 | FLJ36166 | 0.39 |
| AI221371 | HLF | 0.39 |
| R59192 | ANKS6 | 0.39 |
| R72185 | LRP6 | 0.39 |
| AA126261 | INSR | 0.39 |
| T47312 | SUPV3L1 | 0.39 |
| AA046407 | TBC1D4 | 0.39 |
| AA400457 | ZNF135 | 0.39 |
| T91160 | PPP2R3A | 0.39 |
| T89372 | KIAA1161 | 0.39 |
| T52700 | LOC130940 | 0.39 |
| H11987 | GAL | 0.39 |
| AI623173 | TRBC1 | 0.39 |
| T64380 | MGAT3 | 0.39 |
| AA421473 | BRWD2 | 0.39 |
| AA432080 | ANKRD20A1 | 0.38 |
| AI301576 | PDCD6 | 0.38 |
| R53951 | PLD1 | 0.38 |
| R97756 | NA | 0.38 |
| AA436174 | PSCD3 | 0.38 |
| AA629264 | DDX12 | 0.38 |
| AA402879 | ZNF638 | 0.38 |
| AI290275 | MARS | 0.38 |
| AA015892 | SPAG9 | 0.38 |
| AA399253 | C10ORF47 | 0.38 |
| AI288965 | NR1D1 | 0.38 |
| AA453202 | RNF144 | 0.38 |
| W95118 | ITPKB | 0.38 |
| R94153 | C21ORF57 | 0.38 |
| H19234 | DEXI | 0.38 |
| T68845 | TRIM4 | 0.37 |
| AA663944 | SERPINH1 | 0.37 |
| R71440 | TRUB1 | 0.37 |
| AA418614 | ATP9A | 0.37 |
| AA436260 | ATF5 | 0.37 |
| AA872311 | FLJ14082 | 0.37 |
| H96630 | KCNC4 | 0.37 |
| AA151374 | UTS2D | 0.37 |
| N23399 | LHFPL3 | 0.37 |
| N29986 | HSPA14 | 0.37 |
| AA417742 | LARP1 | 0.37 |
| AA972120 | TM6SF1 | 0.37 |
| H97413 | TCF7 | 0.37 |
| AA480071 | TSPAN14 | 0.37 |
| AA128362 | KIAA0853 | 0.37 |
| AI247518 | C11ORF49 | 0.37 |
| AA634132 | GPIAP1 | 0.37 |
| N92134 | MRPS30 | 0.37 |
| AA917821 | PAPPA | 0.37 |
| AA708613 | CD44 | 0.37 |
| AI221846 | WBSCR16 | 0.36 |
| AA911034 | LMO7 | 0.36 |
| H22826 | C12ORF60 | 0.36 |
| AI301111 | PLCG1 | 0.36 |
| R76365 | TPP1 | 0.36 |
| AA664004 | NNT | 0.36 |
| AA625804 | TRAF3IP3 | 0.36 |
| AA676760 | APOLD1 | 0.36 |
| AA432292 | RPS6KA5 | 0.36 |
| N31641 | RPS24 | 0.36 |
| AA626146 | ZCCHC4 | 0.36 |
| R91215 | GRAMD1B | 0.36 |
| AA427719 | TWSG1 | 0.36 |
| AA486182 | C1ORF24 | 0.36 |
| AA191493 | C20ORF112 | 0.36 |
| AA912199 | DSG2 | 0.36 |
| W37448 | TXLNA | 0.36 |
| N34055 | MRC2 | 0.36 |
| H52232 | GPM6B | 0.36 |
| AA284329 | AXIN2 | 0.36 |
| AA976642 | ACTR1B | 0.36 |
| AA682260 | EP400NL | 0.36 |
| H15844 | SEPTIN7 | 0.36 |
| AA633993 | C14ORF149 | 0.35 |
| AA406573 | NID1 | 0.35 |
| AA709414 | NAT9 | 0.35 |
| AI266693 | SYNGR1 | 0.35 |
| W90588 | PHLPPL | 0.35 |
| AA417700 | CKLF | 0.35 |
| AA455042 | RCN1 | 0.35 |
| AA181643 | PANK1 | 0.35 |
| AI091817 | MAP1B | 0.35 |
| AA670382 | NA | 0.35 |
| AA865227 | FBXO32 | 0.35 |
| AA046700 | IGFBP5 | 0.35 |
| H08560 | CABC1 | 0.35 |
| H73777 | MGC12966 | 0.35 |
| AA476584 | SERPINE2 | 0.35 |
| N59721 | SLA/LP | 0.35 |
| AA489061 | ZNF41 | 0.35 |
| AA278721 | MAPK1 | 0.35 |
| W45690 | ZKSCAN1 | 0.35 |
| AA009763 | NUFIP2 | 0.35 |
| AA424756 | TMEM77 | 0.35 |
| H57959 | FMNL3 | 0.35 |
| H17909 | LOC133308 | 0.35 |
| H10673 | CRABP1 | 0.35 |
| AA421218 | SUMO1 | 0.35 |
| AA488626 | LOC196394 | 0.35 |
| R89365 | ZBTB8OS | 0.35 |
| AA778570 | PCGF3 | 0.35 |
| N95112 | FAM62B | 0.35 |
| R22340 | HCP1 | 0.35 |
| W45014 | BRI3BP | 0.34 |
| AA927474 | TWSG1 | 0.34 |
| N91767 | IL16 | 0.34 |
| AI300782 | SPRY4 | 0.34 |
| AA425382 | ZNF154 | 0.34 |
| AA504346 | EVL | 0.34 |
| R20625 | ATP5G2 | 0.34 |
| AA455126 | FAM92A1 | 0.34 |
| AA626363 | APPBP2 | 0.34 |
| AA046411 | KLF12 | 0.34 |
| H14569 | NPEPL1 | 0.34 |
| AA778640 | PTK2 | 0.34 |
| AI126054 | LOC153222 | 0.34 |
| N50563 | SPAG9 | 0.34 |
| N58144 | KIAA1706 | 0.34 |
| AA779937 | RSBN1L | 0.34 |
| AA886236 | RBM20 | 0.34 |
| AA668300 | PANK1 | 0.34 |
| H14604 | THYN1 | 0.34 |
| AA487902 | C10ORF58 | 0.34 |
| N71061 | JAG2 | 0.34 |
| AA906952 | CAMSAP1L1 | 0.34 |
| AA406094 | RGS3 | 0.34 |
| T85176 | KIAA0804 | 0.34 |
| AI077781 | PVT1 | 0.33 |
| W05002 | IL24 | 0.33 |
| AA281635 | RXRA | 0.33 |
| AA464615 | HLA-DQB1 | 0.33 |
| AA669055 | HLA-DQB2 | 0.33 |
| AA669055 | LOC284804 | 0.33 |
| AI150318 | INSR | 0.33 |
| AI248048 | HNRPLL | 0.33 |
| AI205918 | SMC6L1 | 0.33 |
| AA700010 | EML4 | 0.33 |
| AA122022 | KIF21A | 0.33 |
| AA872404 | SLC14A2 | 0.33 |
| AA961252 | C5 | 0.33 |
| AA780059 | CRELD1 | 0.33 |
| AI672251 | EIF4EBP1 | 0.33 |
| AI369144 | RRAGD | 0.33 |
| N54401 | TIMP2 | 0.33 |
| AA486280 | CLCN3 | 0.33 |
| N45115 | NA | 0.33 |
| AA912204 | ZNF42 | 0.33 |
| AI300989 | ITCH | 0.33 |
| AA864919 | DVL3 | 0.33 |
| W84790 | TMEM49 | 0.33 |
| AA159669 | ERO1L | 0.33 |
| AA457116 | JARID1B | 0.33 |
| AA481769 | RBMS3 | 0.33 |
| AI128422 | ADAM15 | 0.32 |
| AA292676 | NOVA1 | 0.32 |
| AI362062 | MGC13170 | 0.32 |
| AA430409 | EIF2C2; AGO2 | 0.32 |
| AI263575 | FOXO3A | 0.32 |
| AA176819 | C5ORF4 | 0.32 |
| AA406354 | KIAA1922 | 0.32 |
| AA443695 | SBNO1 | 0.32 |
| AA984682 | THRAP2 | 0.32 |
| AA449326 | ZNF532 | 0.32 |
| H80749 | TTC17 | 0.32 |
| AA194019 | VPS24 | 0.32 |
| AI206412 | AKAP9 | 0.32 |
| AA774104 | ABLIM1 | 0.32 |
| AA406601 | TRIM31 | 0.32 |
| AA054421 | HIST2H2AA | 0.32 |
| AA436252 | FNBP4 | 0.32 |
| AA872279 | FLJ34306 | 0.32 |
| H85475 | PSG3 | 0.32 |
| H12630 | PSG8 | 0.32 |
| H12630 | C14ORF43 | 0.32 |
| R95913 | BCL7A | 0.32 |
| H90147 | FLNC | 0.32 |
| AI675658 | IL10RB | 0.32 |
| R67983 | KIAA1683 | 0.32 |
| H96597 | NF1 | 0.32 |
| AA489040 | IGFBPL1 | 0.32 |
| AA620528 | C14ORF119 | 0.32 |
| W88562 | LIPG | 0.31 |
| AA599574 | PDCD6IP | 0.31 |
| AA055218 | CAV1 | 0.31 |
| AA055835 | C20ORF121 | 0.31 |
| AA669593 | PPP1R10 | 0.31 |
| AA071526 | TMEM77 | 0.31 |
| N34764 | LTC4S | 0.31 |
| AI299075 | MAK3 | 0.31 |
| H16725 | SNX16 | 0.31 |
| AA969394 | TMEM135 | 0.31 |
| N69100 | FLI1 | 0.31 |
| AI288838 | TRIM4 | 0.31 |
| N27415 | SDCBP | 0.31 |
| AA456109 | TBC1D3 | 0.31 |
| AA708275 | TBC1D3B | 0.31 |
| AA708275 | RAB6IP1 | 0.31 |
| R60711 | IRF2BP2 | 0.31 |
| N73222 | CXX1 | 0.31 |
| W72596 | ZNF337 | 0.31 |
| AA705436 | NA | 0.31 |
| AI097452 | TMEM42 | 0.31 |
| AA479205 | GM2A | 0.31 |
| AA453978 | FUCA1 | 0.31 |
| N95761 | RAB10 | 0.31 |
| AA709001 | TRIP6 | 0.31 |
| AA485677 | ASXL1 | 0.31 |
| N64780 | OBSL1 | 0.31 |
| AA430576 | LAP3 | 0.31 |
| AA757812 | CPNE1 | 0.31 |
| AA481034 | RBM12 | 0.31 |
| AA481034 | NA | 0.31 |
| AI151359 | LANCL2 | 0.31 |
| AA864439 | NFE2L1 | 0.31 |
| AA496576 | COX11 | 0.30 |
| AA457644 | LRRN3 | 0.30 |
| N36948 | LGALS3 | 0.30 |
| AA630328 | RXRA | 0.30 |
| AA777229 | ZNF585A | 0.30 |
| AA970119 | TMEM18 | 0.30 |
| AA857941 | LOC389765 | 0.30 |
| AA975005 | LRP6 | 0.30 |
| N99539 | ITIH3 | 0.30 |
| T68035 | CLDN10 | 0.30 |
| R54559 | EPB41 | 0.30 |
| AA987359 | GAS6 | 0.30 |
| R76863 | LGALS3 | 0.30 |
| AI221769 | ABL1 | 0.30 |
| AA496785 | GNPTAB | 0.30 |
| AA788772 | BRRN1 | −0.30 |
| N54344 | BLVRA | −0.30 |
| AA192419 | KIAA1212 | −0.30 |
| AA497044 | U2AF1 | −0.30 |
| AA448694 | TMEM108 | −0.30 |
| AA973654 | TNKS | −0.30 |
| AI241421 | MYL4 | −0.30 |
| AA705225 | DNAJC3 | −0.30 |
| AA927453 | SPATA3 | −0.30 |
| AI125254 | LSM3 | −0.30 |
| AA461098 | SERPINB8 | −0.30 |
| W61361 | C9ORF52 | −0.30 |
| N69066 | COG5 | −0.30 |
| AA912461 | CHD9 | −0.30 |
| W46341 | ZNF117 | −0.30 |
| H65481 | CCNL1 | −0.30 |
| AA465166 | DLG7 | −0.30 |
| AA262211 | PHKB | −0.30 |
| AI285180 | C14ORF32 | −0.30 |
| H58992 | ARG1 | −0.30 |
| R93602 | SPHK1 | −0.30 |
| AI341901 | GNG2 | −0.30 |
| N26108 | ERO1LB | −0.31 |
| AI241301 | RUFY2 | −0.31 |
| R39039 | TATDN3 | −0.31 |
| AA906896 | PPM2C | −0.31 |
| H11036 | FRS2 | −0.31 |
| T71650 | SELPLG | −0.31 |
| AA954738 | NUDCD3 | −0.31 |
| R43544 | MAGED2 | −0.31 |
| AI684984 | TRIM33 | −0.31 |
| AA426120 | INADL | −0.31 |
| AA005153 | ITGA4 | −0.31 |
| H79341 | EXT1 | −0.31 |
| H63223 | HOOK1 | −0.31 |
| AA644183 | TAF1B | −0.31 |
| AA620887 | CROP | −0.31 |
| AA447587 | PLSCR1 | −0.31 |
| AI049711 | TAF1B | −0.31 |
| R32478 | Unknown | −0.31 |
| AA907052 | SFMBT2 | −0.31 |
| AA890093 | CYBB | −0.31 |
| AA463492 | NR4A1 | −0.31 |
| N94487 | PLK4 | −0.31 |
| AA732873 | C10ORF70 | −0.31 |
| AA431199 | C2ORF32 | −0.31 |
| R07066 | KIF13B | −0.31 |
| W86466 | MAK3 | −0.31 |
| AA678176 | ITGA9 | −0.31 |
| AA865557 | KIF11 | −0.31 |
| AA504625 | TOR1AIP1 | −0.31 |
| AI342950 | KIAA0146 | −0.31 |
| AA904593 | REEP5 | −0.31 |
| AA677078 | ASPHD2 | −0.31 |
| H17273 | AKAP14 | −0.31 |
| AA400121 | FLJ11000 | −0.31 |
| R16019 | OMG | −0.31 |
| N47511 | MRPL21 | −0.31 |
| AA454566 | DCP1A | −0.31 |
| AI305162 | AGPAT4 | −0.31 |
| AA700783 | NA | −0.31 |
| AI247377 | ADORA2A | −0.31 |
| N57553 | C1ORF82 | −0.31 |
| AI147399 | TXNDC13 | −0.31 |
| AA007516 | PSMC3 | −0.31 |
| AA282230 | ELL3 | −0.31 |
| AA464143 | LRRC40 | −0.31 |
| AA456020 | KIAA1212 | −0.31 |
| AI022231 | GLIPR1 | −0.31 |
| AI129398 | PPP3CA | −0.31 |
| AA121266 | C17ORF27 | −0.31 |
| H17861 | QKI | −0.31 |
| N66624 | CCNC | −0.31 |
| AA453231 | UQCR | −0.31 |
| AA629862 | MTF1 | −0.31 |
| AA448256 | ADD3 | −0.31 |
| AA461325 | C6ORF68 | −0.31 |
| H26324 | LSAMP | −0.32 |
| R49462 | COL4A4 | −0.32 |
| AA630485 | FUS | −0.32 |
| AA779569 | EPB41L2 | −0.32 |
| W88572 | ASF1A | −0.32 |
| AI198924 | MID1 | −0.32 |
| AA460270 | IDH2 | −0.32 |
| AA679907 | PTGDS | −0.32 |
| R59579 | GABBR2 | −0.32 |
| AA775405 | SLC36A1 | −0.32 |
| AI222995 | SH3D19 | −0.32 |
| H86071 | KLRC3 | −0.32 |
| AA191156 | SCFD1 | −0.32 |
| AI218719 | C10ORF42 | −0.32 |
| AI086287 | DUSP5 | −0.32 |
| W65461 | KBTBD2 | −0.32 |
| W02624 | KIAA0913 | −0.32 |
| AA443585 | SLC44A1 | −0.32 |
| AA703582 | TMBIM4 | −0.32 |
| AA634291 | SECISBP2 | −0.32 |
| AA704707 | KCNJ8 | −0.32 |
| AA036956 | LRP11 | −0.32 |
| AA988586 | MGC52057 | −0.32 |
| AI239661 | RERE | −0.32 |
| H71242 | PPP1R2 | −0.32 |
| N52605 | SND1 | −0.32 |
| AA019547 | CCM2 | −0.32 |
| AA903402 | CIR | −0.32 |
| N73571 | ENY2 | −0.32 |
| AA011390 | WBP11 | −0.32 |
| AA130669 | ZNF273 | −0.32 |
| W86455 | BHLHB3 | −0.32 |
| AA485896 | GLRX | −0.32 |
| AA291163 | ARMC8 | −0.32 |
| R31524 | MSRA | −0.32 |
| AA994467 | SAMD9L | −0.32 |
| AA996042 | PPM1E | −0.32 |
| AA421267 | ZDHHC17 | −0.32 |
| W67243 | UMOD | −0.32 |
| AA886414 | NA | −0.32 |
| AA934401 | MEF2A | −0.32 |
| AA491228 | ZNF318 | −0.32 |
| AI004484 | PPM2C | −0.32 |
| AI080633 | FLJ43663 | −0.32 |
| AI248213 | PDE4DIP | −0.32 |
| N73278 | DOCK8 | −0.33 |
| AA400074 | PBX1 | −0.33 |
| AI126071 | MBNL1 | −0.33 |
| AA131516 | GPR146 | −0.33 |
| H23521 | GLT28D1 | −0.33 |
| AA045665 | LOC388630 | −0.33 |
| AA625812 | TSHZ2 | −0.33 |
| AA676649 | FKBP14 | −0.33 |
| AA733022 | PSD4 | −0.33 |
| W90716 | HDAC9 | −0.33 |
| AA629911 | MGC24039 | −0.33 |
| AA703480 | LOC128977 | −0.33 |
| AA927761 | CCDC26 | −0.33 |
| AA628201 | SPATA5L1 | −0.33 |
| AA451905 | XPR1 | −0.33 |
| AA453474 | TRPM4 | −0.33 |
| AA932133 | TMEM16F | −0.33 |
| AI016000 | SOCS3 | −0.33 |
| AA001219 | CDGAP | −0.33 |
| AA425435 | PPP2R5C | −0.33 |
| AI336804 | HS3ST1 | −0.33 |
| T55714 | ANKRD11 | −0.33 |
| AI219775 | DNAH11 | −0.33 |
| AA490887 | MFAP3L | −0.33 |
| AA398341 | TCF7L2 | −0.33 |
| AI268824 | RABGEF1 | −0.33 |
| AA135638 | HK1 | −0.33 |
| AA703577 | KCNJ15 | −0.33 |
| AI094257 | SSBP3 | −0.33 |
| AA775212 | PGGT1B | −0.33 |
| AA989220 | TPMT | −0.33 |
| AA677257 | C1ORF48 | −0.33 |
| R38208 | SOX4 | −0.33 |
| AA029415 | ARL5B | −0.33 |
| AA922226 | AKAP7 | −0.33 |
| R89082 | GRAMD1C | −0.33 |
| AA625897 | C11ORF51 | −0.33 |
| AA476235 | XPNPEP1 | −0.33 |
| AA453477 | HMGN2 | −0.34 |
| AI219528 | HTR4 | −0.34 |
| T86959 | SH3TC2 | −0.34 |
| T86959 | NASP | −0.34 |
| AA702432 | DECR1 | −0.34 |
| H72937 | ASPH | −0.34 |
| W02677 | C14ORF100 | −0.34 |
| H17648 | RASGRP1 | −0.34 |
| AA278633 | UBE2E2 | −0.34 |
| AA626236 | OLIG2 | −0.34 |
| AI360012 | KIAA1961 | −0.34 |
| AI018807 | PSMD12 | −0.34 |
| AA497132 | EHBP1 | −0.34 |
| H60119 | SGOL2 | −0.34 |
| AA682533 | UTRN | −0.34 |
| R93745 | DYRK1A | −0.34 |
| AA480865 | ARHGAP18 | −0.34 |
| AI040624 | API5 | −0.34 |
| AA778847 | LRRC1 | −0.34 |
| R79962 | TSSK4 | −0.34 |
| AI075923 | KIAA1524 | −0.34 |
| AI248987 | MCM4 | −0.34 |
| R07012 | P2RX5 | −0.34 |
| AA044267 | MRPS18C | −0.35 |
| N64429 | BPGM | −0.35 |
| AA678065 | HDHD1A | −0.35 |
| R38639 | CHD7 | −0.35 |
| AA644224 | C14ORF108 | −0.35 |
| AA906454 | SAMHD1 | −0.35 |
| AA421603 | ZNF652 | −0.35 |
| AA706892 | NA | −0.35 |
| W58325 | CCL7 | −0.35 |
| AA040170 | JAG1 | −0.35 |
| R70685 | FLJ11021 | −0.35 |
| AA291183 | HS3ST4 | −0.35 |
| AA878786 | PPIA | −0.35 |
| AI160166 | MYL6 | −0.35 |
| AA488346 | SOX4 | −0.35 |
| AA453420 | TP53BP2 | −0.35 |
| N34418 | RAB18 | −0.35 |
| AA156821 | TAP2 | −0.35 |
| AA406373 | USP15 | −0.35 |
| N79180 | USP50 | −0.35 |
| AA399952 | PDLIM5 | −0.35 |
| AA432103 | LRP2 | −0.35 |
| AI282079 | RORA | −0.35 |
| AA432137 | MTMR2 | −0.35 |
| AA933721 | RNF6 | −0.35 |
| AI242096 | MBIP | −0.35 |
| AI273507 | MORF4L2 | −0.35 |
| AA947294 | SND1 | −0.35 |
| AI243340 | SNX2 | −0.35 |
| AI191446 | SLC35F3 | −0.35 |
| AI032301 | DDX58 | −0.35 |
| AA126958 | FLJ31033 | −0.35 |
| AA922376 | CLCF1 | −0.35 |
| AI040033 | PSMA6 | −0.35 |
| AA047338 | WDR43 | −0.35 |
| AA460557 | SPTLC2 | −0.35 |
| AA160852 | H2-ALPHA | −0.35 |
| AA626698 | CD160 | −0.35 |
| AA463248 | KIAA0226 | −0.35 |
| W94774 | WFDC6 | −0.36 |
| AA626362 | CDC2L6 (CDV- | −0.36 |
| | 1) | |
| H92525 | RTP4 | −0.36 |
| N23400 | FLJ35725 | −0.36 |
| AA157001 | ROM1 | −0.36 |
| H84113 | JAK1 | −0.36 |
| AA284634 | PLN | −0.36 |
| AA427940 | PAPD4 | −0.36 |
| T81837 | MICAL2 | −0.36 |
| AA778856 | FNDC3B | −0.36 |
| H89725 | SFRS10 | −0.36 |
| AI583623 | GTDC1 | −0.36 |
| AI078828 | VAPA | −0.36 |
| H16686 | PCTK2 | −0.36 |
| AI217248 | DEADC1 | −0.36 |
| AA702788 | MAK3 | −0.36 |
| AA777399 | NR4A3 | −0.36 |
| N72196 | BSPRY | −0.36 |
| W38022 | CNKSR2 | −0.36 |
| R40781 | SERPINB1 | −0.36 |
| AA486275 | C9ORF95 | −0.36 |
| AA464603 | MGC15912 | −0.36 |
| AI127483 | SERPINI1 | −0.36 |
| AA115876 | ATRX | −0.36 |
| AI292068 | C5ORF5 | −0.36 |
| AI348442 | PSMB9 | −0.36 |
| AA862434 | HSPA8 | −0.36 |
| AA629567 | Unknown | −0.36 |
| H97875 | YWHAG | −0.36 |
| R08938 | LOC92312 | −0.36 |
| AA970152 | ADPRH | −0.36 |
| AA418675 | PHC3 | −0.36 |
| AI168122 | MAGEF1 | −0.36 |
| AA425302 | TMEM87B | −0.36 |
| AA677461 | C1ORF186 | −0.36 |
| T91042 | RAB2 | −0.36 |
| AA677106 | ANKRD28 | −0.36 |
| N25798 | MRPS14 | −0.36 |
| AI221939 | TTC17 | −0.36 |
| AI028308 | GLRA3 | −0.36 |
| AA455624 | NOSTRIN | −0.36 |
| N74106 | ZNF441 | −0.37 |
| AI088742 | GRHL3 | −0.37 |
| AI017149 | C13ORF12 | −0.37 |
| R38655 | APC | −0.37 |
| AI185458 | C12ORF30 | −0.37 |
| AI733697 | TMEM23 | −0.37 |
| T55587 | STK16 | −0.37 |
| R49144 | LOC441052 | −0.37 |
| R38894 | ALOX5 | −0.37 |
| H51574 | RIMS4 | −0.37 |
| AI242542 | PELI1 | −0.37 |
| W86504 | PARP11 | −0.37 |
| AA608880 | NCL | −0.37 |
| N90109 | SYNE2 | −0.37 |
| AA922060 | MT1F | −0.37 |
| T56281 | ACACA | −0.37 |
| N74920 | LGP2 | −0.37 |
| AA455279 | KIAA2018 | −0.37 |
| AA446456 | ARID5B | −0.37 |
| AA135616 | MMAA | −0.37 |
| H15522 | NA | −0.37 |
| AI123790 | CUL3 | −0.37 |
| AA995108 | ALMS1 | −0.37 |
| AA694488 | PABPC1 | −0.37 |
| AI222165 | PCGF5 | −0.37 |
| AA136060 | ELK3 | −0.37 |
| AA040699 | ENDOD1 | −0.38 |
| AA918646 | ADORA2A | −0.38 |
| AI289840 | HNRPD | −0.38 |
| T55592 | FBXO43 | −0.38 |
| AA620638 | RGL1 | −0.38 |
| AA683557 | GABRB1 | −0.38 |
| R24969 | FTS | −0.38 |
| AI217765 | SH3D19 | −0.38 |
| AA976599 | KLF12 | −0.38 |
| W84891 | LEREPO4 | −0.38 |
| AA777255 | USP15 | −0.38 |
| R92011 | UBE2J1 | −0.38 |
| N57554 | RASSF6 | −0.38 |
| AA921679 | BLZF1 | −0.38 |
| R43576 | KLHL14 | −0.38 |
| AI051108 | ACSL3 | −0.38 |
| AA788780 | ABHD5 | −0.38 |
| AI241278 | IPO11 | −0.38 |
| AA195041 | EPC1 | −0.38 |
| N49717 | C2ORF34 | −0.38 |
| AA922097 | ANGPTL1 | −0.38 |
| N31935 | POSTN | −0.38 |
| AI262129 | AMD1 | −0.38 |
| R82299 | INOC1 | −0.38 |
| AI015577 | BID | −0.38 |
| AA936138 | PTPRC | −0.38 |
| AA904360 | PIK3R3 | −0.38 |
| AI394701 | PTPN1 | −0.38 |
| R06605 | BACH1 | −0.39 |
| AI336948 | ELMO1 | −0.39 |
| AI090439 | UBE2A | −0.39 |
| AI248210 | USP53 | −0.39 |
| W37628 | MYADM | −0.39 |
| AA699589 | ELL2 | −0.39 |
| T87150 | ZNF6 | −0.39 |
| AA928817 | CHIC1 | −0.39 |
| AI275092 | CTSC | −0.39 |
| AA644088 | UGCGL1 | −0.39 |
| R89313 | CASK | −0.39 |
| AA045965 | UTP15 | −0.39 |
| AI222077 | LRAP | −0.39 |
| AA897402 | IL10RA | −0.39 |
| AA437226 | FBXL17 | −0.39 |
| H75459 | SH3RF1 | −0.39 |
| AA485676 | VEZT | −0.39 |
| AA425770 | ABC1 | −0.39 |
| AI022472 | ZNF514 | −0.39 |
| AA504273 | ACTL7B | −0.39 |
| AA634289 | FLJ11000 | −0.39 |
| H50656 | MAN1A1 | −0.39 |
| AA489636 | FAM49B | −0.39 |
| AA173423 | NETO2 | −0.39 |
| AA456821 | DPP4 | −0.40 |
| W70234 | ITFG1 | −0.40 |
| AA778241 | AFF1 | −0.40 |
| AA004412 | SLIT2 | −0.40 |
| AA489463 | RAB4A | −0.40 |
| H59921 | LRRC41 | −0.40 |
| AI217767 | KIAA1524 | −0.40 |
| AA167270 | BIRC6 | −0.40 |
| AI215937 | PCBP2 | −0.40 |
| AA504356 | NA | −0.40 |
| AA454591 | CCDC50 | −0.40 |
| N95059 | BIRC4BP | −0.40 |
| AA142842 | SPRED1 | −0.40 |
| AA677280 | TLR4 | −0.40 |
| AI371874 | MARCH6 | −0.40 |
| H78349 | BCLAF1 | −0.40 |
| H21107 | KIAA0226 | −0.40 |
| N36389 | MAOA | −0.40 |
| AA011096 | C6ORF173 | −0.41 |
| W90323 | ZFP30 | −0.41 |
| AA668204 | POPDC3 | −0.41 |
| H84369 | ACTA2 | −0.41 |
| T60048 | ACTG2 | −0.41 |
| T60048 | LCP1 | −0.41 |
| W73144 | BTG1 | −0.41 |
| N51323 | PFKFB2 | −0.41 |
| R16146 | TMEM50B | −0.41 |
| W69669 | CCDC23 | −0.41 |
| R89849 | TSC22D2 | −0.41 |
| N45223 | CYP2J2 | −0.41 |
| H09076 | DUSP18 | −0.41 |
| AI299221 | KBTBD8 | −0.41 |
| AA278766 | MARCH7 | −0.41 |
| N72288 | SAT | −0.41 |
| AA598631 | GPR137B | −0.42 |
| R39926 | SGTB | −0.42 |
| AA452545 | C1GALT1 | −0.42 |
| N73031 | CCDC50 | −0.42 |
| AA701978 | ELAC1 | −0.42 |
| N52912 | CYP4V2 | −0.42 |
| W90457 | GNAI1 | −0.42 |
| AA406420 | HNRPD | −0.42 |
| AA609738 | C13ORF7 | −0.42 |
| AA491265 | DST | −0.42 |
| N67598 | LANCL2 | −0.42 |
| T64972 | NAG8 | −0.42 |
| AA883504 | USP6NL | −0.42 |
| AA281137 | SGOL2 | −0.42 |
| AI262665 | CECR1 | −0.43 |
| AI342751 | ARL5B | −0.43 |
| AA281729 | REV3L | −0.43 |
| AA708786 | OSTF1 | −0.43 |
| AA149226 | AXUD1 | −0.43 |
| AA872011 | RHOA | −0.43 |
| AI028234 | TUBB2A | −0.43 |
| AI672565 | FAF1 | −0.43 |
| AA977210 | ZCCHC6 | −0.43 |
| AA705324 | SPTY2D1 | −0.43 |
| AA906879 | PFTK1 | −0.43 |
| AA704460 | RB1 | −0.43 |
| AA045192 | COX4NB | −0.44 |
| AI301207 | TCF2 | −0.44 |
| AI244667 | GPR65 | −0.44 |
| T86932 | TMEM30A | −0.44 |
| AI150297 | C1ORF21 | −0.44 |
| AI335359 | RGL1 | −0.44 |
| T98762 | SERPINB8 | −0.44 |
| AA972628 | SP3 | −0.44 |
| AA912705 | HBEGF | −0.44 |
| R14663 | GPR177 | −0.44 |
| AA001918 | MIER1 | −0.44 |
| AA001918 | HNRPD | −0.44 |
| H82104 | RAB30 | −0.44 |
| AI290596 | SSH2 | −0.44 |
| AA975530 | RGL1 | −0.44 |
| AI038592 | PDLIM5 | −0.44 |
| AA443846 | WDR72 | −0.44 |
| AA205598 | NR4A3 | −0.44 |
| H37761 | HSPA8 | −0.44 |
| AA620511 | DNMT2 | −0.44 |
| R95732 | IDS | −0.45 |
| H13205 | HLF | −0.45 |
| AI248021 | CREM | −0.45 |
| AA626724 | PTPRG | −0.45 |
| R38343 | SIPA1L2 | −0.45 |
| AA464598 | DPYD | −0.45 |
| W49559 | GBP4 | −0.45 |
| AI268082 | RNF139 | −0.45 |
| AA455970 | HRSP12 | −0.45 |
| W02265 | PPP1CB | −0.45 |
| AA876421 | MGAT5B | −0.45 |
| R88297 | GLS | −0.45 |
| W72090 | USP6NL | −0.45 |
| AA281137 | LMBRD1 | −0.45 |
| N62401 | EFHA2 | −0.45 |
| AI016151 | IL1RN | −0.45 |
| T72877 | JMJD2C | −0.46 |
| H56961 | TOR1AIP1 | −0.46 |
| W15521 | BIN3 | −0.46 |
| H96791 | NFIL3 | −0.46 |
| AA633811 | ETV6 | −0.46 |
| AI336785 | ERO1LB | −0.46 |
| H19429 | DST | −0.46 |
| H44784 | FLJ11000 | −0.46 |
| AI266442 | RP5-821D11.2 | −0.46 |
| AI264565 | KIAA1240 | −0.46 |
| H75690 | CAMK2D | −0.46 |
| W30935 | FLJ11021 | −0.46 |
| AI209205 | MAP2K6 | −0.46 |
| H07920 | CRIM1 | −0.46 |
| AA778314 | CCDC50 | −0.46 |
| H61552 | SHRM | −0.47 |
| R31831 | RNF111 | −0.47 |
| AA865355 | MOBK1B | −0.47 |
| AA210701 | SYT11 | −0.47 |
| R87238 | TSC22D1 | −0.47 |
| AA664389 | ADD2 | −0.47 |
| AA448280 | PTPN22 | −0.47 |
| AA906845 | TMEM59 | −0.47 |
| T64931 | TXNDC5 | −0.47 |
| T85185 | IFIT3 | −0.47 |
| N51761 | PTPRC | −0.47 |
| H74265 | TUBA3 | −0.47 |
| AA865469 | CECR1 | −0.47 |
| AA293496 | CYP4V2 | −0.47 |
| AA455986 | CLEC2D | −0.47 |
| H66883 | MAP3K5 | −0.47 |
| AI268273 | PRSS23 | −0.47 |
| AA431796 | ELK3 | −0.48 |
| N48701 | CCNA2 | −0.48 |
| AA459213 | CLEC2D | −0.48 |
| AI302421 | MBNL2 | −0.48 |
| AA285053 | STX3A | −0.48 |
| AI359037 | EPC1 | −0.48 |
| H54779 | SERPINB1 | −0.48 |
| R54664 | CLEC2D | −0.48 |
| N67007 | ARHGAP30 | −0.48 |
| W72330 | STK4 | −0.48 |
| AA455248 | TCF2 | −0.49 |
| AA699573 | KLRC4 | −0.49 |
| AA903175 | KLRK1 | −0.49 |
| AA903175 | ADCK2 | −0.49 |
| H06508 | SSBP3 | −0.49 |
| W91960 | COX7B2 | −0.49 |
| AI138368 | MCTP2 | −0.49 |
| AA206614 | ACTR3 | −0.49 |
| AA456112 | PRKACB | −0.49 |
| AA459980 | CASP7 | −0.49 |
| T50828 | PARP9 | −0.49 |
| N50904 | SORBS2 | −0.49 |
| AA987658 | SYNE2 | −0.49 |
| AI223295 | G1P3 | −0.49 |
| AA432030 | PTPN1 | −0.49 |
| W92859 | MASP2 | −0.50 |
| R56829 | TRIB1 | −0.50 |
| AI244972 | HSPC049 | −0.50 |
| N62857 | GABPB2 | −0.50 |
| AI093876 | GABPB2 | −0.50 |
| N48820 | EBF | −0.50 |
| AA917497 | PTEN | −0.50 |
| N67051 | EHD4 | −0.50 |
| AI149630 | LDHA | −0.50 |
| AA489611 | LARP5 | −0.50 |
| AA704941 | PHC3 | −0.51 |
| AA286777 | OSBPL3 | −0.51 |
| H10059 | GNG2 | −0.51 |
| AA620960 | TMEM23 | −0.51 |
| AA459293 | LOC440459 | −0.51 |
| AI016779 | IGFBP5 | −0.51 |
| T52830 | CAPN3 | −0.52 |
| AA278326 | FCGR2B | −0.52 |
| R68106 | CCNA2 | −0.52 |
| AA608568 | JAK1 | −0.52 |
| AI492016 | PAX5 | −0.52 |
| R16555 | HCST | −0.52 |
| AA699808 | RAPGEF2 | −0.53 |
| AA488969 | FABP1 | −0.53 |
| T53220 | NRP1 | −0.53 |
| AI285044 | ITM2B | −0.53 |
| AA453275 | C21ORF25 | −0.53 |
| AI674133 | ANGPTL1 | −0.53 |
| AA416740 | RAB30 | −0.53 |
| H99054 | SEMA6D | −0.53 |
| AA452824 | PRKACB | −0.53 |
| AA018980 | JAM3 | −0.54 |
| AA931102 | PFTK1 | −0.54 |
| T97353 | PRKAR2B | −0.54 |
| AA181500 | TMEM23 | −0.54 |
| H48346 | NET1 | −0.54 |
| R24543 | MEMO1 locus | −0.55 |
| AI076295 | MFSD2 | −0.55 |
| AA774524 | TUBA1 | −0.55 |
| AA180912 | CYBB | −0.55 |
| H72119 | ZNF138 | −0.55 |
| AA005196 | BHLHB9 | −0.56 |
| R20547 | ZNF407 | −0.56 |
| AA017242 | IFIT1 | −0.56 |
| AA489743 | PPAN | −0.56 |
| AI000807 | MAN2A1 | −0.56 |
| AA029052 | MEF2C | −0.56 |
| N49958 | NFKB1 | −0.56 |
| AI001741 | EPC1 | −0.57 |
| AA120875 | CD83 | −0.57 |
| AA111969 | ALOX5 | −0.57 |
| AI243516 | MYO6 | −0.57 |
| AA625890 | ITGAM | −0.57 |
| AA436187 | GLS | −0.58 |
| AA904684 | OAS2 | −0.59 |
| AA902449 | NA | −0.59 |
| H10156 | ELL2 | −0.60 |
| AA707219 | LBA1 | −0.60 |
| AA127794 | LOC143381 | −0.60 |
| AI024284 | LRP2BP | −0.61 |
| AI092008 | CCDC50 | −0.61 |
| AA902164 | TOX | −0.61 |
| AI250784 | PER3 | −0.62 |
| AA521459 | LOC391819 | −0.62 |
| AI018099 | FCGR2B | −0.62 |
| AA465663 | PRKCG | −0.62 |
| R89715 | TRIB1 | −0.62 |
| AI077990 | TLR4 | −0.63 |
| AI082399 | DNASE2B | −0.63 |
| AI820599 | FNDC3B | −0.64 |
| R45116 | SP100 | −0.64 |
| N21492 | EDN1 | −0.64 |
| H11003 | DACT1 | −0.65 |
| AA487274 | CD69 | −0.65 |
| AA279883 | EIF5 | −0.65 |
| H40023 | ARPC5L | −0.66 |
| AA909939 | CAMK2D | −0.66 |
| AA029441 | ARRDC3 | −0.66 |
| AA015658 | ITGAM | −0.66 |
| AA609962 | TLR7 | −0.66 |
| N30597 | KLF6 | −0.66 |
| AA416628 | G1P2 | −0.67 |
| AA406020 | RAPGEF2 | −0.67 |
| AA022908 | SLC16A1 | −0.67 |
| AA610081 | VEGFC | −0.67 |
| H07991 | SLC2A5 | −0.67 |
| H38650 | ERO1LB | −0.68 |
| H30558 | FAM46C | −0.68 |
| AA058597 | SGPP2 | −0.68 |
| AA962280 | KLHL24 | −0.68 |
| AA111979 | CD40 | −0.68 |
| AA886208 | SFRS10 | −0.68 |
| AA883496 | KIAA1509 | −0.69 |
| AA905404 | STX11 | −0.69 |
| R33851 | TOX | −0.70 |
| AA404337 | GBP2 | −0.70 |
| W77927 | PDE4B | −0.71 |
| AA453293 | ARRDC3 | −0.72 |
| AI091540 | KLF6 | −0.72 |
| AA865224 | PSCDBP | −0.73 |
| AA490903 | SYK | −0.73 |
| AA598572 | SERPINB9 | −0.73 |
| AA430512 | NCOA5 | −0.74 |
| AA521358 | TOX | −0.74 |
| AA972366 | SPIB | −0.74 |
| N71628 | COL3A1 | −0.76 |
| T98612 | CNR1 | −0.77 |
| R20626 | CLEC2B | −0.77 |
| AA417921 | TNFSF10 | −0.78 |
| H54629 | CD79B | −0.79 |
| R72079 | KLF6 | −0.80 |
| AA156946 | KIAA1432 | −0.80 |
| N47010 | SART2 | −0.80 |
| AA045278 | IL15 | −0.84 |
| N59270 | ZPBP | −0.84 |
| AA400474 | LOC442096 | −0.87 |
| N69453 | CD38 | −0.89 |
| R00276 | ITGA2 | −0.90 |
| AA463610 | ARRDC3 | −0.91 |
| R33609 | SYTL3 | −0.98 |
| AI091450 | SH3D19 | −0.98 |
| AA446651 | LOC91316 | −0.98 |
| H18423 | RALGPS2 | −0.99 |
| AA972030 | RASSF6 | −1.05 |
| N52073 | IGLV6-57 | −1.05 |
| AA971714 | IGLC1 | −1.09 |
| T67053 | IGLC2 | −1.10 |
| T67053 | IGLV2-14 | −1.10 |
| T67053 | PIP3-E | −1.10 |
| N48178 | IGLL1 | −1.13 |
| W73790 | STAT5B | −1.25 |
| AA282023 | C20ORF103 | −1.31 |
| R44985 | | −1.45 |

### Example 5

### Predictive Gene Classifier for Autism Spectrum Disorders

### Introduction:

This Example further demonstrates that several phenotypic variants of idiopathic autism can be distinguished from nonautistic controls on the basis of differential gene expression of limited sets of genes in lymphoblastoid cell lines (LCL) from the respective individuals with a predicted classification accuracy of up to 89.9% and identified a series of 20 transcripts that were differentially expressed among tested groups. The data suggests that such sets of genes may be useful biomarkers for diagnosis of idiopathic autism.

### Materials and Methods:

The materials and methods and analysis of data were performed as above for Example 4 Supra, with the only difference in the analyses was the exclusion of sibling controls from the analyses, since similar genotypes tend to blur the differences in gene expression profiles of related individuals.

### Results and Discussion:

A reanalysis of DNA microarray data of nonautistic controls vs. data from the combined autistic samples was done after removing all controls who were siblings of the autistic probands. As a result, 20 (instead of 5) novel transcripts were identified as differentially expressed (relative to controls) among all 3 subgroups (Table 28). Interestingly, all of these transcripts are found in intronic or intergenic regions of the chromosomes (suggestive of noncoding RNA), and the majority is also androgen-dependent, in terms of gene expression level. This was revealed by inspection of microarray data deposited into the Gene Expression Omnibus (GEO), and confirmed for 7 of the transcripts to date using quantitative PCR analyses (data not shown). Support Vector Machine classification and validation program was applied to the set of 20 novel differentially expressed transcripts that overlapped among all 3 ASD subgroups whose LCL were profiled by DNA microarray analyses. This analysis demonstrated that based upon these 20 novel transcripts alone, samples from the combined autistic groups can be separated from nonautistic control samples with an accuracy of 89.2% (based upon these 20 novel transcripts, the accuracy of class assignment was 89.2% (99/111 correctly assigned)). Therefore, this set of 20 noncoding transcripts will be useful as diagnostic biomarkers of autism, regardless of phenotype.

TABLE 28
Differentially expressed transcripts across all 3 ASD subgroups analyzed.

| | | Map | | | | | |
|---|---|---|---|---|---|---|---|
| GenBank# | Associated gene | Region | log2(L/C) | log2(M/C) | log2(S/C) | log2(A/C) | Adj p value* |
| T65857 | Unknown | intergenic | −0.878 | −0.510 | −0.745 | −0.825 | 2.21E−04 |
| N47010 | KIAA1432 | intronic | −0.802 | −0.486 | −0.747 | −0.763 | 4.52E−03 |
| AI076295 | MEMO1 | intronic | −0.547 | −0.555 | −0.634 | −0.627 | 1.54E−03 |
| H97875 | DENND5B | intronic | −0.361 | −0.488 | −0.465 | −0.477 | 9.43E−04 |
| AA704941 | LARP5 | intergenic | −0.507 | −0.304 | −0.411 | −0.470 | 2.60E−04 |
| AA907052 | SMA4, GUSBP1 | intronic | −0.307 | −0.462 | −0.496 | −0.438 | 3.04E−04 |
| H56961 | JMJD2C | intronic | −0.456 | −0.289 | −0.430 | −0.436 | 2.30E−04 |
| AA995108 | CUL3 | intronic | −0.373 | −0.386 | −0.351 | −0.422 | 3.67E−04 |
| H73587 | XTP2, BAT2D1 | intronic | −0.383 | −0.336 | −0.329 | −0.390 | 2.16E−03 |
| H63175 | USP47 | intronic | −0.232 | −0.302 | −0.370 | −0.357 | 6.94E−04 |
| H25019 | ZZZ3 | intronic | −0.239 | −0.285 | −0.444 | −0.349 | 4.04E−03 |
| AA406078 | ZEB1 | intronic | −0.268 | −0.279 | −0.363 | −0.342 | 9.01E−03 |
| AA906454 | MUDENG | intronic | −0.346 | −0.230 | −0.304 | −0.340 | 2.42E−04 |
| AA026388 | SENP6 | intronic | −0.224 | −0.230 | −0.460 | −0.320 | 1.46E−04 |
| N73227 | PARG | intronic | −0.256 | −0.249 | −0.332 | −0.317 | 5.24E−03 |
| AI276056 | ATP13A3 | intronic | −0.206 | −0.261 | −0.280 | −0.284 | 2.08E−03 |
| R11217 | FBXW7 | intronic | −0.218 | −0.250 | −0.272 | −0.262 | 4.89E−04 |
| N26823 | RBBP6 | | −0.259 | −0.185 | −0.206 | −0.249 | 2.27E−05 |
| AA700707 | ATP11B | intergenic | −0.206 | −0.216 | −0.272 | −0.245 | 3.37E−03 |
| H85885 | KIAA0999 | intronic | −0.171 | −0.225 | −0.238 | −0.232 | 5.47E−03 |

L: severely language impaired;
M: mildly affected;
S: with notable savant skills;
A: all autistic groups combined;
C: nonautistic control group.
*Statistical significance of unpaired t-test comparing controls vs. all autistic probands (A).
The adjusted p-value was obtained using a standard Bonferroni correction for multiple testing.

What is claimed is:

1. A gene chip array having a plurality of different oligonucleotides with specificity for genes associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof, wherein the oligonucleotides are specific for the genes set out in Table 3, Table 7, Table 8, Table 9, Table 10, Table 18, Table 19, Table 21, Table 22, Table 23, Table 25, Table 26, Table 27, or Table 28, or a combination thereof.

2. A method of screening a subject for a neurological disease or disorder comprising the steps of: (a) isolating a nucleic acid, protein or cellular extract from at least one cell from the subject; (b) measuring the gene expression level of at least five different genes in Table 3, Table 7, Table 8, Table 9, Table 10, Table 18, Table 19, Table 21, Table 22, Table 23, Table 25, Table 26, Table 27, or Table 28, or a combination thereof in the sample, wherein the at least five different genes have been determined to have differential expression in subjects with a neurological disease or disorder, wherein the subject is diagnosed to be at risk for or affected by a neurological disease or disorder if there is a statistically significant difference in the gene expression level in the at least five different genes in the sample compared to the gene expression level of the same genes from a healthy individual.

3. The method of claim 2, wherein the neurological disease comprises at least one autism spectrum disorder, autistic disorder, pervasive developmental disorder-not otherwise specified (PDD-NOS) including atypical autism, Asperger's Disorder, or a combination thereof, and wherein the at least 5 different genes in Table 3, Table 7, Table 8, Table 9, Table 10, Table 18, Table 19, Table 21, Table 22, Table 23, Table 25, Table 26, Table 27, or Table 28, or a combination thereof comprise genes involved in nervous system development, axon guidance, synaptic transmission or plasticity, myelination, long-term potentiation, neuron toxicity, embryonic development, regulation of actin networks, digestion, inflammation, oxidative stress, epilepsy, apoptosis, cell survival, differentiation, the unfolded protein response, Type II diabetes and insulin signaling, digestion, liver toxicity (hepatic stellate cell activation, fibrosis, and cholestasis), endocrine function, circadian rhythm, cholesterol metabolism and the steroidogenesis pathway, or a combination thereof.

4. The method of claim 2, wherein the healthy individual is a non-phenotypic discordant twin, sibling of the subject, or unrelated subject.

5. The method of claim 2, wherein the method distinguishes between different variants of autism spectrum disorder comprising a lower severity scores across all ADIR items, an intermediate severity across all ADIR items, a higher severity scores on spoken language items on the ADIR, a higher frequency of savant skills, and a severe language impairment, or a combination thereof.

6. The method of claim 2, wherein the gene expression is quantified with an assay comprising large scale microarray analysis, RT qPCR analysis, quantitative nuclease protection assay (qNPA) analysis, Western analysis, and focused gene chip analysis, in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift assay (EMSA), radioimmunoassay (RIA), fluorescent or histochemical staining, microscopy and digital image analysis, and fluorescence activated cell analysis or sorting (FACS), nucleic acid hybridization, antibody binding, or a combination thereof.

7. A method for determining a gene profile for at least one autism spectrum disorder, comprising (a) preparing samples of control and experimental cDNA, wherein the experimental cDNA is generated from a nucleic acid sample isolated from a subject suspected of being afflicted with the at least one autism spectrum disorder and the control cDNA is generated from a nucleic acid sample isolated from a healthy individual; (b) preparing one or more microarrays comprising a plurality of different oligonucleotides having specificity for genes associated with the at least one autism spectrum disorder; (c) applying the prepared samples to the one or more microarrays to allow hybridization between the oligonucleotides and the control cDNA and the oligonucleotide and the experimental cDNAs; (d) identifying the oligonucleotides on the microarray which display differential hybridization to the experimental cDNA relative to the control cDNA thereby determining a gene profile for the at least one autism spectrum disorder.

8. The method according to claim 7, wherein the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof

9. The method of claim 7, wherein the at least one autism spectrum disorder comprises autistic disorder, pervasive developmental disorder-not otherwise specified (PDD-NOS), including atypical autism, Asperger's Disorder, or a combination thereof.

10. A method for distinguishing between different phenotypes of an autism spectrum disorder comprising severely language impaired (L), mildly affected (M), or “savants” (S) comprising (a) preparing samples of control and experimental cDNA, wherein the experimental cDNA is generated from a nucleic acid sample isolated from a subject suspected of being afflicted with at least one phenotype comprising the severely language impaired (L), mildly affected (M), or “savants” (S); (b) preparing one or more microarrays comprising a plurality of different oligonucleotides having specificity for genes associated with the at least one phenotype; (c) applying the prepared samples to the one or more microarrays to allow hybridization between the oligonucleotides and the control and experimental cDNAs; (d) identifying the oligonucleotides on the microarray which display differential hybridization to the experimental cDNA relative to the control cDNA thereby determining a gene profile for distinguishing among the different phenotypes of autism spectrum disorder.

11. The method according to claim 10, wherein the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

12. The method of claim 10, wherein the at least one autism spectrum disorder comprises autistic disorder, pervasive developmental disorder-not otherwise specified (PDD-NOS), including atypical autism, Asperger's Disorder, or a combination thereof.

13. A method of assessing the efficacy of a treatment in an individual having at least one autism spectrum disorder comprising (a) determining differential gene expression profile data specific for at least five difference genes set out in Table 3, Table 7, Table 8, Table 9, Table 10, Table 18, Table 19, Table 21, Table 22, Table 23, Table 25, Table 26, Table 27, or Table 28, or a combination thereof, in a plurality of patient samples of a selected tissue type; (b) determining a degree of similarity between (a) the differential gene expression profile data in the patient samples; and (b) a differential gene profile specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, produced by a therapy which has been shown to be efficacious in treatment of the at least one autism spectrum disorder; wherein a high degree of similarity of the differential gene expression profile data is indicative that the treatment is effective.

14. A method of determining a gene profile indicative of administration of a therapeutic treatment to a subject with at least one autism spectrum disorder comprising (a) preparing samples of control and experimental cDNA, wherein the experimental cDNA is generated from a nucleic acid sample isolated from a subject who has received the therapeutic treatment; (b) preparing one or more microarrays comprising a plurality of different oligonucleotides, wherein the oligonucleotides are specific to genes associated with an autism spectrum disorder; (c) applying the prepared samples to the one or more microarrays to allow hybridization between the oligonucleotides and the control and experimental cDNAs; (d) identifying the oligonucleotides on the microarray which display differential hybridization to the experimental cDNA relative to the control cDNA thereby determining a gene profile indicative for the administration of the therapeutic treatment to the subject with at least one autism spectrum disorder.

15. The method according to claim 14, wherein the plurality of different oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

16. The method according to claim 14, wherein the at least one autism spectrum disorder neurological condition comprises autistic disorder, pervasive developmental disorder-not otherwise specified (PDD-NOS), including atypical autism, Asperger's Disorder, or a combination thereof.

17. A method for predicting efficacy of a test compound for altering a behavioral response in a subject with at least one autism spectrum disorder comprising: (a) preparing a microarray comprising a plurality of different oligonucleotides, wherein the oligonucleotides are specific to genes associated with an autism spectrum disorder; (b) obtaining a gene profile representative of the gene expression profile of at least one sample of a selected tissue type from a subject subjected to each of at least one of a plurality of selected behavioral therapies which promote the behavioral response; (c) administering the test compound to the subject; and (d) comparing gene expression profile data in at least one sample of the selected tissue type from the subject treated with the test compound to determine a degree of similarity with one or more gene profiles associated with an autism spectrum disorder; wherein the predicted efficacy of the test compound for altering the behavioral response is correlated to said degree of similarity.

18. The method according to claim 17, wherein the plurality of oligonucleotides is specific for at least five different genes set out in Table 3, Table 7, Table 8, Table 9, Table 10, Table 18, Table 19, Table 21, Table 22, Table 23, Table 25, Table 26, Table 27, or Table 28, or a combination thereof.

19. The method according to claim 17, wherein the autism spectrum disorder neurological condition comprises autistic disorder, pervasive developmental disorder-not otherwise specified (PDD-NOS), including atypical autism, Asperger's Disorder, or a combination thereof, and wherein at least one of the selected tissue type of step (b) comprises a neuronal tissue type selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

20. A kit for identifying a compound for treating at least one autism spectrum disorder comprising (a) a database having information stored therein one or more differential gene expression profiles specific for the genes set out in listed in Table 3, Table 7, Table 8, Table 9, Table 10, Table 18, Table 19, Table 21, Table 22, Table 23, Table 25, Table 26, Table 27, or Table 28, or a combination thereof, of subjects that have been subjected to at least one of a plurality of selected autism spectrum disorder neurological therapies and wherein the subject has undergone a desired physiological change; and (b) a computer program for comparing gene expression profile data obtained from assays wherein a test compound is administered to a subject with the database and providing information representative of a measure of similarity between the gene expression profile data and one or more stored gene profiles.
